SEQUENCE LISTING (297 sequences)

SEQ ID NO: 1 - protein, 158 aa, Human papillomavirus type 16
  E6 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2 - protein, 98 aa, Human papillomavirus type 16
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 158 aa, Human papillomavirus type 18
  E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 4 - protein, 105 aa, Human papillomavirus type 18
  E7 protein
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 5 - protein, 30 aa, Human papillomavirus type 16
  E6 protein aa 1-30
MHQKRTAMFQDPQERPRKLPQLCTELQTTI

SEQ ID NO: 6 - protein, 9 aa, Human papillomavirus type 16
  E6 protein aa 7-15
AMFQDPQER

SEQ ID NO: 7 - protein, 11 aa, Human papillomavirus type 16
  E6 protein aa 8-18
MFQDPQERPRK

SEQ ID NO: 8 - protein, 10 aa, Human papillomavirus type 16
  E6 protein aa 9-18
FQDPQERPRK

SEQ ID NO: 9 - protein, 9 aa, Human papillomavirus type 16
  E6 protein aa 11-19
DPQERPRKL

SEQ ID NO: 10 - protein, 22 aa, Human papillomavirus type 16
  E6 protein aa 11-32
DPQERPRKLPQLCTELQTTIHD

SEQ ID NO: 11 - protein, 10 aa, Human papillomavirus type 16
  E6 protein aa 13-22
QERPRKLPQL

SEQ ID NO: 12 - protein, 8 aa, Human papillomavirus type 16
  E6 protein aa 15-22
RPRKLPQL

SEQ ID NO: 13 - protein, 9 aa, Human papillomavirus type 16
  E6 protein aa 15-23
RPRKLPQLC

SEQ ID NO: 14 - protein, 10 aa, Human papillomavirus type 16
  E6 protein aa 15-24
RPRKLPQLCT

SEQ ID NO: 15 - protein, 11 aa, Human papillomavirus type 16
  E6 protein aa 15-25
RPRKLPQLCTE

SEQ ID NO: 16 - protein, 9 aa, Human papillomavirus type 16
  E6 protein aa 18-26
KLPQLCTEL

SEQ ID NO: 17 - protein, 11 aa, Human papillomavirus type 16
  E6 protein aa 18-28
KLPQLCTELQT

SEQ ID NO: 18 - protein, 8 aa, Human papillomavirus type 16
  E6 protein aa 19-26
LPQLCTEL

SEQ ID NO: 19 - protein, 10 aa, Human papillomavirus type 16
  E6 protein aa 19-28
LPQLCTELQT

SEQ ID NO: 20 - protein, 9 aa, Human papillomavirus type 16
  E6 protein aa 23-31
CTELQTTIH

SEQ ID NO: 21 - protein, 9 aa, Human papillomavirus type 16
  E6 protein aa 25-33
ELQTTIHDI

SEQ ID NO: 22 - protein, 9 aa, Human papillomavirus type 16
  E6 protein aa 26-34
LQTTIHDII

SEQ ID NO: 23 - protein, 11 aa, Human papillomavirus type 16
  E6 protein aa 28-38
TTIHDIILECV

SEQ ID NO: 24 - protein, 9 aa, Human papillomavirus type 16
  E6 protein aa 29-37
TIHDIILEC

SEQ ID NO: 25 - protein, 10 aa, Human papillomavirus type 16
  E6 protein aa 29-38
TIHDIILECV

SEQ ID NO: 26 - protein, 11 aa, Human papillomavirus type 16
  E6 protein aa 29-39
TIHDIILECVY

SEQ ID NO: 27 - protein, 10 aa, Human papillomavirus type 16
  E6 protein aa 30-39
IHDIILECVY

SEQ ID NO: 28 - protein, 8 aa, Human papillomavirus type 16
  E6 protein aa 31-38
HDIILECV

SEQ ID NO: 29 - protein, 9 aa, Human papillomavirus type 16
  E6 protein aa 31-39
HDIILECVY

SEQ ID NO: 30 - protein, 11 aa, Human papillomavirus type 16
  E6 protein aa 31-41
HDIILECVYCK

SEQ ID NO: 31 - protein, 10 aa, Human papillomavirus type 16
  E6 protein aa 32-41
DIILECVYCK

SEQ ID NO: 32 - protein, 9 aa, Human papillomavirus type 16
  E6 protein aa 33-41
IILECVYCK

SEQ ID NO: 33 - protein, 8 aa, Human papillomavirus type 16
  E6 protein aa 34-41
ILECVYCK

SEQ ID NO: 34 - protein, 11 aa, Human papillomavirus type 16
  E6 protein aa 34-44
ILECVYCKQQL

SEQ ID NO: 35 - protein, 10 aa, Human papillomavirus type 16
  E6 protein aa 35-44
LECVYCKQQL

SEQ ID NO: 36 - protein, 10 aa, Human papillomavirus type 16
  E6 protein aa 37-46
CVYCKQQLLR

SEQ ID NO: 37 - protein, 18 aa, Human papillomavirus type 16
  E6 protein aa 37-54
CVYCKQQLLRREVYDFAF

SEQ ID NO: 38 - protein, 32 aa, Human papillomavirus type 16
  E6 protein aa 37-68
CVYCKQQLLRREVYDFAFRDLCIVYRDGNPYA

SEQ ID NO: 39 - protein, 8 aa, Human papillomavirus type 16
  E6 protein aa 38-45
VYCKQQLL

SEQ ID NO: 40 - protein, 9 aa, Human papillomavirus type 16
  E6 protein aa 38-46
VYCKQQLLR

SEQ ID NO: 41 - protein, 10 aa, Human papillomavirus type 16
  E6 protein aa 38-47
VYCKQQLLRR

SEQ ID NO: 42 - protein, 11 aa, Human papillomavirus type 16
  E6 protein aa 40-50
CKQQLLRREVY

SEQ ID NO: 43 - protein, 10 aa, Human papillomavirus type 16
  E6 protein aa 41-50
KQQLLRREVY

SEQ ID NO: 44 - protein, 9 aa, Human papillomavirus type 16
  E6 protein aa 42-50
QQLLRREVY

SEQ ID NO: 45 - protein, 11 aa, Human papillomavirus type 16
  E6 protein aa 42-52
QQLLRREVYDF

SEQ ID NO: 46 - protein, 10 aa, Human papillomavirus type 16
  E6 protein aa 43-52
QLLRREVYDF

SEQ ID NO: 47 - protein, 15 aa, Human papillomavirus type 16
  E6 protein aa 43-57
QLLRREVYDFAFRDL

SEQ ID NO: 48 - protein, 9 aa, Human papillomavirus type 16
  E6 protein aa 44-52
LLRREVYDF

SEQ ID NO: 49 - protein, 11 aa, Human papillomavirus type 16
  E6 protein aa 44-54
LLRREVYDFAF

SEQ ID NO: 50 - protein, 24 aa, Human papillomavirus type 16
  E6 protein aa 45-68
LRREVYDFAFRDLCIVYRDGNPYA

SEQ ID NO: 51 - protein, 8 aa, Human papillomavirus type 16
  E6 protein aa 47-54
REVYDFAF

SEQ ID NO: 52 - protein, 8 aa, Human papillomavirus type 16
  E6 protein aa 48-55
EVYDFAFR

SEQ ID NO: 53 - protein, 10 aa, Human papillomavirus type 16
  E6 protein aa 48-57
EVYDFAFRDL

SEQ ID NO: 54 - protein, 9 aa, Human papillomavirus type 16
  E6 protein aa 49-57
VYDFAFRDL

SEQ ID NO: 55 - protein, 10 aa, Human papillomavirus type 16
  E6 protein aa 49-58
VYDFAFRDLC

SEQ ID NO: 56 - protein, 11 aa, Human papillomavirus type 16
  E6 protein aa 49-59
VYDFAFRDLCI

SEQ ID NO: 57 - protein, 10 aa, Human papillomavirus type 16
  E6 protein aa 50-59
YDFAFRDLCI

SEQ ID NO: 58 - protein, 9 aa, Human papillomavirus type 16
  E6 protein aa 51-59
DFAFRDLCI

SEQ ID NO: 59 - protein, 9 aa, Human papillomavirus type 16
  E6 protein aa 52-60
FAFRDLCIV

SEQ ID NO: 60 - protein, 10 aa, Human papillomavirus type 16
  E6 protein aa 52-61
FAFRDLCIVY

SEQ ID NO: 61 - protein, 11 aa, Human papillomavirus type 16
  E6 protein aa 52-62
FAFRDLCIVYR

SEQ ID NO: 62 - protein, 9 aa, Human papillomavirus type 16
  E6 protein aa 53-61
AFRDLCIVY

SEQ ID NO: 63 - protein, 10 aa, Human papillomavirus type 16
  E6 protein aa 53-62
AFRDLCIVYR

SEQ ID NO: 64 - protein, 15 aa, Human papillomavirus type 16
  E6 protein aa 54-68
FRDLCIVYRDGNPYA

SEQ ID NO: 65 - protein, 32 aa, Human papillomavirus type 16
  E6 protein aa 55-86
RDLCIVYRDGNPYAVCDKCLKFYSKISEYRHY

SEQ ID NO: 66 - protein, 11 aa, Human papillomavirus type 16
  E6 protein aa 57-67
LCIVYRDGNPY

SEQ ID NO: 67 - protein, 10 aa, Human papillomavirus type 16
  E6 protein aa 58-67
CIVYRDGNPY

SEQ ID NO: 68 - protein, 9 aa, Human papillomavirus type 16
  E6 protein aa 59-67
IVYRDGNPY

SEQ ID NO: 69 - protein, 10 aa, Human papillomavirus type 16
  E6 protein aa 59-68
IVYRDGNPYA

SEQ ID NO: 70 - protein, 11 aa, Human papillomavirus type 16
  E6 protein aa 59-69
IVYRDGNPYAV

SEQ ID NO: 71 - protein, 8 aa, Human papillomavirus type 16
  E6 protein aa 60-67
VYRDGNPY

SEQ ID NO: 72 - protein, 10 aa, Human papillomavirus type 16
  E6 protein aa 60-69
VYRDGNPYAV

SEQ ID NO: 73 - protein, 22 aa, Human papillomavirus type 16
  E6 protein aa 61-82
YRDGNPYAVCDKCLKFYSKISE

SEQ ID NO: 74 - protein, 11 aa, Human papillomavirus type 16
  E6 protein aa 65-75
NPYAVCDKCLK

SEQ ID NO: 75 - protein, 9 aa, Human papillomavirus type 16
  E6 protein aa 66-74
PYAVCDKCL

SEQ ID NO: 76 - protein, 10 aa, Human papillomavirus type 16
  E6 protein aa 66-75
PYAVCDKCLK

SEQ ID NO: 77 - protein, 11 aa, Human papillomavirus type 16
  E6 protein aa 66-76
PYAVCDKCLKF

SEQ ID NO: 78 - protein, 9 aa, Human papillomavirus type 16
  E6 protein aa 67-75
YAVCDKCLK

SEQ ID NO: 79 - protein, 10 aa, Human papillomavirus type 16
  E6 protein aa 67-76
YAVCDKCLKF

SEQ ID NO: 80 - protein, 11 aa, Human papillomavirus type 16
  E6 protein aa 67-77
YAVCDKCLKFY

SEQ ID NO: 81 - protein, 8 aa, Human papillomavirus type 16
  E6 protein aa 68-75
AVCDKCLK

SEQ ID NO: 82 - protein, 9 aa, Human papillomavirus type 16
  E6 protein aa 68-76
AVCDKCLKF

SEQ ID NO: 83 - protein, 10 aa, Human papillomavirus type 16
  E6 protein aa 68-77
AVCDKCLKFY

SEQ ID NO: 84 - protein, 11 aa, Human papillomavirus type 16
  E6 protein aa 68-78
AVCDKCLKFYS

SEQ ID NO: 85 - protein, 11 aa, Human papillomavirus type 16
  E6 protein aa 69-79
VCDKCLKFYSK

SEQ ID NO: 86 - protein, 8 aa, Human papillomavirus type 16
  E6 protein aa 71-78
DKCLKFYS

SEQ ID NO: 87 - protein, 9 aa, Human papillomavirus type 16
  E6 protein aa 72-80
KCLKFYSKI

SEQ ID NO: 88 - protein, 11 aa, Human papillomavirus type 16
  E6 protein aa 73-83
CLKFYSKISEY

SEQ ID NO: 89 - protein, 33 aa, Human papillomavirus type 16
  E6 protein aa 73-105
CLKFYSKISEYRHYCYSLYGTTLEQQYNKPLCD

SEQ ID NO: 90 - protein, 10 aa, Human papillomavirus type 16
  E6 protein aa 74-83
LKFYSKISEY

SEQ ID NO: 91 - protein, 15 aa, Human papillomavirus type 16
  E6 protein aa 74-88
LKFYSKISEYRHYCY

SEQ ID NO: 92 - protein, 9 aa, Human papillomavirus type 16
  E6 protein aa 75-83
KFYSKISEY

SEQ ID NO: 93 - protein, 10 aa, Human papillomavirus type 16
  E6 protein aa 75-84
KFYSKISEYR

SEQ ID NO: 94 - protein, 11 aa, Human papillomavirus type 16
  E6 protein aa 75-85
KFYSKISEYRH

SEQ ID NO: 95 - protein, 8 aa, Human papillomavirus type 16
  E6 protein aa 76-83
FYSKISEY

SEQ ID NO: 96 - protein, 10 aa, Human papillomavirus type 16
  E6 protein aa 76-85
FYSKISEYRH

SEQ ID NO: 97 - protein, 11 aa, Human papillomavirus type 16
  E6 protein aa 76-86
FYSKISEYRHY

SEQ ID NO: 98 - protein, 10 aa, Human papillomavirus type 16
  E6 protein aa 77-86
YSKISEYRHY

SEQ ID NO: 99 - protein, 9 aa, Human papillomavirus type 16
  E6 protein aa 78-86
SKISEYRHY

SEQ ID NO: 100 - protein, 11 aa, Human papillomavirus type 16
  E6 protein aa 78-88
SKISEYRHYCY

SEQ ID NO: 101 - protein, 8 aa, Human papillomavirus type 16
  E6 protein aa 79-86
KISEYRHY

SEQ ID NO: 102 - protein, 9 aa, Human papillomavirus type 16
  E6 protein aa 79-87
KISEYRHYC

SEQ ID NO: 103 - protein, 10 aa, Human papillomavirus type 16
  E6 protein aa 79-88
KISEYRHYCY

SEQ ID NO: 104 - protein, 9 aa, Human papillomavirus type 16
  E6 protein aa 80-88
ISEYRHYCY

SEQ ID NO: 105 - protein, 8 aa, Human papillomavirus type 16
  E6 protein aa 81-88
SEYRHYCY

SEQ ID NO: 106 - protein, 10 aa, Human papillomavirus type 16
  E6 protein aa 81-90
SEYRHYCYSL

SEQ ID NO: 107 - protein, 11 aa, Human papillomavirus type 16
  E6 protein aa 81-91
SEYRHYCYSLY

SEQ ID NO: 108 - protein, 9 aa, Human papillomavirus type 16
  E6 protein aa 82-90
EYRHYCYSL

SEQ ID NO: 109 - protein, 10 aa, Human papillomavirus type 16
  E6 protein aa 82-91
EYRHYCYSLY

SEQ ID NO: 110 - protein, 8 aa, Human papillomavirus type 16
  E6 protein aa 83-90
YRHYCYSL

SEQ ID NO: 111 - protein, 9 aa, Human papillomavirus type 16
  E6 protein aa 83-91
YRHYCYSLY

SEQ ID NO: 112 - protein, 8 aa, Human papillomavirus type 16
  E6 protein aa 84-91
RHYCYSLY

SEQ ID NO: 113 - protein, 11 aa, Human papillomavirus type 16
  E6 protein aa 84-94
RHYCYSLYGTT

SEQ ID NO: 114 - protein, 11 aa, Human papillomavirus type 16
  E6 protein aa 85-95
HYCYSLYGTTL

SEQ ID NO: 115 - protein, 11 aa, Human papillomavirus type 16
  E6 protein aa 86-96
YCYSLYGTTLE

SEQ ID NO: 116 - protein, 9 aa, Human papillomavirus type 16
  E6 protein aa 87-95
CYSLYGTTL

SEQ ID NO: 117 - protein, 10 aa, Human papillomavirus type 16
  E6 protein aa 87-96
CYSLYGTTLE

SEQ ID NO: 118 - protein, 8 aa, Human papillomavirus type 16
  E6 protein aa 88-95
YSLYGTTL

SEQ ID NO: 119 - protein, 11 aa, Human papillomavirus type 16
  E6 protein aa 89-99
SLYGTTLEQQY

SEQ ID NO: 120 - protein, 10 aa, Human papillomavirus type 16
  E6 protein aa 90-99
LYGTTLEQQY

SEQ ID NO: 121 - protein, 10 aa, Human papillomavirus type 16
  E6 protein aa 91-100
YGTTLEQQYN

SEQ ID NO: 122 - protein, 11 aa, Human papillomavirus type 16
  E6 protein aa 91-101
YGTTLEQQYNK

SEQ ID NO: 123 - protein, 22 aa, Human papillomavirus type 16
  E6 protein aa 91-112
YGTTLEQQYNKPLCDLLIRCIN

SEQ ID NO: 124 - protein, 8 aa, Human papillomavirus type 16
  E6 protein aa 92-99
GTTLEQQY

SEQ ID NO: 125 - protein, 10 aa, Human papillomavirus type 16
  E6 protein aa 92-101
GTTLEQQYNK

SEQ ID NO: 126 - protein, 9 aa, Human papillomavirus type 16
  E6 protein aa 93-101
TTLEQQYNK

SEQ ID NO: 127 - protein, 8 aa, Human papillomavirus type 16
  E6 protein aa 94-101
TLEQQYNK

SEQ ID NO: 128 - protein, 9 aa, Human papillomavirus type 16
  E6 protein aa 95-103
LEQQYNKPL

SEQ ID NO: 129 - protein, 10 aa, Human papillomavirus type 16
  E6 protein aa 97-106
QQYNKPLCDL

SEQ ID NO: 130 - protein, 9 aa, Human papillomavirus type 16
  E6 protein aa 98-106
QYNKPLCDL

SEQ ID NO: 131 - protein, 10 aa, Human papillomavirus type 16
  E6 protein aa 98-107
QYNKPLCDLL

SEQ ID NO: 132 - protein, 11 aa, Human papillomavirus type 16
  E6 protein aa 98-108
QYNKPLCDLLI

SEQ ID NO: 133 - protein, 8 aa, Human papillomavirus type 16
  E6 protein aa 99-106
YNKPLCDL

SEQ ID NO: 134 - protein, 8 aa, Human papillomavirus type 16
  E6 protein aa 101-108
KPLCDLLI

SEQ ID NO: 135 - protein, 11 aa, Human papillomavirus type 16
  E6 protein aa 101-111
KPLCDLLIRCI

SEQ ID NO: 136 - protein, 22 aa, Human papillomavirus type 16
  E6 protein aa 101-122
KPLCDLLIRCINCQKPLCPEEK

SEQ ID NO: 137 - protein, 11 aa, Human papillomavirus type 16
  E6 protein aa 105-115
DLLIRCINCQK

SEQ ID NO: 138 - protein, 10 aa, Human papillomavirus type 16
  E6 protein aa 106-115
LLIRCINCQK

SEQ ID NO: 139 - protein, 9 aa, Human papillomavirus type 16
  E6 protein aa 107-115
LIRCINCQK

SEQ ID NO: 140 - protein, 11 aa, Human papillomavirus type 16
  E6 protein aa 107-117
LIRCINCQKPL

SEQ ID NO: 141 - protein, 11 aa, Human papillomavirus type 16
  E6 protein aa 109-119
RCINCQKPLCP

SEQ ID NO: 142 - protein, 9 aa, Human papillomavirus type 16
  E6 protein aa 113-121
CQKPLCPEE

SEQ ID NO: 143 - protein, 9 aa, Human papillomavirus type 16
  E6 protein aa 118-126
CPEEKQRHL

SEQ ID NO: 144 - protein, 22 aa, Human papillomavirus type 16
  E6 protein aa 121-142
EKQRHLDKKQRFHNIRGRWTGR

SEQ ID NO: 145 - protein, 11 aa, Human papillomavirus type 16
  E6 protein aa 122-132
KQRHLDKKQRF

SEQ ID NO: 146 - protein, 9 aa, Human papillomavirus type 16
  E6 protein aa 125-133
HLDKKQRFH

SEQ ID NO: 147 - protein, 11 aa, Human papillomavirus type 16
  E6 protein aa 125-135
HLDKKQRFHNI

SEQ ID NO: 148 - protein, 8 aa, Human papillomavirus type 16
  E6 protein aa 127-134
DKKQRFHN

SEQ ID NO: 149 - protein, 9 aa, Human papillomavirus type 16
  E6 protein aa 127-135
DKKQRFHNI

SEQ ID NO: 150 - protein, 15 aa, Human papillomavirus type 16
  E6 protein aa 127-141
DKKQRFHNIRGRWTG

SEQ ID NO: 151 - protein, 8 aa, Human papillomavirus type 16
  E6 protein aa 128-135
KKQRFHNI

SEQ ID NO: 152 - protein, 9 aa, Human papillomavirus type 16
  E6 protein aa 128-136
KKQRFHNIR

SEQ ID NO: 153 - protein, 10 aa, Human papillomavirus type 16
  E6 protein aa 129-138
KQRFHNIRGR

SEQ ID NO: 154 - protein, 11 aa, Human papillomavirus type 16
  E6 protein aa 129-139
KQRFHNIRGRW

SEQ ID NO: 155 - protein, 9 aa, Human papillomavirus type 16
  E6 protein aa 131-139
RFHNIRGRW

SEQ ID NO: 156 - protein, 11 aa, Human papillomavirus type 16
  E6 protein aa 134-144
NIRGRWTGRCM

SEQ ID NO: 157 - protein, 9 aa, Human papillomavirus type 16
  E6 protein aa 136-144
RGRWTGRCM

SEQ ID NO: 158 - protein, 10 aa, Human papillomavirus type 16
  E6 protein aa 137-146
GRWTGRCMSC

SEQ ID NO: 159 - protein, 10 aa, Human papillomavirus type 16
  E6 protein aa 139-148
WTGRCMSCCR

SEQ ID NO: 160 - protein, 10 aa, Human papillomavirus type 16
  E6 protein aa 142-151
RCMSCCRSSR

SEQ ID NO: 161 - protein, 11 aa, Human papillomavirus type 16
  E6 protein aa 148-158
RSSRTRRETQL

SEQ ID NO: 162 - protein, 10 aa, Human papillomavirus type 16
  E6 protein aa 149-158
SSRTRRETQL

SEQ ID NO: 163 - protein, 8 aa, Human papillomavirus type 16
  E6 protein aa 151-158
RTRRETQL

SEQ ID NO: 164 - protein, 12 aa, Human papillomavirus type 16
  E7 protein aa 1-12
MHGDTPTLHEYM

SEQ ID NO: 165 - protein, 10 aa, Human papillomavirus type 16
  E7 protein aa 2-11
HGDTPTLHEY

SEQ ID NO: 166 - protein, 8 aa, Human papillomavirus type 16
  E7 protein aa 4-11
DTPTLHEY

SEQ ID NO: 167 - protein, 8 aa, Human papillomavirus type 16
  E7 protein aa 5-12
TPTLHEYM

SEQ ID NO: 168 - protein, 9 aa, Human papillomavirus type 16
  E7 protein aa 5-13
TPTLHEYML

SEQ ID NO: 169 - protein, 14 aa, Human papillomavirus type 16
  E7 protein aa 5-18
TPTLHEYMLDLQPE

SEQ ID NO: 170 - protein, 9 aa, Human papillomavirus type 16
  E7 protein aa 7-15
TLHEYMLDL

SEQ ID NO: 171 - protein, 11 aa, Human papillomavirus type 16
  E7 protein aa 7-17
TLHEYMLDLQP

SEQ ID NO: 172 - protein, 21 aa, Human papillomavirus type 16
  E7 protein aa 7-27
TLHEYMLDLQPETTDLYCYEQ

SEQ ID NO: 173 - protein, 8 aa, Human papillomavirus type 16
  E7 protein aa 11-18
YMLDLQPE

SEQ ID NO: 174 - protein, 9 aa, Human papillomavirus type 16
  E7 protein aa 11-19
YMLDLQPET

SEQ ID NO: 175 - protein, 10 aa, Human papillomavirus type 16
  E7 protein aa 11-20
YMLDLQPETT

SEQ ID NO: 176 - protein, 11 aa, Human papillomavirus type 16
  E7 protein aa 11-21
YMLDLQPETTD

SEQ ID NO: 177 - protein, 16 aa, Human papillomavirus type 16
  E7 protein aa 11-26
YMLDLQPETTDLYCYE

SEQ ID NO: 178 - protein, 8 aa, Human papillomavirus type 16
  E7 protein aa 12-19
MLDLQPET

SEQ ID NO: 179 - protein, 8 aa, Human papillomavirus type 16
  E7 protein aa 12-20
MLDLQPET

SEQ ID NO: 180 - protein, 15 aa, Human papillomavirus type 16
  E7 protein aa 12-26
MLDLQPETTDLYCYE

SEQ ID NO: 181 - protein, 10 aa, Human papillomavirus type 16
  E7 protein aa 14-23
DLQPETTDLY

SEQ ID NO: 182 - protein, 9 aa, Human papillomavirus type 16
  E7 protein aa 15-23
LQPETTDLY

SEQ ID NO: 183 - protein, 10 aa, Human papillomavirus type 16
  E7 protein aa 15-24
LQPETTDLYC

SEQ ID NO: 184 - protein, 11 aa, Human papillomavirus type 16
  E7 protein aa 15-25
LQPETTDLYCY

SEQ ID NO: 185 - protein, 22 aa, Human papillomavirus type 16
  E7 protein aa 17-38
PETTDLYCYEQLNDSSEEEDEI

SEQ ID NO: 186 - protein, 8 aa, Human papillomavirus type 16
  E7 protein aa 18-25
ETTDLYCY

SEQ ID NO: 187 - protein, 9 aa, Human papillomavirus type 16
  E7 protein aa 18-26
ETTDLYCYE

SEQ ID NO: 188 - protein, 9 aa, Human papillomavirus type 16
  E7 protein aa 19-27
TTDLYCYEQ

SEQ ID NO: 189 - protein, 11 aa, Human papillomavirus type 16
  E7 protein aa 19-29
TTDLYCYEQLN

SEQ ID NO: 190 - protein, 20 aa, Human papillomavirus type 16
  E7 protein aa 21-40
DLYCYEQLNDSSEEEDEIDG

SEQ ID NO: 191 - protein, 22 aa, Human papillomavirus type 16
  E7 protein aa 21-42
DLYCYEQLNDSSEEEDEIDGPA

SEQ ID NO: 192 - protein, 16 aa, Human papillomavirus type 16
  E7 protein aa 35-50
EDEIDGPAGQAEPDRA

SEQ ID NO: 193 - protein, 11 aa, Human papillomavirus type 16
  E7 protein 42-52
AGQAEPDRAHY

SEQ ID NO: 194 - protein, 9 aa, Human papillomavirus type 16
  E7 protein aa 43-51
GQAEPDRAH

SEQ ID NO: 195 - protein, 10 aa, Human papillomavirus type 16
  E7 protein aa 43-52
GQAEPDRAHY

SEQ ID NO: 196 - protein, 11 aa, Human papillomavirus type 16
  E7 protein aa 43-53
GQAEPDRAHYN

SEQ ID NO: 197 - protein, 35 aa, Human papillomavirus type 16
  E7 protein aa 43-77
GQAEPDRAHYNIVTFCCKCDSTLRLCVQSTHVDIR

SEQ ID NO: 198 - protein, 9 aa, Human papillomavirus type 16
  E7 protein aa 44-52
QAEPDRAHY

SEQ ID NO: 199 - protein, 19 aa, Human papillomavirus type 16
  E7 protein aa 44-62
QAEPDRAHYNIVTFCCKCD

SEQ ID NO: 200 - protein, 10 aa, Human papillomavirus type 16
  E7 protein aa 46-55
EPDRAHYNIV

SEQ ID NO: 201 - protein, 11 aa, Human papillomavirus type 16
  E7 protein aa 47-57
PDRAHYNIVTF

SEQ ID NO: 202 - protein, 10 aa, Human papillomavirus type 16
  E7 protein aa 48-57
DRAHYNIVTF

SEQ ID NO: 203 - protein, 15 aa, Human papillomavirus type 16
  E7 protein aa 48-62
DRAHYNIVTFCCKCD

SEQ ID NO: 204 - protein, 9 aa, Human papillomavirus type 16
  E7 protein aa 49-57
RAHYNIVTF

SEQ ID NO: 205 - protein, 8 aa, Human papillomavirus type 16
  E7 protein aa 50-57
AHYNIVTF

SEQ ID NO: 206 - protein, 11 aa, Human papillomavirus type 16
  E7 protein aa 50-60
AHYNIVTFCCK

SEQ ID NO: 207 - protein, 13 aa, Human papillomavirus type 16
  E7 protein aa 50-62
AHYNIVTFCCKCD

SEQ ID NO: 208 - protein, 8 aa, Human papillomavirus type 16
  E7 protein aa 51-58
HYNIVTFC

SEQ ID NO: 209 - protein, 9 aa, Human papillomavirus type 16
  E7 protein aa 51-59
HYNIVTFCC

SEQ ID NO: 210 - protein, 22 aa, Human papillomavirus type 16
  E7 protein aa 51-72
HYNIVTFCCKCDSTLRLCVQST

SEQ ID NO: 211 - protein, 9 aa, Human papillomavirus type 16
  E7 protein aa 52-60
YNIVTFCCK

SEQ ID NO: 212 - protein, 8 aa, Human papillomavirus type 16
  E7 protein aa 53-60
NIVTFCCK

SEQ ID NO: 213 - protein, 10 aa, Human papillomavirus type 16
  E7 protein aa 56-65
TFCCKCDSTL

SEQ ID NO: 214 - protein, 14 aa, Human papillomavirus type 16
  E7 protein aa 62-75
DSTLRLCVQSTHVD

SEQ ID NO: 215 - protein, 15 aa, Human papillomavirus type 16
  E7 protein aa 64-78
TLRLCVQSTHVDIRT

SEQ ID NO: 216 - protein, 9 aa, Human papillomavirus type 16
  E7 protein aa 66-74
RLCVQSTHV

SEQ ID NO: 217 - protein, 10 aa, Human papillomavirus type 16
  E7 protein aa 67-76
LCVQSTHVDI

SEQ ID NO: 218 - protein, 32 aa, Human papillomavirus type 16
  E7 protein aa 67-98
LCVQSTHVDIRTLEDLLMGTLGIVCPICSQKP

SEQ ID NO: 219 - protein, 8 aa, Human papillomavirus type 16
  E7 protein aa 69-76
VQSTHVDI

SEQ ID NO: 220 - protein, 18 aa, Human papillomavirus type 16
  E7 protein aa 69-86
VQSTHVDIRTLEDLLMGT

SEQ ID NO: 221 - protein, 15 aa, Human papillomavirus type 16
  E7 protein aa 71-85
STHVDIRTLEDLLMG

SEQ ID NO: 222 - protein, 26 aa, Human papillomavirus type 16
  E7 protein aa 72-97
THVDIRTLEDLLMGTLGIVCPICSQK

SEQ ID NO: 223 - protein, 12 aa, Human papillomavirus type 16
  E7 protein aa 73-84
HVDIRTLEDLLM

SEQ ID NO: 224 - protein, 11 aa, Human papillomavirus type 16
  E7 protein aa 76-86
IRTLEDLLMGT

SEQ ID NO: 225 - protein, 10 aa, Human papillomavirus type 16
  E7 protein aa 77-86
RTLEDLLMGT

SEQ ID NO: 226 - protein, 11 aa, Human papillomavirus type 16
  E7 protein aa 77-87
RTLEDLLMGTL

SEQ ID NO: 227 - protein, 9 aa, Human papillomavirus type 16
  E7 protein aa 78-86
TLEDLLMGT

SEQ ID NO: 228 - protein, 9 aa, Human papillomavirus type 16
  E7 protein aa 79-87
LEDLLMGTL

SEQ ID NO: 229 - protein, 11 aa, Human papillomavirus type 16
  E7 protein aa 80-90
EDLLMGTLGIV

SEQ ID NO: 230 - protein, 10 aa, Human papillomavirus type 16
  E7 protein aa 81-90
DLLMGTLGIV

SEQ ID NO: 231 - protein, 11 aa, Human papillomavirus type 16
  E7 protein aa 81-91
DLLMGTLGIVC

SEQ ID NO: 232 - protein, 8 aa, Human papillomavirus type 16
  E7 protein aa 82-89
LLMGTLGI

SEQ ID NO: 233 - protein, 9 aa, Human papillomavirus type 16
  E7 protein aa 82-90
LLMGTLGIV

SEQ ID NO: 234 - protein, 10 aa, Human papillomavirus type 16
  E7 protein aa 82-91
LLMGTLGIVC

SEQ ID NO: 235 - protein, 11 aa, Human papillomavirus type 16
  E7 protein aa 82-92
LLMGTLGIVCP

SEQ ID NO: 236 - protein, 11 aa, Human papillomavirus type 16
  E7 protein aa 83-93
LMGTLGIVCPI

SEQ ID NO: 237 - protein, 10 aa, Human papillomavirus type 16
  E7 protein aa 84-93
MGTLGIVCPI

SEQ ID NO: 238 - protein, 9 aa, Human papillomavirus type 16
  E7 protein aa 85-93
GTLGIVCPI

SEQ ID NO: 239 - protein, 8 aa, Human papillomavirus type 16
  E7 protein aa 86-93
TLGIVCPI

SEQ ID NO: 240 - protein, 9 aa, Human papillomavirus type 16
  E7 protein aa 86-94
TLGIVCPIC

SEQ ID NO: 241 - protein, 11 aa, Human papillomavirus type 16
  E7 protein aa 87-97
LGIVCPICSQK

SEQ ID NO: 242 - protein, 10 aa, Human papillomavirus type 16
  E7 protein aa 88-97
GIVCPICSQK

SEQ ID NO: 243 - protein, 9 aa, Human papillomavirus type 16
  E7 protein aa 89-97
IVCPICSQK

SEQ ID NO: 244 - protein, 9 aa, Human papillomavirus type 16
  E7 protein aa 90-98
VCPICSQKP

SEQ ID NO: 245 - protein, 9 aa, Human papillomavirus type 18
  E6 protein aa 13-21
KLPDLCTEL

SEQ ID NO: 246 - protein, 8 aa, Human papillomavirus type 18
  E6 protein aa 36-43
KTVLELTE

SEQ ID NO: 247 - protein, 9 aa, Human papillomavirus type 18
  E6 protein aa 40-48
ELTEVFEFA

SEQ ID NO: 248 - protein, 15 aa, Human papillomavirus type 18
  E6 protein aa 43-57
EVFEFAFKDLFVVYR

SEQ ID NO: 249 - protein, 22 aa, Human papillomavirus type 18
  E6 protein aa 51-72
DLFVVYRDSIPHAACHKCIDFY

SEQ ID NO: 250 - protein, 22 aa, Human papillomavirus type 18
  E6 protein aa 71-92
FYSRIRELRHYSDSVYGDTLEK

SEQ ID NO: 251 - protein, 11 aa, Human papillomavirus type 18
  E6 protein aa 125-135
RRFHNIAGHYR

SEQ ID NO: 252 - protein, 32 aa, Human papillomavirus type 18
  E7 protein aa 1-32
MHGPKATLQDIVLHLEPQNEIPVDLLCHEQLS

SEQ ID NO: 253 - protein, 12 aa, Human papillomavirus type 18
  E7 protein aa 5-16
KATLQDIVLHLE

SEQ ID NO: 254 - protein, 9 aa, Human papillomavirus type 18
  E7 protein aa 7-15
TLQDIVLHL

SEQ ID NO: 255 - protein, 22 aa, Human papillomavirus type 18
  E7 protein aa 21-42
IPVDLLCHEQLSDSEEENDEID

SEQ ID NO: 256 - protein, 9 aa, Human papillomavirus type 18
  E7 protein aa 86-94
FQQLFLNTL

SEQ ID NO: 257 - protein, 10 aa, Human papillomavirus type 18
  E7 protein aa 88-97
QLFLNTLSFV

SEQ ID NO: 258 - protein, 9 aa, Human papillomavirus type 16
  E6V (R17I) aa 9-17
FQDPQERPI

SEQ ID NO: 259 - protein, 11 aa, Human papillomavirus type 16
  E6V (R17I) aa 9-19
FQDPQERPIKL

SEQ ID NO: 260 - protein, 11 aa, Human papillomavirus type 16
  E6V (R17T) aa 15-25
RPTKLPQLCTE

SEQ ID NO: 261 - protein, 11 aa, Human papillomavirus type 16
  E6V (Q21D) aa 18-28
KLPDLCTELQT

SEQ ID NO: 262 - protein, 9 aa, Human papillomavirus type 16
  E6V (D32E) aa 25-33
ELQTTIHEI

SEQ ID NO: 263 - protein, 9 aa, Human papillomavirus type 16
  E6V (D32E) aa 26-34
LQTTIHEII

SEQ ID NO: 264 - protein, 11 aa, Human papillomavirus type 16
  E6V (D32E) aa 28-38
TTIHEIILECV

SEQ ID NO: 265 - protein, 11 aa, Human papillomavirus type 16
  E6V (D32E, I34R) aa 28-38
TTIHEIRLECV

SEQ ID NO: 266 - protein, 10 aa, Human papillomavirus type 16
  E6V (D32E) aa 29-38
TIHEIILECV

SEQ ID NO: 267 - protein, 10 aa, Human papillomavirus type 16
  E6V (D32E, I34R) aa 29-38
TIHEIRLECV

SEQ ID NO: 268 - protein, 11 aa, Human papillomavirus type 16
  E6V (D32E) aa 31-41
HEIILECVYCK

SEQ ID NO: 269 - protein, 11 aa, Human papillomavirus type 16
  E6V (D32E, I34R) aa 31-41
HEIRLECVYCK

SEQ ID NO: 270 - protein, 10 aa, Human papillomavirus type 16
  E6V (D32E) aa 32-41
EIILECVYCK

SEQ ID NO: 271 - protein, 8 aa, Human papillomavirus type 16
  E6V (I34R) aa 34-41
RLECVYCK

SEQ ID NO: 272 - protein, 10 aa, Human papillomavirus type 16
  E6V (A68G) aa 60-69
VYRDGNPYGV

SEQ ID NO: 273 - protein, 11 aa, Human papillomavirus type 16
  E6V (H85Y) aa 79-89
KISEYRYYCYS

SEQ ID NO: 274 - protein, 10 aa, Human papillomavirus type 16
  E6V (H85Y) aa 81-90
SEYRYYCYSL

SEQ ID NO: 275 - protein, 10 aa, Human papillomavirus type 16
  E6V (H85Y, L90V) aa 81-90
SEYRYYCYSV

SEQ ID NO: 276 - protein, 11 aa, Human papillomavirus type 16
  E6V (L90V) aa 81-91
SEYRHYCYSVY

SEQ ID NO: 277 - protein, 9 aa, Human papillomavirus type 16
  E6V (L90V) aa 82-90
EYRHYCYSV

SEQ ID NO: 278 - protein, 8 aa, Human papillomavirus type 16
  E6V (H85Y) aa 83-90
YRYYCYSL

SEQ ID NO: 279 - protein, 8 aa, Human papillomavirus type 16
  E6V (L90V) aa 83-90
YRHYCYSV

SEQ ID NO: 280 - protein, 8 aa, Human papillomavirus type 16
  E6V (H85Y, L90V) aa 83-90
YRYYCYSV

SEQ ID NO: 281 - protein, 9 aa, Human papillomavirus type 16
  E6V (L90V) aa 83-91
YRHYCYSVY

SEQ ID NO: 282 - protein, 8 aa, Human papillomavirus type 16
  E6V (L90V) aa 84-91
RHYCYSVY

SEQ ID NO: 283 - protein, 10 aa, Human papillomavirus type 16
  E6V (H85Y) aa 84-93
RYYCYSLYGT

SEQ ID NO: 284 - protein, 11 aa, Human papillomavirus type 16
  E6V (L90V) aa 84-94
RHYCYSVYGTT

SEQ ID NO: 285 - protein, 10 aa, Human papillomavirus type 16
  E6V (L90V) aa 86-95
YCYSVYGTTL

SEQ ID NO: 286 - protein, 9 aa, Human papillomavirus type 16
  E6V (L90V) aa 87-95
CYSVYGTTL

SEQ ID NO: 287 - protein, 10 aa, Human papillomavirus type 16
  E6V (L90V) aa 87-96
CYSVYGTTLE

SEQ ID NO: 288 - protein, 8 aa, Human papillomavirus type 16
  E6V (L90V) aa 88-95
YSVYGTTL

SEQ ID NO: 289 - protein, 11 aa, Human papillomavirus type 16
  E6V (L90V) aa 89-99
SVYGTTLEQQY

SEQ ID NO: 290 - protein, 8 aa, Human papillomavirus type 16
  E6V (L90V) aa 90-97
VYGTTLEQ

SEQ ID NO: 291 - protein, 10 aa, Human papillomavirus type 16
  E6V (L90V) aa 90-99
VYGTTLEQQY

SEQ ID NO: 292 - protein, 8 aa, Human papillomavirus type 16
  E7V (L28F) aa 21-28
DLYCYEQF

SEQ ID NO: 293 - protein, 8 aa, Human papillomavirus type 16
  E7V (S63F) aa 56-63
TFCCKCDF

SEQ ID NO: 294 - protein, 10 aa, Human papillomavirus type 16
  E7V (S63F) aa 56-65
TFCCKCDFTL

SEQ ID NO: 295 - protein, 11 aa, Human papillomavirus type 16
  E7V (S63F) aa 56-66
TFCCKCDFTLR

SEQ ID NO: 296 - protein, 8 aa, Gallus gallus
  Ovalbumin aa 258-265
SIINFEKL

SEQ ID NO: 297 - protein, 9 aa, Homo sapiens
  Cancer/testis antigen 1 NY-ESO-1 aa 127-135
TVSGNILTI

(19) United States
(12) Patent Application Publication
(10) Pub. No.: US 2023/0272014 A1
(43) Pub. Date: Aug. 31, 2023

(54) COMPOSITION OF NANOPARTICLES AS CARRIER FOR HPV-DERIVED IMMUNOGENIC FRAGMENTS

(71) Applicants: Life Science Inkubator Betriebs GmbH & Co. KG, Bonn (DE); Deutsches Krebsforschungszentrum Stiftung des öffentlichen Rechts, Heidelberg (DE)

(72) Inventors: Armin KÜBELBECK, Bonn (DE); Angelika RIEMER, Heidelberg (DE); Sebastian KRUSE, Heidelberg (DE); Eva FEIDT, Bonn (DE); Agnieszka GRABOWSKA, Bonn (DE); Ellen JUNGLAS, Bonn (DE)

(73) Assignees: Life Science Inkubator Betriebs GmbH & Co. KG, Bonn (DE); Deutsches Krebsforschungszentrum Stiftung des öffentlichen Rechts, Heidelberg (DE)

(21) Appl. No.: 17/924,193

(22) PCT Filed: May 12, 2021
(86) PCT No.: PCT/EP2021/062748

§ 371 Date: Nov. 9, 2022

(30) Foreign Application Priority Data
May 12, 2020 (EP) 20174156.8
Sep. 14, 2020 (EP) 20196027.5

(51) Int. Cl. C07K 14/025; A61K 47/69; A61K 39/39; A61K 39/385; A61P 31/20; A61P 35/00

(52) U.S. Cl. CPC C07K 14/025; A61K 47/6929; A61K 39/39; A61K 39/385; A61K 47/6923; A61P 31/20; A61P 35/00; A61K 2039/572

(57) ABSTRACT

The disclosure relates to a composition of nanoparticles as carrier for HPV-derived immunogenic fragments and the use of the composition for medical purposes, in particular for immunoprophylaxis or immunotherapy. The invention also relates to a vaccine containing the composition and/or nanoparticles.

### FIELD OF THE INVENTION

The present invention relates to a composition of nanoparticles as carrier for HPV-derived immunogenic fragments and the use of the composition for medical purposes, in particular for immunoprophylaxis or immunotherapy. The invention also relates to a vaccine containing the composition and/or nanoparticles.

### BACKGROUND OF THE INVENTION

Cervical cancer is one of the most frequent cancer types in the world and commonly induced by human papillomavirus (HPV). Furthermore, HPV infection can cause several other premalignant and malignant conditions and leads to hundreds of thousands deaths per year worldwide. Therefore, effective treatment modalities are urgently needed.

Over 100 individual HPV genotypes have been described. “High-risk” HPV genotypes are associated with the risk of developing a malignant condition. These high-risk genotypes include HPV types 16, 18, 31, 33, 35, 39, 45, 51, 52, 56, 58, 59, 66, 68, 73, and 82, which can lead to cervical cancer and are associated with other mucosal anogenital and head and neck cancers (zur Hausen H., Appl Pathol 1987, 5: 19-24; Bosch F X, et al., J Clin Pathol 55: 244-65; Gillison M L, et al., Vaccine 30 Suppl 5: F34-541-3, 2012). HPV16 and HPV18 are the predominant oncogenic types, cumulatively responsible for over 70-80% of all invasive cervical cancer cases. Accumulating data suggest that both cytotoxic CD8+ T cell and CD4+T helper cell responses play a pivotal role in the control and clearance of HPV infection (Stanley M A, J Reprod Immunol 52: 45-59, 2001; Welters M J, et al., Cancer Res 63: 636-4, 2003; Nakagawa M, et al., J Infect Dis 182: 595-8, 2000; van der Burg S H, et al., Virus Res 89: 275-84, 2002). The HPV proteins E6 and E7 are especially regarded as being crucial for HPV immune escape and malignant progression. E6 and E7 as major transforming proteins are constitutively expressed in both premalignant and advanced lesions, making them ideal targets for immunotherapeutic approaches for HPV-induced malignancies (zur Hausen H., J Natl Cancer Inst 92: 690-8, 2000; Tan S. et al., Curr Cancer Drug Targets 12: 170-84, 2012).

Immunotherapy is an attractive option for treatment of infection and (pre)cancerous conditions. Since CD4+ and CD8+ T cells have been shown to be crucial for the induction and maintenance of cytotoxic T cell responses, and also to be important for direct anti-tumor immunity, immunogenic fragments presented by major histocompatibility complex (MHC) class I and MHC class II are intensively investigated to improve the efficacy of peptide-based HPV immunotherapy, such as HPV vaccines. Each human being expresses three to six MHC class I and at least as many MHC class II molecules, also called human leukocyte antigen (HLA) molecules. To date, more than 3000 variants of human MHC class I and 1000 variants of MHC class II have been characterized (Robinson J, et al., Nucleic Acids Res 37: D1013-D1017, 2009). Thus, the use of immunogenic fragments having the ability to bind to a wide variety of different HLA molecules for effective immunoprophylaxis and immunotherapy is desired. Hence, to focus the cytotoxic T cell response on E6 and/or E7 proteins, it is crucial to define the immunogenic fragments presented on the HLA molecules of malignant cells.

A key advance in coping with HPV infection and related conditions has been the development of virus-like particle (VLP) prophylactic vaccines. To date, the three HPV vaccines available on the market comprise the recombinantly produced HPV16- and HPV18-derived major capsid protein L1. Cervarix® (GlaxoSmithKline) comprises recombinantly produced HPV16 and HPV18 VLP and is formulated with the immunostimulant 3-O-desacyl-4′-monophosphoryl lipid A (3D MPL, also known as MPL) and aluminium hydroxide salt. Gardasil® and Gardasil®9 (Merck Sharp & Dohme) contain recombinantly produced HPV16 and HPV18 VLP and are formulated with amorphous aluminium hydroxyphosphate sulphate salt. While Gardasil® also contains VLP of HPV6 and HPV11, Gardasil®9 additionally comprises VLP of HPV31, HPV33, HPV45, HPV52, and HPV58. For these approved vaccines, specific protection against infection with oncogenic types HPV16 and HPV18 and associated precancerous lesions has been demonstrated in randomized clinical trials.

The synthetic DNA vaccine VGX-3100, targeting HPV16 and HPV18 E6 and E7 proteins, is currently investigated in clinical trials for the possible use in immunotherapy of HPV16 and HPV18 infection and precancerous lesions of the cervix (phase III) and vulva (phase II).

Several immunogenic fragments of the E6 and E7 proteins of HPV suitable for the treatment of HPV-related conditions are known in the art.

For instance, the safety and efficacy of the HPV vaccine DPX-E7, comprising the synthetic immunogenic fragment E7₁₁₋₁₉ of HPV16, is currently investigated in a phase Ib/II clinical trial as a possible treatment option for HPV or HPV-related head and neck, cervical or anal cancer.

WO 2015/086354 A2 relates to novel amino acid sequences of peptides derived from HPV16 that are able to bind to MHC class II, and elicit an immune response (columns 4/5, tables A and A1, SEQ ID Nos: 4 and 7). Furthermore, said peptides are suggested for the use in pharmaceutical products, such as vaccines.

WO 2018/085751 A1 relates to proteins or polypeptides derived from a HPV E6 protein or polypeptide (p. 30, table 1, SEQ ID NOs: 4, 5, 7) and a HPV E7 protein or polypeptide (p. 30, table 1, SEQ ID NOs: 8, 10, 12). Moreover, a method of inhibiting HPV infection or HPV-associated cancer in a subject by administering a composition comprising the protein or peptide to the subject is described.

Since antigens, when injected alone, are usually ignored by antigen-presenting cells, cleared rapidly and, thus, do not induce an immune response, its administration in combination with a suitable adjuvant is required. Thus, there is a high demand on providing a carrier system that ensures adequate delivery of HPV-derived immunogenic fragments and, hence, proper presentation of those immunogenic fragments bound to MHC class I and MHC class II to CD4+ and CD8+ T cells.

Nanomedicine and nano-delivery systems are a relatively new but rapidly developing science where materials in the nanoscale range are employed to serve as means of diagnostic or therapeutic tools or to deliver therapeutic agents to specific targeted sites in the body. In particular nanoparticles are known for delivery of chemotherapeutic agents, biological agents, immunotherapeutic agents and as vaccines in the immunotherapy.

Functionalized nanoparticles are well-known as drug-delivery systems, e.g. as carrier for antigens. The literature describes in particular carrier systems in which the antigens are either encapsulated or bound to the surface of the nanoparticles.

WO 2006/037979 A2 describes gold nanoparticles (GNPs) comprising adjuvants and antigens, such as tumor and pathogen antigens, and their use in a range of applications such as for the treatment of cancer and infectious diseases. Also disclosed are immunogenic structures based on nanoparticles or antibodies with carbohydrate ligands, and their use for therapeutic and prophylactic purposes, and for the isolation and detection of antibodies directed against the carbohydrate structures.

WO 2013/034741 A1 and WO 2013/034726 A1 relate to nanoparticles having an epitopic peptide bound via a linker and which find use as vaccines, e.g. in the prophylactic or therapeutic treatment of a tumor in a mammalian subject.

WO 2011/154711 A1 describes glycated gold nanoparticles that act as carriers for delivery of peptides such as insulin.

WO 2010/006753 A2 discloses monodisperse nanoparticles of silicon dioxide with at least one antigen attached to their surface. The nanoparticles are used for the immunoprophylaxis or immunotherapy of cancer.

Although many nanoparticles have been investigated and developed in particular in the context of treating cancer there is still a high demand on providing substances with improved characteristics, in particular in terms of its adjuvant effects. Furthermore there is a need to provide nanoparticles as a flexible and convenient system to present agents of pharmacological relevance, such as HPV-derived immunogenic fragments, to the immune system of the body.

### SUMMARY OF THE INVENTION

It is thus objective of the present invention to provide improved nanoparticles and compositions as carrier for HPV-derived immunogenic fragments comprising them in particular for use in the immunoprophylaxis or immunotherapy. It is furthermore an objective of the invention to provide improved HPV vaccine compositions for use in the prevention and treatment of HPV-related conditions.

These objectives are solved by providing a composition comprising nanoparticles with silicon dioxide and functional groups on the surface, which are loaded with pharmaceutically acceptable compounds comprising HPV-derived immunogenic fragments. Alternatively, the nanoparticles are loaded with pharmaceutically acceptable compounds comprising HPV-derived immunogenic fragments and poly(I:C) or any derivatives thereof. The pharmaceutically acceptable compounds (poly(I:C) and HPV-derived immunogenic fragments) represent the payload of the nanoparticles. The nanoparticles have a particle size below 150 nm. The functional groups on the surface of the nanoparticles are suitable for carrying and/or stabilizing negative and positive charges of such compounds. The composition has a zeta potential of at least ±15 mV.

Poly(I:C) is a synthetic dsRNA that can activate multiple elements of the host defense in a pattern that parallels that of a viral infection. Derivatives of poly(I:C) are for example polyl:polyC₁₂U and poly-ICLC.

It was found that compositions according to the invention show an immunomodulatory efficacy for immunoprophylaxis and immunotherapy of HPV-related conditions.

Moreover, with a composition according to the invention one fundamental problem of nanoparticles for drug delivery is overcome, which is the lack of stability of the colloidal suspension even under physiological conditions.

In aqueous systems, surface charges essentially ensure the stability of colloidal systems. These charges can be positive or negative. To substantially reduce or even avoid agglomeration of the particles it is important that there are enough functionalities of the same charge, which leads to electrostatic repulsion. If the charge of the colloidal carrier system is compensated by the adsorption of the oppositely charged molecules, agglomerates will be formed and the colloidal system collapses.

The agglomerates have significantly larger particle diameters, which jeopardizes an effective transport of these large particles within the body and leads to a less effective immunomodulatory efficacy.

The composition according to the invention with nanoparticles having a particle size of below 150 nm and a zeta potential of at least ±15 mV ensures that the composition is sufficiently stable. In particular under physiological conditions, the nanoparticles dispersed therein can effectively be transported to their site of main activity within the body. The compositions according to the invention thus allow the improved transport of the nanoparticles into the lymphatic system, in particular from the administration site to the lymph nodes in which dendritic cells are located.

When administered by subcutaneous injection the particles according to the invention are too large to enter the blood circulation, hence their fast elimination as well as severe systemic adverse effects are essentially avoided. At the same time, they are small enough to penetrate lymphatic vessels and to enter naïve dendritic cells, located in lymph nodes, by phagocytosis. The same applies when the particles are administered intradermally, intraperitoneally or intramuscularly. It is thus preferred to administer the particles parenterally with the exception of intravenous or intraarterial administration.

The inventors have found that the particles in the composition according to the invention can carry a high number of pharmaceutically acceptable compounds on the surface. The total amount of the compounds can be up to 5% by weight with regard to the surface of the nanoparticle in nm² (surface loading density). The suitability of the nanoparticles to carry huge amounts of compounds is particularly important when high doses of such compounds (e.g. antigens or drug substances) are to be delivered.

The compositions according to the invention comprise nanoparticles with silicon dioxide and functional groups on the surface. The functional groups can be directly or indirectly connected to the surface of the nanoparticle. In a preferred embodiment of the invention the functional groups are connected to the surface of the nanoparticle via a linker (hereinafter linker compound L). The linker compound L can be connected to the surface of the nanoparticles by any way, in particular by covalent or adsorptive bond, most preferred by covalent bond.

In a particularly preferred embodiment the linker compound L comprises at least one functional group selected from the group consisting of carboxyl (—COOH) or carboxylate (—COO—) group and/or at least one functional group selected from the group consisting of guanidino-group (—NHC(═NH)NH₂ or —NHC(═NH₂⁺)NH₂) or amino-group (—NH₂ or —NH₃⁺). Preferably, it comprises both, a carboxyl (—COOH) or carboxylate (—COO—) group and at least one group selected from the group consisting of guanidino-group (—NHC(═NH)NH₂ or —NHC(═NH₂⁺)NH₂) or amino-group (—NH₂ or —NH₃⁺). Most preferred is that the linker compound L comprises a carboxyl (—COOH) or carboxylate (—COO—) group and at least one guanidino-group (—NHC(═NH)NH₂ or —NHC(═NH₂+)NH₂).

Such a linker compound L allows the adsorptive binding of anionic or cationic and also of nonpolar pharmaceutically acceptable compounds (such as e.g. hydrophobic peptides). Therewith the linker provides a simple coupling of HPV-derived immunogenic fragments and alternatively in combination with poly(I:C) to the surface of the nanoparticles.

### DETAILED DESCRIPTION OF THE INVENTION

The composition of the present invention contains nanoparticles which are loaded with one or more human papilloma virus (HPV)-derived immunogenic fragments or a variant thereof.

An “immunogenic fragment” according to the present invention is a peptide that is derived from a pathogenic antigen or epitope of a high-risk HPV genotype and able to induce an immune response in a patient. An immunogenic fragment of the invention is artificially produced.

A “peptide” according to the present invention is composed of any number of amino acids of any type, preferably naturally occurring amino acids, which preferably are linked by peptide bonds. The peptides can also exhibit posttranslational modifications, as e.g. phosphorylations, glycosylations, lipidations (like myristoylation or palmitoylation), citrullinations, acetylations (of lysine), hydroxylations (of proline or lysine).

An “antigen” according to the invention is a structure, which is capable of inducing a cellular or humoral immune response. Antigens are preferably proteinogenic, i.e. they are proteins, polypeptides or peptides. In principle, they can have any size, origin and molecular weight. They contain at least one antigenic determinant or an antigenic epitope.

In an alternative embodiment the antigen is a nucleic acids per se or is encoded by a nucleic acid, which, after transport into the nucleus of antigen-presenting cells, are translated into the proteinogenic antigen which is then presented by MHC molecules.

The nucleic acids can be single- and double-stranded DNA or RNA or oligonucleotides. The nucleic acids may also be a constituent of complexes with lipids, carbohydrates, proteins or peptides.

An “epitope” according to the invention, also known as antigenic determinant, is the part of an antigen that is recognized by the immune system, specifically by antibodies, B cells, or T cells. T cell epitopes are presented on the surface of an antigen-presenting cell, where they are bound to MHC molecules. In humans, professional antigen-presenting cells are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids.

A “variant” according to the present invention is an immunogenic fragment that is at least 75% identical to an immunogenic fragment derived from a pathogenic antigen or epitope of a high-risk HPV genotype.

The zeta potential of a nanoparticle is a commonly used parameter known to the skilled person to characterize the surface charge property of nanoparticles. It reflects the electrical potential of particles. To avoid agglomeration of the particles it is important that there are enough functionalities of the same charge which repel each other. Agglomerates cause the colloidal system to collapse. The resulting agglomerates have significantly larger particle diameters than the individual particles and therefore the desired transport mechanism via the fenestrated endothelium of the lymphatic vessels is no longer guaranteed. It is accepted as a measure for the stability of colloidal systems.

Current characterization methodologies are based on ensemble measurements (e.g. phase analysis light scattering, Doppler velocimetry, streaming potentiometry) that measure the average electrophoretic mobility of particles in suspension (Sci. Rep. 12 Dec. 2017; 7(1): 17479, PMCID: PMC5727177). It is particularly preferred to measure the zeta potential of the composition according to the invention with Dynamic Light Scattering (DLS) (ZetaSizer Nano ZS, Malvern Instruments, UK).

The zeta potential of the composition according to the present invention has a value of at least ±15 mV, preferred ±30 mV, more preferred ±60 mV and most preferred between ±25 and ±40 mV. Therewith the composition, which is desirably a colloidal suspension, is stabilized, which means that the collapse of the system is essentially avoided.

The compositions according to the invention contain nanoparticles with a particle size below 150 nm, preferably 100 nm or less. More preferred are nanoparticles with a particle size of 50 nm or less, most preferred with a particle size between 20 and 30 nm. The particle size defined herein should be interpreted in such a way that a random distribution over the entire range is not present, but instead a defined particle size within the range is selected, of which the standard deviation is a maximum of 15%, preferably a maximum of 10%, wherein the standard deviation always relates to the local maximum in case of a bi- or multimodal distribution.

An indicator for the particle size of nanoparticles is the Z-average diameter. The Z-average diameter measured in dynamic light scattering is a parameter also known as the cumulant mean. It is the primary and most reliable parameter produced by the technique. The Z-average diameter is typically used in a quality control setting according to ISO 22412:2017. Dynamic light scattering techniques will give an intensity weighted distribution, where the contribution of each particle in the distribution relates to the intensity of light scattered by the particle. It is preferred to measure the intensity weighted distributions with a ZetaSizer Nano ZS (Malvern Instruments, UK).

In a preferred embodiment the Z-average diameter of the nanoparticles according to the invention is in the range of ≥5 and <150 nm, preferably ≥15 and ≤60 nm, more preferably ≥20 and ≤40 nm and still more preferably between 20 and 30 nm, measured according to ISO 22412:2017.

The particle size of the nanoparticles and the stability of the composition according to the invention are furthermore confirmed by means of filtration with a sterile filter with maximum 0.2 μm pore size. However, this is practical only for nanoparticles with a diameter less than 50 nm.

The stability of the composition of the invention can be demonstrated by its polydispersity index (PDI). The PDI in general is an indicator for the uniformity of a system; in the context of the invention for the uniformity and stability of the colloidal nanoparticle suspension. The PDI reflects the nanoparticle size distribution. Samples with a wider range of particle sizes have higher PDI, while samples consisting of evenly sized particles have lower PDI. The skilled person is aware of methods and instruments for the measurement of the PDI, in particular a ZetaSizer Nano ZS (Malvern Instruments, UK).

A PDI greater than 0.7 indicates that the sample has a broad size distribution. A PDI below 0.1 is considered to be monodisperse. The various size distribution algorithms work with data that falls between these two extremes. The calculations for these parameters are defined in the ISO standard document 13321:1996 E and ISO 22412:2008.

The compositions according to the invention show a PDI between 0 and 0.32, preferably between 0.1 and 0.3, more preferably between 0.1 and 0.2, most preferred less than 0.1. The compositions according to the invention have a PDI less than 0.1 and are preferably monodisperse.

In a preferred embodiment the Z-average diameter of the nanoparticles according to the invention is in the range of ≥20 and ≤40 nm and a PDI between 0.1 and 0.30 and a zeta potential between ±20 and ±40 mV.

In a more preferred embodiment the Z-average diameter of the nanoparticles according to the invention is in the range of 20 and 30 nm and a PDI between 0.1 and 0.2 and a zeta potential between ±25 and ±40 mV.

In a most preferred embodiment the Z-average diameter of the nanoparticles according to the invention is in the range of 20 and 30 nm and a PDI less than 0.1 and a zeta potential between ±25 and ±40 mV.

It is also possible to measure the particle size of the loaded and unloaded particles by TEM or SEM.

As used herein, the term “transmission electron microscopy (TEM)” refers to a microscopy technique whereby a beam of electrons is transmitted through an ultra thin specimen, interacting with the specimen as it passes through it. An image is formed from the electrons transmitted through the specimen, magnified and focused by an objective lens and appears on an imaging screen, a fluorescent screen in most TEMs, plus a monitor, or on a layer of photographic film, or to be detected by a sensor such as a CCD camera.

As used herein, the term “scanning electron microscope (SEM)” refers to a type of electron microscope that produces images of a sample by scanning the surface with a focused beam of electrons. The electrons interact with atoms in the sample, producing various signals that contain information about the surface topography and composition of the sample. The electron beam is scanned in a raster scan pattern, and the position of the beam is combined with the intensity of the detected signal to produce an image.

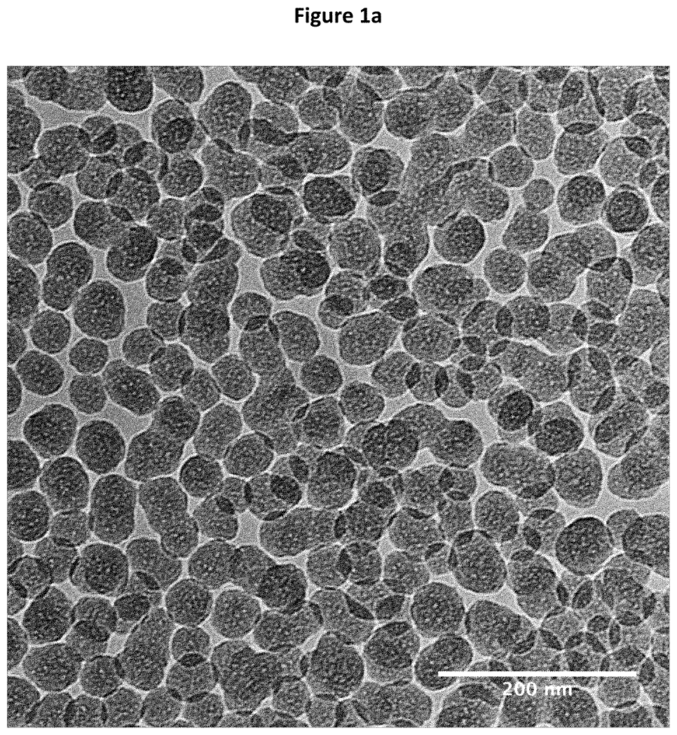

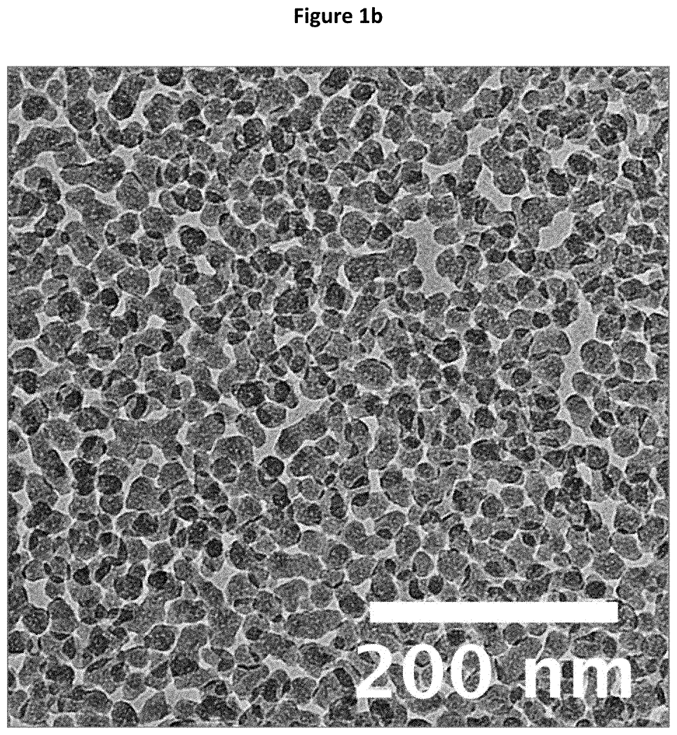

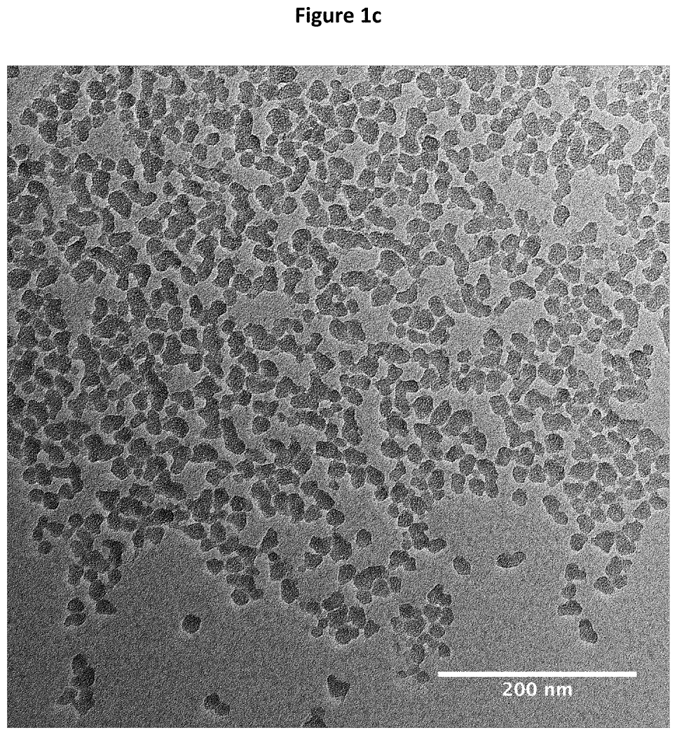

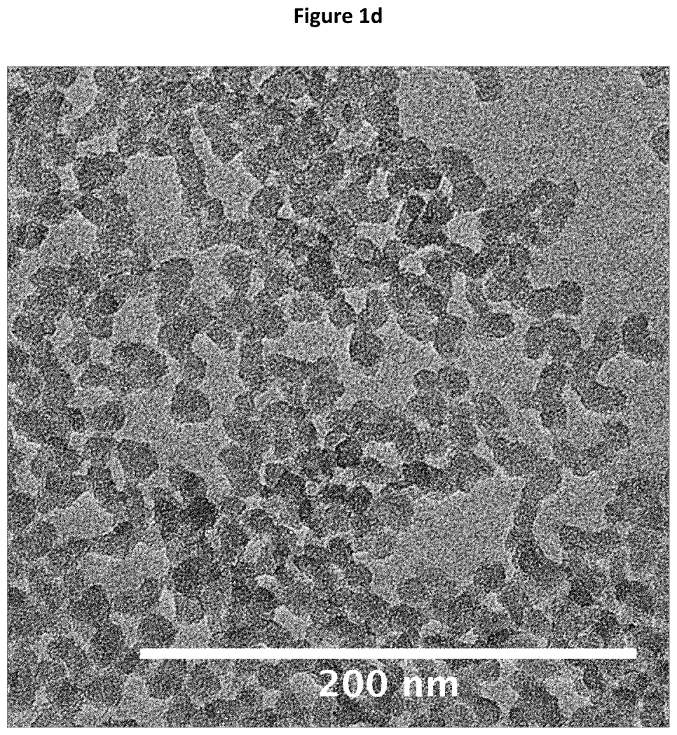

In FIG. 1a to 1d show TEM images of the following SiO₂ particles:

FIG. 1a shows a TEM image of SiO₂ nanoparticles with a particle size of 68 nm

FIG. 1b shows a TEM image of SiO₂ nanoparticles with a particle size of 40 nm

FIG. 1c shows a TEM image of SiO₂ nanoparticles with a particle size of 25 nm

FIG. 1d shows a TEM image of SiO₂ nanoparticles with a particle size of 15 nm

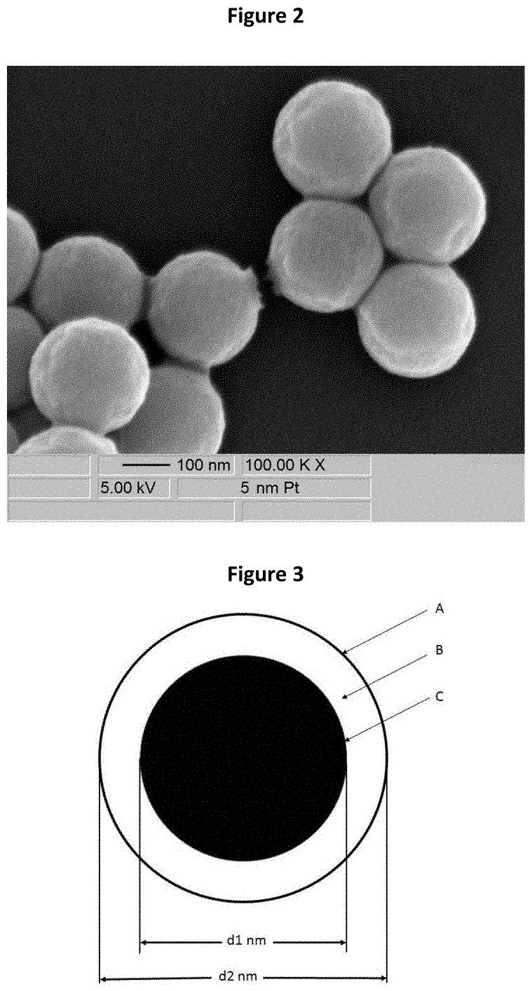

FIG. 2 shows a SEM of SiO₂ nanoparticles with a particle size of 150 nm.

In connection with the present invention, a “nanoparticle” is taken to mean a particulate binding matrix which has functionalities on its surface which function as recognition points for pharmaceutically acceptable compounds, e.g. antigens ultimately to be bound or adsorbed. The surface here encompasses all areas, i.e. besides the outer surface, also the inner surface of cavities (pores) in the particle. The functionalities may be directly or indirectly bound to the surface.

Any immunogenic fragment derived from a pathogenic antigen or epitope associated with any of the diseases or conditions provided herein can be used in the compositions and methods described herein. These include immunogenic fragments derived from a pathogenic antigen or epitope associated with cancer, infections or infectious disease. Preferred immunogenic fragments derived from a pathogenic antigen or epitope are those which are associated with a HPV infection, preferably caused by high-risk HPV genotypes.

So-called high-risk HPV genotypes are associated with the risk of developing a malignant condition, such as HPV types 16, 18, 31, 33, 35, 39, 45, 51, 52, 56, 58, 59, 66, 68, 73, and 82. Moreover, the HPV proteins E6 and E7 are especially regarded as being crucial for HPV immune escape and malignant progression.

Thus, in a preferred embodiment of the invention the immunogenic fragment bound to the nanoparticles is derived from a high-risk HPV genotype, preferably selected from HPV types 16, 18, 31, 33, 35, 39, 45, 51, 52, 56, 58, 59, 66, 68, 73, and 82.

In a more preferred embodiment of the present invention the immunogenic fragment is derived from the E6 and/or E7 proteins of HPV16 and/or HPV18 (SEQ ID NOs: 1-4), preferably selected from SEQ ID NOs: 5-257.

TABLE 1
Sequence listing

| SEQ | | | | |
|---|---|---|---|---|
| ID | HPV | protein/ | | |
| NO | type | variant | amino acids | sequence |
| 1 | HPV16 | E6 | 1-158 | MHQKRTAMFQDPQERPRKLPQLCTELQTTI |
| | | | | HDIILECVYCKQQLLRREVYDFAFRDLCIVYR |
| | | | | DGNPYAVCDKCLKFYSKISEYRHYCYSLYG |
| | | | | TTLEQQYNKPLCDLLIRCINCQKPLCPEEKQ |
| | | | | RHLDKKQRFHNIRGRWTGRCMSCCRSSRT |
| | | | | RRETQL |
| 2 | HPV16 | E7 | 1-98 | MHGDTPTLHEYMLDLQPETTDLYCYEQLND |
| | | | | SSEEEDEIDGPAGQAEPDRAHYNIVTFCCK |
| | | | | CDSTLRLCVQSTHVDIRTLEDLLMGTLGIVC |
| | | | | PICSQKP |
| 3 | HPV18 | E6 | 1-158 | MARFEDPTRRPYKLPDLCTELNTSLQDIEIT |
| | | | | CVYCKTVLELTEVFEFAFKDLFVVYRDSIPH |
| | | | | AACHKCIDFYSRIRELRHYSDSVYGDTLEKL |
| | | | | TNTGLYNLLIRCLRCQKPLNPAEKLRHLNEK |
| | | | | RRFHNIAGHYRGQCHSCCNRARQERLQRR |
| | | | | RETQV |
| 4 | HPV18 | E7 | 1-105 | MHGPKATLQDIVLHLEPQNEIPVDLLCHEQL |
| | | | | SDSEEENDEIDGVNHQHLPARRAEPQRHTM |
| | | | | LCMCCKCEARIKLVVESSADDLRAFQQLFLN |
| | | | | TLSFVCPWCASQQ |
| 5 | HPV 16 | E6 | 1-30 | MHQKRTAMFQDPQERPRKLPQLCTELQTTI |
| 6 | HPV 16 | E6 | 7-15 | AMFQDPQER |
| 7 | HPV 16 | E6 | 8-18 | MFQDPQERPRK |
| 8 | HPV 16 | E6 | 9-18 | FQDPQERPRK |
| 9 | HPV 16 | E6 | 11-19 | DPQERPRKL |
| 10 | HPV 16 | E6 | 11-32 | DPQERPRKLPQLCTELQTTIHD |
| 11 | HPV 16 | E6 | 13-22 | QERPRKLPQL |
| 12 | HPV 16 | E6 | 15-22 | RPRKLPQL |
| 13 | HPV 16 | E6 | 15-23 | RPRKLPQLC |
| 14 | HPV 16 | E6 | 15-24 | RPRKLPQLCT |
| 15 | HPV 16 | E6 | 15-25 | RPRKLPQLCTE |
| 16 | HPV 16 | E6 | 18-26 | KLPQLCTEL |
| 17 | HPV 16 | E6 | 18-28 | KLPQLCTELQT |
| 18 | HPV 16 | E6 | 19-26 | LPQLCTEL |
| 19 | HPV 16 | E6 | 19-28 | LPQLCTELQT |
| 20 | HPV 16 | E6 | 23-31 | CTELQTTIH |
| 21 | HPV 16 | E6 | 25-33 | ELQTTIHDI |
| 22 | HPV 16 | E6 | 26-34 | LQTTIHDII |
| 23 | HPV 16 | E6 | 28-38 | TTIHDIILECV |
| 24 | HPV 16 | E6 | 29-37 | TIHDIILEC |
| 25 | HPV 16 | E6 | 29-38 | TIHDIILECV |
| 26 | HPV 16 | E6 | 29-39 | TIHDIILECVY |
| 27 | HPV 16 | E6 | 30-39 | IHDIILECVY |
| 28 | HPV 16 | E6 | 31-38 | HDIILECV |
| 29 | HPV 16 | E6 | 31-39 | HDIILECVY |
| 30 | HPV 16 | E6 | 31-41 | HDIILECVYCK |
| 31 | HPV 16 | E6 | 32-41 | DIILECVYCK |
| 32 | HPV 16 | E6 | 33-41 | IILECVYCK |
| 33 | HPV 16 | E6 | 34-41 | ILECVYCK |
| 34 | HPV 16 | E6 | 34-44 | ILECVYCKQQL |
| 35 | HPV 16 | E6 | 35-44 | LECVYCKQQL |
| 36 | HPV 16 | E6 | 37-46 | CVYCKQQLLR |
| 37 | HPV 16 | E6 | 37-54 | CVYCKQQLLRREVYDFAF |
| 38 | HPV 16 | E6 | 37-68 | CVYCKQQLLRREVYDFAFRDLCIVYRDGNP |
| | | | | YA |
| 39 | HPV 16 | E6 | 38-45 | VYCKQQLL |
| 40 | HPV 16 | E6 | 38-46 | VYCKQQLLR |
| 41 | HPV 16 | E6 | 38-47 | VYCKQQLLRR |
| 42 | HPV 16 | E6 | 40-50 | CKQQLLRREVY |
| 43 | HPV 16 | E6 | 41-50 | KQQLLRREVY |
| 44 | HPV 16 | E6 | 42-50 | QQLLRREVY |
| 45 | HPV 16 | E6 | 42-52 | QQLLRREVYDF |
| 46 | HPV 16 | E6 | 43-52 | QLLRREVYDF |
| 47 | HPV 16 | E6 | 43-57 | QLLRREVYDFAFRDL |
| 48 | HPV 16 | E6 | 44-52 | LLRREVYDF |
| 49 | HPV 16 | E6 | 44-54 | LLRREVYDFAF |
| 50 | HPV 16 | E6 | 45-68 | LRREVYDFAFRDLCIVYRDGNPYA |
| 51 | HPV 16 | E6 | 47-54 | REVYDFAF |
| 52 | HPV 16 | E6 | 48-55 | EVYDFAFR |
| 53 | HPV 16 | E6 | 48-57 | EVYDFAFRDL |
| 54 | HPV 16 | E6 | 49-57 | VYDFAFRDL |
| 55 | HPV 16 | E6 | 49-58 | VYDFAFRDLC |
| 56 | HPV 16 | E6 | 49-59 | VYDFAFRDLCI |
| 57 | HPV 16 | E6 | 50-59 | YDFAFRDLCI |
| 58 | HPV 16 | E6 | 51-59 | DFAFRDLCI |
| 59 | HPV 16 | E6 | 52-60 | FAFRDLCIV |
| 60 | HPV 16 | E6 | 52-61 | FAFRDLCIVY |
| 61 | HPV 16 | E6 | 52-62 | FAFRDLCIVYR |
| 62 | HPV 16 | E6 | 53-61 | AFRDLCIVY |
| 63 | HPV 16 | E6 | 53-62 | AFRDLCIVYR |
| 64 | HPV 16 | E6 | 54-68 | FRDLCIVYRDGNPYA |
| 65 | HPV 16 | E6 | 55-86 | RDLCIVYRDGNPYAVCDKCLKFYSKISEYRH |
| | | | | Y |
| 66 | HPV 16 | E6 | 57-67 | LCIVYRDGNPY |
| 67 | HPV 16 | E6 | 58-67 | CIVYRDGNPY |
| 68 | HPV 16 | E6 | 59-67 | IVYRDGNPY |
| 69 | HPV 16 | E6 | 59-68 | IVYRDGNPYA |
| 70 | HPV 16 | E6 | 59-69 | IVYRDGNPYAV |
| 71 | HPV 16 | E6 | 60-67 | VYRDGNPY |
| 72 | HPV 16 | E6 | 60-69 | VYRDGNPYAV |
| 73 | HPV 16 | E6 | 61-82 | YRDGNPYAVCDKCLKFYSKISE |
| 74 | HPV 16 | E6 | 65-75 | NPYAVCDKCLK |
| 75 | HPV 16 | E6 | 66-74 | PYAVCDKCL |
| 76 | HPV 16 | E6 | 66-75 | PYAVCDKCLK |
| 77 | HPV 16 | E6 | 66-76 | PYAVCDKCLKF |
| 78 | HPV 16 | E6 | 67-75 | YAVCDKCLK |
| 79 | HPV 16 | E6 | 67-76 | YAVCDKCLKF |
| 80 | HPV 16 | E6 | 67-77 | YAVCDKCLKFY |
| 81 | HPV 16 | E6 | 68-75 | AVCDKCLK |
| 82 | HPV 16 | E6 | 68-76 | AVCDKCLKF |
| 83 | HPV 16 | E6 | 68-77 | AVCDKCLKFY |
| 84 | HPV 16 | E6 | 68-78 | AVCDKCLKFYS |
| 85 | HPV 16 | E6 | 69-79 | VCDKCLKFYSK |
| 86 | HPV 16 | E6 | 71-78 | DKCLKFYS |
| 87 | HPV 16 | E6 | 72-80 | KCLKFYSKI |
| 88 | HPV 16 | E6 | 73-83 | CLKFYSKISEY |
| 89 | HPV 16 | E6 | 73-105 | CLKFYSKISEYRHYCYSLYGTTLEQQYNKPL |
| | | | | CD |
| 90 | HPV 16 | E6 | 74-83 | LKFYSKISEY |
| 91 | HPV 16 | E6 | 74-88 | LKFYSKISEYRHYCY |
| 92 | HPV 16 | E6 | 75-83 | KFYSKISEY |
| 93 | HPV 16 | E6 | 75-84 | KFYSKISEYR |
| 94 | HPV 16 | E6 | 75-85 | KFYSKISEYRH |
| 95 | HPV 16 | E6 | 76-83 | FYSKISEY |
| 96 | HPV 16 | E6 | 76-85 | FYSKISEYRH |
| 97 | HPV 16 | E6 | 76-86 | FYSKISEYRHY |
| 98 | HPV 16 | E6 | 77-86 | YSKISEYRHY |
| 99 | HPV 16 | E6 | 78-86 | SKISEYRHY |
| 100 | HPV 16 | E6 | 78-88 | SKISEYRHYCY |
| 101 | HPV 16 | E6 | 79-86 | KISEYRHY |
| 102 | HPV 16 | E6 | 79-87 | KISEYRHYC |
| 103 | HPV 16 | E6 | 79-88 | KISEYRHYCY |
| 104 | HPV 16 | E6 | 80-88 | ISEYRHYCY |
| 105 | HPV 16 | E6 | 81-88 | SEYRHYCY |
| 106 | HPV 16 | E6 | 81-90 | SEYRHYCYSL |
| 107 | HPV 16 | E6 | 81-91 | SEYRHYCYSLY |
| 108 | HPV 16 | E6 | 82-90 | EYRHYCYSL |
| 109 | HPV 16 | E6 | 82-91 | EYRHYCYSLY |
| 110 | HPV 16 | E6 | 83-90 | YRHYCYSL |
| 111 | HPV 16 | E6 | 83-91 | YRHYCYSLY |
| 112 | HPV 16 | E6 | 84-91 | RHYCYSLY |
| 113 | HPV 16 | E6 | 84-94 | RHYCYSLYGTT |
| 114 | HPV 16 | E6 | 85-95 | HYCYSLYGTTL |
| 115 | HPV 16 | E6 | 86-96 | YCYSLYGTTLE |
| 116 | HPV 16 | E6 | 87-95 | CYSLYGTTL |
| 117 | HPV 16 | E6 | 87-96 | CYSLYGTTLE |
| 118 | HPV 16 | E6 | 88-95 | YSLYGTTL |
| 119 | HPV 16 | E6 | 89-99 | SLYGTTLEQQY |
| 120 | HPV 16 | E6 | 90-99 | LYGTTLEQQY |
| 121 | HPV 16 | E6 | 91-100 | YGTTLEQQYN |
| 122 | HPV 16 | E6 | 91-101 | YGTTLEQQYNK |
| 123 | HPV 16 | E6 | 91-112 | YGTTLEQQYNKPLCDLLIRCIN |
| 124 | HPV 16 | E6 | 92-99 | GTTLEQQY |
| 125 | HPV 16 | E6 | 92-101 | GTTLEQQYNK |
| 126 | HPV 16 | E6 | 93-101 | TTLEQQYNK |
| 127 | HPV 16 | E6 | 94-101 | TLEQQYNK |
| 128 | HPV 16 | E6 | 95-103 | LEQQYNKPL |
| 129 | HPV 16 | E6 | 97-106 | QQYNKPLCDL |
| 130 | HPV 16 | E6 | 98-106 | QYNKPLCDL |
| 131 | HPV 16 | E6 | 98-107 | QYNKPLCDLL |
| 132 | HPV 16 | E6 | 98-108 | QYNKPLCDLLI |
| 133 | HPV 16 | E6 | 99-106 | YNKPLCDL |
| 134 | HPV 16 | E6 | 101-108 | KPLCDLLI |
| 135 | HPV 16 | E6 | 101-111 | KPLCDLLIRCI |
| 136 | HPV 16 | E6 | 101-122 | KPLCDLLIRCINCQKPLCPEEK |
| 137 | HPV 16 | E6 | 105-115 | DLLIRCINCQK |
| 138 | HPV 16 | E6 | 106-115 | LLIRCINCQK |
| 139 | HPV 16 | E6 | 107-115 | LIRCINCQK |
| 140 | HPV 16 | E6 | 107-117 | LIRCINCQKPL |
| 141 | HPV 16 | E6 | 109-119 | RCINCQKPLCP |
| 142 | HPV 16 | E6 | 113-121 | CQKPLCPEE |
| 143 | HPV 16 | E6 | 118-126 | CPEEKQRHL |
| 144 | HPV 16 | E6 | 121-142 | EKQRHLDKKQRFHNIRGRWTGR |
| 145 | HPV 16 | E6 | 122-132 | KQRHLDKKQRF |
| 146 | HPV 16 | E6 | 125-133 | HLDKKQRFH |
| 147 | HPV 16 | E6 | 125-135 | HLDKKQRFHNI |
| 148 | HPV 16 | E6 | 127-134 | DKKQRFHN |
| 149 | HPV 16 | E6 | 127-135 | DKKQRFHNI |
| 150 | HPV 16 | E6 | 127-141 | DKKQRFHNIRGRWTG |
| 151 | HPV 16 | E6 | 128-135 | KKQRFHNI |
| 152 | HPV 16 | E6 | 128-136 | KKQRFHNIR |
| 153 | HPV 16 | E6 | 129-138 | KQRFHNIRGR |
| 154 | HPV 16 | E6 | 129-139 | KQRFHNIRGRW |
| 155 | HPV 16 | E6 | 131-139 | RFHNIRGRW |
| 156 | HPV 16 | E6 | 134-144 | NIRGRWTGRCM |
| 157 | HPV 16 | E6 | 136-144 | RGRWTGRCM |
| 158 | HPV 16 | E6 | 137-146 | GRWTGRCMSC |
| 159 | HPV 16 | E6 | 139-148 | WTGRCMSCCR |
| 160 | HPV 16 | E6 | 142-151 | RCMSCCRSSR |
| 161 | HPV 16 | E6 | 148-158 | RSSRTRRETQL |
| 162 | HPV 16 | E6 | 149-158 | SSRTRRETQL |
| 163 | HPV 16 | E6 | 151-158 | RTRRETQL |
| 164 | HPV 16 | E7 | 1-12 | MHGDTPTLHEYM |
| 165 | HPV 16 | E7 | 2-11 | HGDTPTLHEY |
| 166 | HPV 16 | E7 | 4-11 | DTPTLHEY |
| 167 | HPV 16 | E7 | 5-12 | TPTLHEYM |
| 168 | HPV 16 | E7 | 5-13 | TPTLHEYML |
| 169 | HPV 16 | E7 | 5-18 | TPTLHEYMLDLQPE |
| 170 | HPV 16 | E7 | 7-15 | TLHEYMLDL |
| 171 | HPV 16 | E7 | 7-17 | TLHEYMLDLQP |
| 172 | HPV 16 | E7 | 7-27 | TLHEYMLDLQPETTDLYCYEQ |
| 173 | HPV 16 | E7 | 11-18 | YMLDLQPE |
| 174 | HPV 16 | E7 | 11-19 | YMLDLQPET |
| 175 | HPV 16 | E7 | 11-20 | YMLDLQPETT |
| 176 | HPV 16 | E7 | 11-21 | YMLDLQPETTD |
| 177 | HPV 16 | E7 | 11-26 | YMLDLQPETTDLYCYE |
| 178 | HPV 16 | E7 | 12-19 | MLDLQPET |
| 179 | HPV 16 | E7 | 12-20 | MLDLQPETT |
| 180 | HPV 16 | E7 | 12-26 | MLDLQPETTDLYCYE |
| 181 | HPV 16 | E7 | 14-23 | DLQPETTDLY |
| 182 | HPV 16 | E7 | 15-23 | LQPETTDLY |
| 183 | HPV 16 | E7 | 15-24 | LQPETTDLYC |
| 184 | HPV 16 | E7 | 15-25 | LQPETTDLYCY |
| 185 | HPV 16 | E7 | 17-38 | PETTDLYCYEQLNDSSEEEDEI |
| 186 | HPV 16 | E7 | 18-25 | ETTDLYCY |
| 187 | HPV 16 | E7 | 18-26 | ETTDLYCYE |
| 188 | HPV 16 | E7 | 19-27 | TTDLYCYEQ |
| 189 | HPV 16 | E7 | 19-29 | TTDLYCYEQLN |
| 190 | HPV 16 | E7 | 21-40 | DLYCYEQLNDSSEEEDEIDG |
| 191 | HPV 16 | E7 | 21-42 | DLYCYEQLNDSSEEEDEIDGPA |
| 192 | HPV 16 | E7 | 35-50 | EDEIDGPAGQAEPDRA |
| 193 | HPV 16 | E7 | 42-52 | AGQAEPDRAHY |
| 194 | HPV 16 | E7 | 43-51 | GQAEPDRAH |
| 195 | HPV 16 | E7 | 43-52 | GQAEPDRAHY |
| 196 | HPV 16 | E7 | 43-53 | GQAEPDRAHYN |
| 197 | HPV 16 | E7 | 43-77 | GQAEPDRAHYNIVTFCCKCDSTLRLCVQST |
| | | | | HVDIR |
| 198 | HPV 16 | E7 | 44-52 | QAEPDRAHY |
| 199 | HPV 16 | E7 | 44-62 | QAEPDRAHYNIVTFCCKCD |
| 200 | HPV 16 | E7 | 46-55 | EPDRAHYNIV |
| 201 | HPV 16 | E7 | 47-57 | PDRAHYNIVTF |
| 202 | HPV 16 | E7 | 48-57 | DRAHYNIVTF |
| 203 | HPV 16 | E7 | 48-62 | DRAHYNIVTFCCKCD |
| 204 | HPV 16 | E7 | 49-57 | RAHYNIVTF |
| 205 | HPV 16 | E7 | 50-57 | AHYNIVTF |
| 206 | HPV 16 | E7 | 50-60 | AHYNIVTFCCK |
| 207 | HPV 16 | E7 | 50-62 | AHYNIVTFCCKCD |
| 208 | HPV 16 | E7 | 51-58 | HYNIVTFC |
| 209 | HPV 16 | E7 | 51-59 | HYNIVTFCC |
| 210 | HPV 16 | E7 | 51-72 | HYNIVTFCCKCDSTLRLCVQST |
| 211 | HPV 16 | E7 | 52-60 | YNIVTFCCK |
| 212 | HPV 16 | E7 | 53-60 | NIVTFCCK |
| 213 | HPV 16 | E7 | 56-65 | TFCCKCDSTL |
| 214 | HPV 16 | E7 | 62-75 | DSTLRLCVQSTHVD |
| 215 | HPV 16 | E7 | 64-78 | TLRLCVQSTHVDIRT |
| 216 | HPV 16 | E7 | 66-74 | RLCVQSTHV |
| 217 | HPV 16 | E7 | 67-76 | LCVQSTHVDI |
| 218 | HPV 16 | E7 | 67-98 | LCVQSTHVDIRTLEDLLMGTLGIVCPICSQKP |
| 219 | HPV 16 | E7 | 69-76 | VQSTHVDI |
| 220 | HPV 16 | E7 | 69-86 | VQSTHVDIRTLEDLLMGT |
| 221 | HPV 16 | E7 | 71-85 | STHVDIRTLEDLLMG |
| 222 | HPV 16 | E7 | 72-97 | THVDIRTLEDLLMGTLGIVCPICSQK |
| 223 | HPV 16 | E7 | 73-84 | HVDIRTLEDLLM |
| 224 | HPV 16 | E7 | 76-86 | IRTLEDLLMGT |
| 225 | HPV 16 | E7 | 77-86 | RTLEDLLMGT |
| 226 | HPV 16 | E7 | 77-87 | RTLEDLLMGTL |
| 227 | HPV 16 | E7 | 78-86 | TLEDLLMGT |
| 228 | HPV 16 | E7 | 79-87 | LEDLLMGTL |
| 229 | HPV 16 | E7 | 80-90 | EDLLMGTLGIV |
| 230 | HPV 16 | E7 | 81-90 | DLLMGTLGIV |
| 231 | HPV 16 | E7 | 81-91 | DLLMGTLGIVC |
| 232 | HPV 16 | E7 | 82-89 | LLMGTLGI |
| 233 | HPV 16 | E7 | 82-90 | LLMGTLGIV |
| 234 | HPV 16 | E7 | 82-91 | LLMGTLGIVC |
| 235 | HPV 16 | E7 | 82-92 | LLMGTLGIVCP |
| 236 | HPV 16 | E7 | 83-93 | LMGTLGIVCPI |
| 237 | HPV 16 | E7 | 84-93 | MGTLGIVCPI |
| 238 | HPV 16 | E7 | 85-93 | GTLGIVCPI |
| 239 | HPV 16 | E7 | 86-93 | TLGIVCPI |
| 240 | HPV 16 | E7 | 86-94 | TLGIVCPIC |
| 241 | HPV 16 | E7 | 87-97 | LGIVCPICSQK |
| 242 | HPV 16 | E7 | 88-97 | GIVCPICSQK |
| 243 | HPV 16 | E7 | 89-97 | IVCPICSQK |
| 244 | HPV 16 | E7 | 98 | VCPICSQKP |
| 245 | HPV18 | E6 | 13-21 | KLPDLCTEL |
| 246 | HPV18 | E6 | 36-43 | KTVLELTE |
| 247 | HPV18 | E6 | 40-48 | ELTEVFEFA |
| 248 | HPV18 | E6 | 43-57 | EVFEFAFKDLFVVYR |
| 249 | HPV18 | E6 | 51-72 | DLFVVYRDSIPHAACHKCIDFY |
| 250 | HPV18 | E6 | 71-92 | FYSRIRELRHYSDSVYGDTLEK |
| 251 | HPV18 | E6 | 125-135 | RRFHNIAGHYR |
| 252 | HPV18 | E7 | 1-32 | MHGPKATLQDIVLHLEPQNEIPVDLLCHEQL |
| | | | | S |
| 253 | HPV18 | E7 | 5-16 | KATLQDIVLHLE |
| 254 | HPV18 | E7 | 7-15 | TLQDIVLHL |
| 255 | HPV18 | E7 | 21-42 | IPVDLLCHEQLSDSEEENDEID |
| 256 | HPV18 | E7 | 86-94 | FQQLFLNTL |
| 257 | HPV18 | E7 | 88-97 | QLFLNTLSFV |
| 258 | HPV16 | E6V R17I | 9-17 | FQDPQERPI |
| 259 | HPV16 | E6V R17I | 9-19 | FQDPQERPIKL |
| 260 | HPV16 | E6V R17T | 15-25 | RPTKLPQLCTE |
| 261 | HPV16 | E6V Q21D | 18-28 | KLPDLCTELQT |
| 262 | HPV16 | E6V D32E | 25-33 | ELQTTIHEI |
| 263 | HPV16 | E6V D32E | 26-34 | LOTTIHEII |
| 264 | HPV16 | E6V D32E | 28-38 | TTIHEIILECV |
| 265 | HPV16 | E6V | 28-38 | TTIHEIRLECV |
| | | D32E, | | |
| | | I34R | | |
| 266 | HPV16 | E6V D32E | 29-38 | TIHEIILECV |
| 267 | HPV16 | E6V | 29-38 | TIHEIRLECV |
| | | D32E, | | |
| | | I34R | | |
| 268 | HPV16 | E6V D32E | 31-41 | HEIILECVYCK |
| 269 | HPV16 | E6V | 31-41 | HEIRLECVYCK |
| | | D32E, | | |
| | | I34R | | |
| 270 | HPV16 | E6V D32E | 32-41 | EIILECVYCK |
| 271 | HPV16 | E6V I34R | 34-41 | RLECVYCK |
| 272 | HPV16 | E6V A68G | 60-69 | VYRDGNPYGV |
| 273 | HPV16 | E6V H85Y | 79-89 | KISEYRYYCYS |
| 274 | HPV16 | E6V H85Y | 81-90 | SEYRYYCYSL |
| 275 | HPV16 | E6V | 81-90 | SEYRYYCYSV |
| | | H85Y, | | |
| 276 | HPV16 | E6V L90V | 81-91 | SEYRHYCYSVY |
| 277 | HPV16 | E6V L90V | 82-90 | EYRHYCYSV |
| 278 | HPV16 | E6V H85Y | 83-90 | YRYYCYSL |
| 279 | HPV16 | E6V L90V | 83-90 | YRHYCYSV |
| 280 | HPV16 | E6V | 83-90 | YRYYCYSV |
| | | H85Y, | | |
| | | L90V | | |
| 281 | HPV16 | E6V L90V | 83-91 | YRHYCYSVY |
| 282 | HPV16 | E6V L90V | 84-91 | RHYCYSVY |
| 283 | HPV16 | E6V H85Y | 84-93 | RYYCYSLYGT |
| 284 | HPV16 | E6V L90V | 84-94 | RHYCYSVYGTT |
| 285 | HPV16 | E6V L90V | 86-95 | YCYSVYGTTL |
| 286 | HPV16 | E6V L90V | 87-95 | CYSVYGTTL |
| 287 | HPV16 | E6V L90V | 87-96 | CYSVYGTTLE |
| 288 | HPV16 | E6V L90V | 88-95 | YSVYGTTL |
| 289 | HPV16 | E6V L90V | 89-99 | SVYGTTLEQQY |
| 290 | HPV16 | E6V L90V | 90-97 | VYGTTLEQ |
| 291 | HPV16 | E6V L90V | 90-99 | VYGTTLEQQY |
| 292 | HPV16 | E7V L28F | 21-28 | DLYCYEQF |
| 293 | HPV16 | E7V S63F | 56-63 | TFCCKCDF |
| 294 | HPV16 | E7V S63F | 56-65 | TFCCKCDFTL |
| 295 | HPV16 | E7V S63F | 56-66 | TFCCKCDFTLR |

In a further embodiment of the present invention the immunogenic fragment is any immunogenic fragment that is at least 75% identical to an immunogenic fragment derived from the E6 and/or E7 proteins of HPV16 and/or HPV18. Examples of variants of the immunogenic fragment derived from the E6 (E6V) and/or E7 (E7V) proteins of HPV16 and/or HPV18 are shown in Table 1 (SEQ ID NOs: 258-295).

Interaction of CD8+ and CD4+ T cells with antigen-presenting MHC class I and MHC class II, respectively, plays a pivotal role in the induction and maintenance of cytotoxic T cell responses, and are also important for direct anti-tumor immunity. The term “cytotoxic T cell response” refers to the specific recognition of pathogenic antigens, epitopes or immunogenic fragments and the induction of apoptosis in infected cells by cytotoxic T cells.

The composition according to the present invention is characterized in that the immunogenic fragment is able to bind to MHC class I and/or MHC class II.

In a preferred embodiment of the invention the immunogenic fragment is able to induce a cytotoxic T cell response.

Immunogenic fragments derived from the E6 and/or E7 proteins of HPV16 and/or HPV18, respectively, with the ability of binding to MHC class I and MHC class II, respectively, thereby inducing a cytotoxic T cell response, can be identified, for instance, by mass spectrometry (MS) of human HPV16+ and HPV18+ tumor cells, Enzyme Linked Immuno Spot (ELISpot) assay, in vitro cytotoxicity assay, anti-tumor activity measurements in MHC-humanized animal models, and bioinformatic approaches, such as MHC class I binding prediction tools (e.g., MHCflurry, MSIntrinsic, MixMHCPred, NetMHC, NetMHCpan, NetMHCcons).

In a preferred embodiment of the invention the immunogenic fragment comprises 3 to 35 amino acids. In particular, an immunogenic fragment comprises at least 3 amino acids, preferably at least 5, at least 6, at least 7, or at least 8 amino acids. In a preferred embodiment the immunogenic fragment does not exceed a length of 35 amino acids, preferably, it does not exceed a length of 25 amino acids, more preferably, it does not exceed a length of 20 amino acids. In a most preferred embodiment the immunogenic fragment has a length from 8 to 20 amino acids.

In an alternatively preferred embodiment of the invention the nanoparticles are loaded with pharmaceutically acceptable compounds comprising one or more HPV-derived immunogenic fragments and polyinosinic:polycytidylic acid (poly(I:C)) or any derivatives thereof.

In a preferred embodiment of the invention the derivatives of poly(I:C) are poly(I:C) LMW (Low Molecular Weight Poly(I:C)) with an average size from 0.2 kb to 1 kb, poly(I:C) HMW (High Molecular Weight Poly(I:C)) with an average size from 1.5 kb to 8 kb, polyl:polyC₁₂U.

According to the present invention, the nanoparticle(s) comprise silicon dioxide (SiO₂), optional in mixture with another material. The material thus can also be admixed with further components, where silicon dioxide typically has the highest proportion in a multicomponent system. The nanoparticles of the invention can comprise at least 80% of silicon dioxide, preferably at least 90%.

In a particularly preferred embodiment of the nanoparticles according to the invention, the material comprises silicon dioxide which is essentially pure, i.e. only comprises the impurities to be expected in the course of the preparation process. In a more preferred embodiment of the invention, the nanoparticle material consists of silicon dioxide.

Examples of other materials are metals, a metal chalcogenide, a magnetic material, a magnetic alloy, a semiconductor material, metal oxides, polymers, organosilanes, other ceramics or glass. The metal is selected from the group Au, Ag, Cu, Pt, Pd, Fe, Co, Gd, Ru, Rh and Zn, or any combination thereof.

In a further embodiment of the present invention the nanoparticles have a coating which comprises silicon dioxide. The core may comprise any other material such as metals, polymers or ferromagnetic metals such as Fe₂O₃ or Fe₃O₄. The core can even be devoid of silicon dioxide.

Silicon dioxide nanoparticles according to the invention have been described in, for example, WO 2010/006753 A2, which is expressly incorporated herein by reference.

The silicon dioxide nanoparticles can be prepared using, inter alia, the classical Stöber synthesis, in which monodisperse nanoscale silicon dioxide of defined size can be prepared by hydrolysis of tetraethoxysilane (TEOS) in aqueous-alcoholic-ammonia medium (J. Colloid Interface Sci. 1968, 26, 62).

The process of the preparation of silicon dioxide nanoparticles is described in detail in EP 0216 278 B1 and WO 2005/085135 A1, and consequently these documents are incorporated in their totality into the disclosure content of the present invention by way of reference. At least one amine is preferably used in the medium.

The silicon dioxide matrix of the nanoparticles according to the invention can be either porous or non-porous. The porosity is essentially dependent on the production process. In the synthesis in accordance with EP 0 216 278 B1, non-porous particles, in particular, are obtained.

According to the present invention the nanoparticles contain functional groups on their surface. These functional groups are capable to carry and/or stabilize both negative and positive charges. These charges may belong to pharmaceutically acceptable compounds such as (HPV)-derived immunogenic fragment and TLR agonists such as poly(I:C) and its derivatives.

Preferred TLR agonists are TLR3 agonists selected from the group consisting of poly(I:C) (dsRNA, TLR3 agonist, as well as RIG-I agonist), polyl:polyC₁₂U (dsRNA, TLR3 agonist, trade name Ampligen®, INN: Rintatolimod) and NAB2 (Nucleic acid band 2, dsRNA isolated from yeast and identified as an agonist of the pattern-recognition receptors TLR3 and MDA-5). More preferred is poly(I:C) LMW (Low Molecular Weight Poly(I:C) with an average size from 0.2 kb to 1 kb), poly(I:C) HMW (High Molecular Weight Poly(I:C) with an average size from 1.5 kb to 8 kb), polyl:polyC₁₂U or poly-ICLC. Most preferred is poly(I:C) LMW.

Functional groups which are capable to carry and/or stabilize both negative and positive charges are for example —SH, —COOH, —NH₂, -guanidino-group (—NHC(═NH))NH₂), —PO₃H₂, —PO₂CH₃H, —SO₃H, —OH, —NR₃⁺X⁻. Preferred functional groups are —COOH, -guanidino-group (—NHC(═NH))NH₂) and —NH₂. The functional groups may also be present in their salt form.

Compositions according to the invention comprise nanoparticles which have a surface loading density up to 0.5, preferably between 0.01 and 0.5, more preferred between 0.03 and 0.4, most preferred between 0.05 to 0.3, in relation to the total number of the pharmaceutically acceptable compounds with regard to the surface of the nanoparticle in nm² [molecules/nm²].

This surface loading density is calculated by assuming a perfect sphere and by the molar loading per particle. For example: a spherical particle with a diameter of 25 nm has a surface of 1964 nm² (A=π*d²) and a weight of 1.64E-8 ng (with an assumed density of 2000 kg/m³ for amorphous silica). The loading of such a particle with 5% by weight with the peptide KKKV-Cit-YMLDLQPET (M=1,910.31 g/mol) equals to 4.283E-22 mol. This value multiplied by the Avogadro constant results in 258 single molecules of KKKV-Cit-YMLDLQPET attached to one particle with 1964 nm². This corresponds to a surface loading density of 0.13 molecules/nm².

The compositions according to the invention are formulated to have a pH between 6.0 and 8.0, preferably between 6.5 and 7.8 and more preferably between 6.8 and 7.5, even more preferred between 7.2 and 7.4. The pH of a composition can be maintained by the use of a buffer such as acetate, citrate, phosphate, succinate, TRIS (tris(hydroxymethyl)aminomethane) or histidine, typically employed in the range from about 1 mM to 50 mM. The pH of compositions can otherwise be adjusted by using physiologically acceptable acids or bases.

In one embodiment of the present invention the functional groups are —PO₃H groups. Phosphorylated nanoparticles according to the invention could be for example prepared by reaction of (diethylphosphatoethyl)triethoxysilane with silica nanoparticles under addition of ammonia.

In a preferred embodiment of the present invention the functional groups are connected to the nanoparticle via a linker L. The linker can be connected to the nanoparticles e.g. by way of a covalent or adsorptive bond.

In a preferred embodiment of the present invention the linker compound L comprises at least one carboxyl (—COOH) or carboxylate (—COO⁻) group as functional group.

In a more preferred embodiment such linker further comprises at least one guanidino-group (—NHC(═NH)NH₂ or —NHC(═NH₂⁺)NH₂) or amino-group (—NH₂ or —NH₃⁺) as functional group.

In a most preferred embodiment of the present invention the linker compound L contains at least a structural unit of the general formula (1)

*-(—O)₃Si—(CH₂)ₙ—CH(COOX)—(CH₂)ₚ—C(O)—NH—CH(COOX)—(CH₂)_{q}—Y (I)

wherein
X is independently from each other H or a negative charge,
Y is independently from each other —NHC(═NH)NH₂, —NHC(═NH₂+)NH₂, —NH₂ or —NH₃⁺,
n, p and q are independently from each other 0 or a number from 1 to 25; and
*- is the connection point to the nanoparticle.

In a preferred embodiment the linker compounds L contain at least a structural unit of formula (1) wherein n is 3, p is 1 and q is 4 and Y is independently from each other a —NH₂ or —NH₃⁺ group.

In a particularly preferred embodiment the linker compounds L contain at least a structural unit of formula (1) wherein n is 3, p is 1 and q is 3 and Y is independently from each other a —NHC(═NH)NH₂ or —NHC(═NH₂+)NH₂ group.

Another aspect of the present invention is to provide a process of preparation of nanoparticles according to the invention wherein
in a first step (i) hydrolytic polycondensation of tetraalkoxysilanes and/or organotrialkoxysilanes in a medium which comprises water, at least one solubilizer and at least one amine or ammonia take place, where firstly a sol of primary particles is produced, and the resultant nanoparticles are subsequently brought to the desired particle size in a range from 5 to 150 nm in such a way that further nucleation is limited by continuous metering-in of corresponding silane in a controlled manner corresponding to the extent of reaction, and
in a second step (ii) the nanoparticles from step (i) are reacted with [(3-triethoxysilyl)propyl]succinic anhydride, which in a simultaneous reaction forms an amide with L-arginine or L-lysine.

It is preferred to use in step (ii) of the process L-arginine.

In an alternative embodiment the linker compound L could be also obtained by the reaction of L-arginine with N-(3-triethoxysilylpropyl)maleimide. The reaction is carried out preferably at pH values above 8.

In one embodiment of the present invention it also possible to produce the linker compound L by reaction of [(3-triethoxysilyl)propyl]succinic anhydride with agmatine, histamine, cadaverine or spermidine.

A further embodiment of the present invention are nanoparticles comprising a silicon dioxide based surface with a linker compound L covalently or adsorptive bonded to it, wherein the Linker L contains at least one guanidino-group (—NHC(═NH)NH₂ or —NHC(═NH₂+)NH₂) or amino-group (—NH₂ or —NH₃⁺) as a functional group. In a preferred embodiment the Linker L contains at least one guanidino-group (—NHC(═NH)NH₂ or —NHC(═NH₂+)NH₂) or amino-group (—NH₂ or —NH₃⁺) and at least one carboxyl (—COOH) or carboxylate (—COO—) group as a functional group. In a more preferred embodiment the Linker L contains at least one guanidino-group (—NHC(═NH)NH₂ or —NHC(═NH₂+)NH₂) and at least one carboxyl (—COOH) or carboxylate (—COO—) group as a functional group.

One embodiment of the nanoparticles according to the invention is illustrated in FIG. 3.

FIG. 3 shows the schematic drawing of the cross section of a nanoparticle according to the invention. A represents the surface functionalization with the linker compound L, B represents the amorphous SiO₂ shell and C represents the core material, which could be void, water or any other material as well as amorphous SiO₂. The diameter d1 is between 0 and 149 nm and d2 is between 10 and 150 nm.

In one embodiment of the invention the pharmaceutically acceptable compound is conjugated to the nanoparticle by adsorptive or covalent attachment. The adsorptive attachment is preferred.

A further object of the present invention are nanoparticles having silicon dioxide and functional groups on the surface and a particle size below 150 nm, comprising a linker L which is covalently or adsorptive bonded to it, wherein the Linker L comprises at least one guanidino-group (—NHC(═NH)NH₂ or —NHC(═NH₂+)NH₂) or amino-group (—NH₂ or —NH₃⁺) group as a functional group.

A main advantage of the adsorptive binding of the pharmaceutically acceptable compound is that, in contrast to most covalent conjugations, no by-products are formed or remain in the “reaction” mixture. In case of a covalent attachment it could become necessary to chemically modify the molecule which should be attached by adding reactive functional groups to the molecule. This is a fundamental intervention in the structure of the peptide/antigen and constitutes a complex chemical modification in case of RNA- or DNA-based antigens or adjuvants. In addition, a covalent linkage of a known compound that has already been approved by the drug authorities creates a new, independent substance (New Chemical Entity, NCE) which, for regulatory reasons, requires a new approval.

In a preferred embodiment the linker compound L according to formula (1) is able to carry and/or stabilize by way of adsorption pharmaceutically acceptable compounds which have positive or negative charges.

In a particularly preferred embodiment the linker compound L according to formula (1) is able to carry and/or stabilize by way of adsorption pharmaceutically acceptable compounds which have negative charges.

In a more preferred embodiment the linker compound L according to formula (1) is able to carry and/or stabilize by way of adsorption pharmaceutically acceptable compounds which have phosphate or phosphonate groups.

It was surprisingly found that the electrostatic interaction between the arginine group of the linker compound L and the negative charge of the phosphate group of the pharmaceutically acceptable compound possesses a ‘covalent-like’ stability.

The term “attachment” here relates to any type of interaction between the surface functionality and the antigen, in particular covalent and non-covalent bonds, such as, for example, hydrophobic/hydrophilic interactions, van der Waals forces, ionic bonding, hydrogen bonds, ligand-receptor interactions, base pairing of nucleotides or interactions between epitope and antibody binding site.

In case where the pharmaceutically acceptable compound is hydrophobic, for example a hydrophobic peptide, it is advantageous to increase the hydrophilicity thereof. In case of a protein or peptide this is possible e.g. by an N- or C-terminal extension with polar amino acids. In a preferred embodiment of the invention the protein or peptide is extended with one or more polar amino acids selected from the group comprising aspartic acid, glutamic acid, histidine, lysine, arginine, serine, threonine or tyrosine, preferably lysine, arginine, glutamic acid and aspartic acid more preferred lysine.

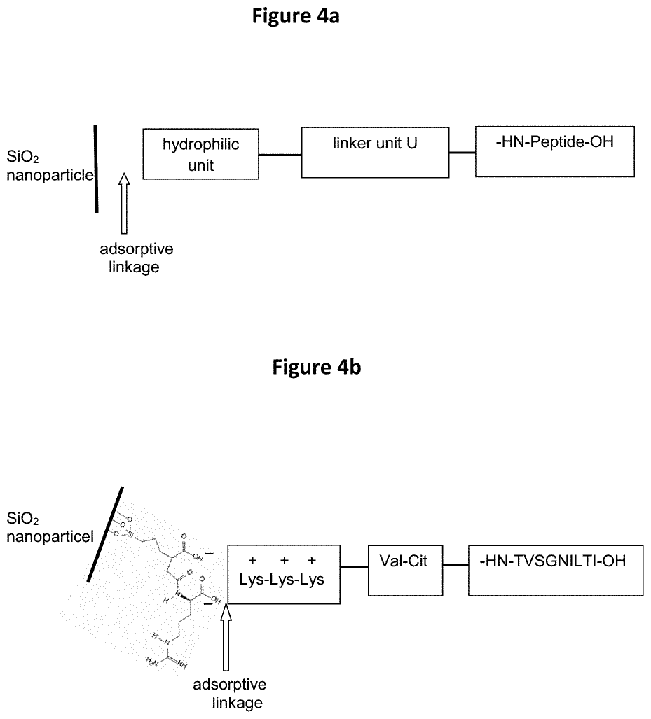

The present invention is therefore particularly directed to a tripartite bioconjugate which comprises an N- or C-terminal extension with polar amino acids for enhancing the solubility and which is connected to the linker compound L by adsorptive linkage, a linker unit U and an antigen/epitope which is illustrated in FIG. 4a.

A linker unit U according to the invention is for example used in antibody-drug conjugates (ADCs) which contain various types of linkers. There are three main types of chemically cleavable linkers: acid cleavable, reducible disulfides and those cleavable by exogenous stimuli.

Examples of ADCs are Gemtuzumab ozogamicin (Mylotarg® by Pfizer, linker is 4-(4-acetylphenoxy)butanoic acid), Inotuzumab ozogamicin (Besponsa® by Pfizer, linker is condensation product of 4-(4′-acetylphenoxy)-butanoic acid (AcBut) and 3-methyl-3-mercaptobutane hydrazide (known as dimethylhydrazide), Trastuzumab emtansin (Kadcyla® by Roche, linker is 4-[N-Maleimidomethyl]cyclohexan-1-carboxylate) or Brentuximab vedotin (also known as SGN-035; Adcetris® by Seattle Genetics Inc., linker is the dipeptide valine-citrulline).

In a preferred embodiment of the present invention the peptides comprise as linker unit U comprising an N-terminal extension with an enzymatic cathepsin B-cleavable linker (catB-cleav-linker) to ensure the release of the native unmodified antigen for MHC class I or MHC class II. The enzymatic catB-cleav-linker comprises one of the cathepsin B sensitive dipeptides Val-Cit, Phe-Cit, Leu-Cit, Ile-Cit, Trp-Cit, Phe-Lys, Ala-Lys or Val-Lys, preferred dipeptides are Val-Cit or Trp-Cit, more preferred is Val-Cit.

In one embodiment the compositions according to the invention comprise phosphorylated nanoparticles and one or more peptides which are conjugated to the nanoparticles by adsorptive attachment, wherein the peptide comprises Val-Cit or Trp-Cit as cathepsin B sensitive dipeptide.

FIG. 4b shows a preferred tripartite bioconjugate according to the invention. The peptide used in FIG. 4b is an extreme hydrophobic epitope derived from human NY-ESO-1 (SEQ ID NO: 297). For the N-terminal extension the enzymatic (cathepsin B) cleavable peptidic linker Val-Cit was used to ensure the release of the native unmodified antigen for MHC class I or MHC class II. For enhancing the solubility and the electrostatic attraction to the nanoparticle the extension part “Lys-Lys-Lys” was used. Also possible is the extension for example with Lys-Lys-Lys-Asp or Arg₆.

In a preferred embodiment of the present invention the HPV16 E7₈₂₋₉₀ epitope LLMGTLGIV (SEQ ID NO: 233) is enlarged on the N-terminal site with the enzymatic cleavable linker Val-Cit and the cationic solubilizing sequence Lys-Lys-Lys, leading to KKKV-Cit-LLMGTLGIV.

In another preferred embodiment of the present invention the HPV16 E7₁₁₋₁₉ epitope YMLDLQPET (SEQ ID NO: 174) is enlarged on the N-terminal site with the enzymatic cleavable linker Trp-Cit and the cationic solubilizing sequence Lys-Lys-Lys leading to KKKW-Cit-YMLDLQPET.

A further embodiment is a composition according to the invention comprising SiO₂-nanoparticles having linker compounds L on the surface, wherein the linker compound L comprises at least one guanidino-group (—NHC(═NH)NH₂ or —NHC(═NH₂+)NH₂) and at least one carboxyl (—COOH) or carboxylate (—COO—) group as functional groups and one or more peptides or antigens which are conjugated to the linker compound L by adsorptive attachment, wherein the peptide or antigen comprises a linker unit U and a hydrophilic elongation with one or more amino acids.

A preferred embodiment is a composition according to the invention comprising SiO₂-nanoparticles having linker compounds L on the surface, wherein the linker compound L comprises at least one guanidino-group (—NHC(═NH)NH₂ or —NHC(═NH₂+)NH₂) and at least one carboxyl (—COOH) or carboxylate (—COO—) group as functional groups and one or more peptides or antigens which are conjugated to the linker compound L by adsorptive attachment, wherein the peptide or antigen comprises a cathepsin B sensitive dipeptide and a hydrophilic elongation with Lys-Lys-Lys.

A more preferred embodiment is a composition according to the invention comprising SiO₂-nanoparticles having linker compounds L on the surface, wherein the linker compound L comprises at least one guanidino-group (—NHC(═NH)NH₂ or —NHC(═NH₂+)NH₂) and at least one carboxyl (—COOH) or carboxylate (—COO—) group as functional groups and one or more peptides or antigens which are conjugated to the linker compound L by adsorptive attachment, wherein the peptide or antigen comprises Val-Cit or Trp-Cit as cathepsin B sensitive dipeptide and a hydrophilic elongation with Lys-Lys-Lys.

Another embodiment is a composition according to the invention comprising SiO₂-nanoparticles having linker compounds L on the surface, wherein the linker compound L comprises at least one guanidino-group —HC(═NH)NH₂ or —NHC(═NH₂⁺)NH₂) and at least one carboxyl (—COOH) or carboxylate (—COO—) group as functional groups and the model peptide SIINFEKL which is conjugated to the linker compound L by adsorptive attachment, wherein the peptide comprises Val-Cit or Trp-Cit as cathepsin B sensitive dipeptide and a hydrophilic elongation with Lys-Lys-Lys.

However, this hydrophilic modification of the peptide could lead to a stronger binding to MHC class I thereby displacing the target epitope and could provoke an immune response against an epitope not present on the tumor cells. To avoid this effect, there could be the need of a spacer between the hydrophilizing sub-molecule and the epitope.

An optional embodiment of the present invention is the use of so-called self-immolative spacers (PABC, p-aminobenzylcarbamate). Examples of such self-immolative spacers are compounds which comprise the structural unit of 4-aminobenzyl alcohol, 2-aminobenzyl alcohol or 4-hydroxybenzyl alcohol, 2-hydroxybenzyl alcohol (EP-A 0 648 503, WO 2007/031734 A1, WO 2015/162291 A1, U.S. Pat. No. 6,180,095 B1, U.S. Pat. No. 6,214,345 B1).

A self-immolative spacer is defined as a molecular section which is chemically linked to at least two further molecular sections such that when one of the bonds to the molecular sections is released, the remaining bonds are split and the previously attached molecules are released.

If a self-immolative spacer is used, it is preferred that the spacer is placed between enzymatic catB-cleav-linker and the peptide. As an example the following bioconjugate could be produced: (Arg₆)-(Val-Cit)-(PABC)-(SIINFEKL), wherein SIINFEKL (SEQ ID NO: 296) is a model antigen.

It was surprisingly found that the enzymatic catB-cleav-linker systems can be used without the need of a self-immolative spacer.

The epitopes used for therapeutic vaccination typically consist of 8 to 11 amino acids for MHC class I and a maximum of 25 amino acids for MHCclass II. For those epitopes there is no need for using a self-immolative spacer, because of the small size of these epitopes in comparison to much larger drugs, inhibiting an enzymatic cleavage A more preferred embodiment is a composition according to the invention comprising SiO₂-nanoparticles having linker compounds L on the surface, wherein the linker compound L comprises at least one guanidino-group (—NHC(═NH)NH₂ or —NHC(═NH₂+)NH₂) and at least one carboxyl (—COOH) or carboxylate (—COO—) group as functional groups and poly(I:C) or any derivatives thereof and one or more immunogenic fragments which are conjugated to the linker compound L by adsorptive attachment, wherein the an immunogenic fragment comprises a linker unit U and a hydrophilic elongation with one or more amino acids.

In specific embodiments, the immunogenic fragment can be selected from the group consisting of the following:
(a) peptides suitable to induce an immune response against infectious diseases;
(b) peptides suitable to induce an immune response against cancer cells.

In some embodiments, the immunogenic fragment is one that is useful for the prevention of infectious disease. Such treatment will be useful to treat a wide variety of infectious diseases affecting a wide range of hosts, preferably human, but including cow, sheep, pig, dog, cat, and other mammalian species and non-mammalian species.

Examples of cancers include, but are not limited to cervical cancer, anogential cancer, head and neck cancer or cytological abnormalities such as atypical squamous cells of undetermined significance (ASCUS) or any cancer caused by one or more HPV types.

Another example of a disease which is caused by a HPV infection is cervical intraepithelial neoplasia grade 1 (CIN1), CIN2, CIN3. Cervical intraepithelial neoplasia (CIN), also known as cervical dysplasia, is the abnormal growth of cells on the surface of the cervix that could potentially lead to cervical cancer. More specifically, CIN refers to the potentially precancerous transformation of cells of the cervix.

One example of an infectious disease is a persistent infection with a high-risk HPV type.

HPV-derived immunogenic fragments are commonly used for immunoprophylaxis of HPV-related conditions, for instance, as vaccines or as immunostimulant.

The term “vaccine” or “immunostimulant” refers to a composition that comprises an immunogenic fragment capable of provoking an immune response in an individual, such as a human, wherein the composition optionally contains an adjuvant. A vaccine for HPV suitably elicits a protective immune response against incident infection, or persistent infection, or cytological abnormalities such as ASCUS, CIN1, CIN2, CIN3, or cancer caused by one or more HPV types.

Another object of the present invention is the composition comprising nanoparticles which are loaded with pharmaceutically acceptable compounds comprising one or more HPV-derived immunogenic fragments or a variant thereof according to the present invention for use as vaccines or as immunostimulant.

In one embodiment the compositions according to the invention are used as vaccines for personalized cancer treatment.

The compositions according to the invention are used as vaccines for the prevention of HPV infection or the treatment of HPV-positive humans or the treatment of HPV-positive tumors.

Also object of the present invention is a lyophilisate comprising nanoparticles according to the invention. The lyophilisate may comprise additives for example polymers (e.g. polyethylene glycol, polyvinyl pyrrolidone, hydroxyethyl starch, dextran and ficoll) and sugars, (e.g. trehalose, lactose, sucrose, glucose, galactose, maltose, mannose and fructose), polyhydroxy alcohols (e.g. mannitol, sorbitol and inositol), amino acids (e.g. glycine, alanine, proline and lysine) and methylamines (e.g. trimethylamine-N-oxide, betaine and sarcosine). Lyophilisates according to the invention could be resuspended with sterile water before vaccination.

A further object of the present invention is to provide a vaccine comprising compositions according to the invention.

Compositions according to the present invention are preferably stable dispersions.

The nanoparticles can be in dispersed form in any desired solvent, so long as the nanoparticles are neither chemically attacked nor physically modified by the solvent, and vice versa, so that the resultant nano-dispersion is stable, in particular pharmaceutically and physically stable. The dispersion is specifically characterized in that the nanoparticles are in monodisperse and non-aggregated form and have no tendency towards sedimentation, which results in sterile filterability.

A pharmaceutical composition according to the present invention is any composition which can be employed in the prophylaxis, therapy, control or post-treatment of patients who exhibit, at least temporarily, a pathogenic modification of the overall condition or the condition of individual parts of the patient organism, in particular as a consequence of infectious diseases, tumors or cancer. Thus, in particular, it is possible for the pharmaceutical composition in the sense of the invention to be a vaccine and/or an immunotherapeutic agent.

The compositions according to the invention may be formulated as pharmaceutical compositions that may be in the forms of solid or liquid compositions. Physiological saline solution or glycerol or glycols such as propylene glycol or polyethylene glycol may be included.

The compositions according to the present invention optionally may comprise other active ingredients or may comprise one or more of a pharmaceutically acceptable excipient, carrier, buffer, stabilizer, isotonic agent, preservative or anti-oxidant or other materials well known to those skilled in the art. Such materials should be non-toxic and should not interfere with the efficacy of the pharmaceutically acceptable compound. The precise nature of the carrier or other material may depend on the route of administration, e.g. orally or parenterally.

For intravenous, cutaneous or subcutaneous injection, or injection at the site of affliction, the composition according to the present invention will be in the form of a parenterally acceptable aqueous solution which is pyrogen-free and has suitable pH, isotonicity and stability.

Preservatives are generally included in compositions according to the invention to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalkonium chloride, 1-thioglycerol and benzethonium chloride.

The nanoparticle-containing compositions of the invention may be administered to patients by any number of different routes, including enteral or parenteral routes. Parenteral administration of the pharmaceutical composition is preferred. Parenteral administration includes administration by the following routes: cutaneous or subcutaneous, nasal, vaginal, rectal, intramuscular, intraocular, transepithelial, intraperitoneal, intracardiac, intraosseous, intradermal, intrathecal, intraperitoneal, transdermal, transmucosal, and inhalational and topical (including dermal, ocular, rectal, nasal, vaginal, inhalation and aerosol), and rectal systemic routes. In a preferred embodiment, the pharmaceutical composition is for local (e.g., mucosa, skin) applications.

Administration be performed e.g. by injection, or ballistically using a delivery gun to accelerate their transdermal passage through the outer layer of the epidermis. The nanoparticles can then be taken up, e.g. by dendritic cells, which mature as they migrate through the lymphatic system, resulting in modulation of the immune response and vaccination against the epitopic peptide and/or the antigen from which the epitopic peptide was derived or of which it forms a part. The nanoparticles may also be delivered in aerosols. This is made possible by the small size of the nanoparticles.

Particularly preferred is the injection an intradermal, subcutaneous, intramuscular injection. The administration can be carried out, for example, with the aid of so-called vaccination guns or by means of syringes. Mucosal administration, in particular mucosal vaccination, can be beneficial for cases were pathogens enter the body via the mucosal route. Mucosal delivery routes primarily include the oral and rectal route. Sometimes a pretreatment of the mucosa is necessary. It is also possible to prepare the substance as an aerosol, which is inhaled by the organism, preferably a human patient and taken up by the nasal and or bronchial mucosa. Other possible forms of mucosal administration are vaginal and rectal suppositories. These forms of administration are cost-effective (no consumables like syringes and needles), safe (very low risk of infection and operating errors), easy to apply and comprised with a high patients compliance (no needle fear). It also addresses the mucosal immune system (MALT) directly.

In one embodiment of the present invention the vaccines comprising the compositions according to the invention are used for a mucosal administration.

The exceptionally small size of the nanoparticles of the present invention is a great advantage for delivery to cells and tissues, as they can be taken up by cells even when linked to targeting or therapeutic molecules. Thus, the nanoparticles may be internalized by APCs, the immunogenic fragments processed and presented via MHC class I and MHC class II.

### PARTICULARLY PREFERRED EMBODIMENTS OF THE INVENTION

1. Composition comprising nanoparticles which are loaded with pharmaceutically acceptable compounds comprising one or more human papilloma virus (HPV)-derived immunogenic fragments or a variant thereof, having silicon dioxide and functional groups on the surface, wherein
   the functional groups are capable to carry and/or stabilize both negative and positive charges of the pharmaceutically acceptable compounds,
   the zeta potential of the composition has a value of at least ±15 mV,
   the nanoparticles have a particle size below 150 nm.
2. Composition according to embodiment 1 characterized in that the immunogenic fragment is derived from a high-risk HPV genotype, preferably selected from HPV types 16, 18, 31, 33, 35, 39, 45, 51, 52, 56, 58, 59, 66, 68, 73, and 82.
3. Composition according to embodiment 1 or 2 characterized in that the immunogenic fragment is derived from the E6 and/or E7 proteins of HPV16 and/or HPV18, preferably selected from SEQ ID NOs: 5-257.
4. Composition according to any of embodiments 1 to 3 characterized in that the immunogenic fragment is at least 75% identical to an immunogenic fragment derived from the E6 and/or E7 proteins of HPV16 and/or HPV18, preferably selected from SEQ ID NOs: 258-295.
5. Composition according to any of embodiments 1 to 4 characterized in that the immunogenic fragment is able to bind to major histocompatibility complex (MHC) class I and/or MHC class II.
6. Composition according to any of embodiments 1 to 5 characterized in that the immunogenic fragment is able to induce a cytotoxic T cell response.
7. Composition according to any of embodiments 1 to 6 characterized in that the immunogenic fragment has a length of 3 to 35 amino acids, more preferably of 5 to 25 amino acids, more preferably of 6 to 25 amino acids, more preferably of 7 to 25 amino acids, more preferably of 8 to 25 amino acids, most preferably of 8 to 20 amino acids.
8. Composition according to any of embodiments 1 to 7, wherein the nanoparticles are loaded with pharmaceutically acceptable compounds comprising one or more human papilloma virus (HPV)-derived immunogenic fragments and polyinosinic:polycytidylic acid (poly(I:C)) or any derivatives thereof.
9. Composition according to embodiment 8, wherein derivatives of poly(I:C) are poly(I:C) LMW (Low Molecular Weight Poly(I:C)) with an average size from 0.2 kb to 1 kb, poly(I:C) HMW (High Molecular Weight Poly(I:C)) with an average size from 1.5 kb to 8 kb, polyl:polyC₁₂U.
10. Composition according to embodiments 1 to 9, wherein the nanoparticles have a surface loading density up to 0.5, preferably between 0.01 and 0.5, more preferred between 0.03 and 0.4, most preferred between 0.05 to 0.3, in relation to the total number of the pharmaceutically acceptable compounds with regard to the surface of the nanoparticle in nm² [molecules/nm²].
11. Composition according to embodiments 1 to 10, wherein the Polydispersity Index (PDI) of the composition is between 0 and 0.32, preferably between 0.1 and 0.3, more preferably between 0.1 and 0.2, most preferred less than 0.1.
12. Composition according to any of embodiments 1 to 11, wherein the pH value of the composition is between 6.0 and 8.0.
13. Composition according to any of the preceding embodiments, wherein the zeta potential of the composition has a value of at least ±30 mV.
14. Composition according to any of the preceding embodiments, wherein the Z-average diameter of the nanoparticles is in the range of ≥5 and <150 nm, preferably ≥15 and ≤60 nm, more preferably ≥20 and ≤50 nm and still more preferably between 20 and 30 nm.
15. Composition according to any of the preceding embodiments, wherein the net charge of the loaded nanoparticles in total is different to zero.
16. Composition according to any of the preceding embodiments, wherein the functional groups are connected to a linker L which is linked to the surface of the nanoparticles by way of a covalent or adsorptive bond.
17. Composition according to embodiment 16, wherein the linker compound L comprises at least one carboxyl (—COOH) or carboxylate (—COO—) group as functional group.
18. Composition according to embodiment 16 or 17, wherein the linker compound L comprises at least one guanidino-group (—NHC(═NH)NH₂ or —NHC(═NH₂+)NH₂) or amino-group (—NH₂ or —NH₃⁺) group as a functional group.
19. Composition according to any of embodiments 16 to 18, wherein the linker L comprises at least one further functional group L′, which is selected from the group —SH, —COOH, —NH₂, -guanidino-group (—NHC(═NH))NH₂), —PO₃H₂, —PO₂CH₃H, —SO₃H, —OH, —NR₃⁺X⁻.
20. Composition according to any of embodiments 10 to 13, wherein the linker compound L contains at least a structural unit of formula (1)

*-(—O)3Si—(CH2)n-CH(COOX)—(CH2)p-C(O)—NH—CH(COOX)—(CH2)q-Y (I)

wherein
X is independently from each other H or a negative charge,
Y is independently from each other —NHC(═NH)NH₂, —NHC(═NH₂+)NH₂, —NH₂ or —NH₃+,
n, p and q are independently from each other 0 or a number from 1 to 25; and
*- is the connection point to the nanoparticle.
21. Composition according to any of the preceding embodiments, wherein the pharmaceutically acceptable compound is conjugated to the nanoparticle by adsorptive attachment.
22. Composition according to any of the preceding embodiments, wherein the immunogenic fragment is N- or C-terminally extended with polar amino acids.
23. Composition according to embodiment 22, wherein the polar amino acids are selected from the group aspartic acid, glutamic acid, histidine, lysine, arginine, serine, threonine, tyrosine.
24. Composition according to embodiment 22 or 23, wherein the peptides comprise an N-terminal extension with an enzymatic cathepsin B-cleavable linker.
25. Composition according to embodiment 24, wherein the enzymatic cleavable linker comprises one of the cathepsin B sensitive dipeptides Val-Cit, Phe-Cit, Leu-Cit, Ile-Cit, Trp-Cit, Phe-Lys, Ala-Lys or Val-Lys.
26. Composition according to embodiment 24 or 25, wherein a spacer is placed between enzymatic cleavable linker and peptide.
27. Composition according to embodiment 26, wherein the spacer is a self-immolative spacer.
28. Composition according to embodiment 26 or 27, wherein the spacer comprises the structural unit of 4-aminobenzyl alcohol, 2-aminobenzyl alcohol or 4-hydroxybenzyl alcohol, 2-hydroxybenzyl alcohol.
29. Composition according to any of the preceding embodiments, wherein the nanoparticles comprise at least silicon dioxide (SiO₂), optional in mixture with another material.
30. Composition according to any of the preceding embodiments, wherein the nanoparticles consist of SiO₂. 31. Composition according to any of the preceding embodiments for use as vaccine or as immunostimulant.
32. Composition according to any of the preceding embodiments for personalized cancer treatment.
33. Composition according to any of the preceding embodiments for use as vaccine for the prevention of HPV infection or the treatment of HPV positive humans or the treatment of HPV positive tumors.
34. Lyophilisate comprising compositions according to any of the preceding embodiments.
35. Vaccine comprising compositions according to any of the preceding embodiments.
36. Composition according to any of the preceding embodiments, wherein the compositions are stable dispersions.
37. Composition according to any of the preceding embodiments, wherein the pH value of the composition is between 7.2 and 7.4.
38. Composition according to any of the preceding embodiments, wherein the composition is isotonic.
39. Composition according to any of the preceding embodiments, wherein the composition contains further pharmaceutically acceptable additives.

### Examples

### Example 1

Preparation of Monodisperse Silicon Dioxide Nanoparticles with 25 nm Diameter

500 mL ethanol absolute were taken from a 500 mL graduated measuring cylinder into a 1000 mL glass bottle with screw cap. 358 mL sterile DI water was added and the mixture was well shaken.

69.6 mL tetraethylorthosilicate (TEOS) were added to the bottle. The bottle was tightly closed and well shaken. After 1 hour waiting time the clear colorless mixture got room temperature (22° C.). Then 9.5 mL ammonia water (25%) was added quickly.

The bottle was shaken by hand very well for 10 seconds and stored at room temperature.

After 24 hours at room temperature additional 34.8 mL TEOS were added to the mixture (seed-growth process), in order to get a more narrow size distribution. After additional 24 hours at room temperature again 34.8 mL were added to the mixture (seed-growth process continued). After 24 hours at room temperature 50 μL of the reaction mixture were placed in a one-way PMMA cuvette (10 mm width) and diluted with 1.5 mL DI water (0.2 μm filtered). The cuvette was placed in a ZetaSizer Nano for particle size measurement (ZetaSizer Nano ZS, Malvern Instruments, UK).

Applied Parameters:
Measurement Angle: 1730 Backscatter (NIBS default)
Measurement Cell: DTS1070 (used for particle size and zeta potential)
Refractive Index SiO2: 1.460
Absorption: 0.010
Dispersant: water
Refractive Index: 1.330
Viscosity: 0.8872 cP (═sample viscosity)
Temperature: 25° C.
>10 measurement runs for particle size

Results:
Z-average (volume based): 23.86 nm
PDI: 0.108
Intensity Peak: 26.77 nm
pH: 11.0 (freshly calibrated combination electrode)

The nanoparticle suspension was transferred into a 2 liter round bottom flask and the ethanol as well as the ammonia gas was removed by a rotary evaporator with heated water bath. 400 mL sterile DI water was added in portions. The volume was reduced down to 198.15 g.

The solid content (pure nano dispersed silicon dioxide) of the suspension was determined in triplicate via vaporization of 250 μL and weigh out the residue.

Result:
Solid content: 163.3 mg/mL
pH: 8.0 (freshly calibrated combination electrode)

The distillation residue was diluted with 264.5 mL sterile DI water, in order to get a solid content of 70.0 mg/mL.

### Example 2

Functionalization of the Nanoparticles with L-Arginine to Get Arginylated Silica Nanoparticles (SiO₂-Arg)

462.65 g of the nanoparticle suspension obtained in Example 1 were placed into a 500 mL glass bottle. A magnetic stir was added and the bottle was placed onto a magnetic stirrer. 17.35 g L(+)-Arginine (CAS No. 74-79-3, PanReac AppliChem, Ph. Eur., USP. product code A1345.0500) were added to the nanoparticle suspension. When the arginine was completely dissolved a pH value of 10.8 was obtained, then 5.34 mL 3-(Triethoxysilyl)propylsuccinic anhydride (CAS No. 93642-68-3, Gelest Corp. product code: SIT8192.6) were slowly added at room temperature. The reaction mixture got very turbid but cleared up within about 30 minutes. During this time two reactions took place simultaneously:

The ethoxy groups of the silane got hydrolyzed, due to the high pH value. The created silanol groups precipitated onto the silica nanoparticles building a surface coating on top of the nanoparticles. In parallel the amino group of the excess arginine reacts with the succininc anhydride group, forming an amide bond.

The pH value dropped to 9.8, due to the consumption of arginine and mainly due to the formation of a carboxylic group, by opening the cyclic anhydride.

After 24 hours of stirring the suspension was transferred into 20 falcons tubes with a 100 kDa membrane (Pall Corp. Macrosep® Advance, product code MAP100038). The nanoparticle suspension was centrifuged for 10 minutes at 4,000 rpm (=2,737 g at the used centrifuge) to a volume at least 5 less than the starting volume. After centrifugation the volume in the falcon tubes was restored with sterile DI water and the centrifugation was repeated 5 times. The centrifugation step is necessary to remove excess arginine and possible unbound reaction products.

After centrifugation the solid content of the collected supernatants was determined in triplicate. The method was the same as in Example 1.

Result:
Yield: 178.3 g
Solid content: 97.16 mg/mL The nanoparticle suspension was diluted with 168.17 mL sterile DI water to get a final concentration of 50.0 mg/mL.

### Example 3

Synthesis of Phosphorylated Silica Nanoparticles with 25 nm Diameter

200 mL ethanol absolute were taken from a 250 mL graduated measuring cylinder into a 500 mL pressure-resistant glass bottle with screw cap 143.5 mL sterile DI water were added and the mixture was well shaken. 27.85 mL tetraethylorthosilicate (TEOS) were added to the bottle. The bottle was tightly closed and well shaken.

After 1 hour waiting time the clear colorless mixture got room temperature (22° C.). Then 3.95 mL ammonia water (25%) was added quickly. The bottle was shaken by hand very well for 10 seconds and stored at room temperature. After 24 hours at room temperature additional 1.5 mL (Diethylphosphatoethyl)triethoxysilane (CAS No. 757-44-8, Gelest Corp. product code SID3412.0) were added to the mixture at room temperature. 10 mL ammonia water (25%) was added too and the reaction mixture was placed in a water bath at 85° C. for 24 hours.

At this temperature two reactions took place in parallel: The ethoxy groups of the silane got hydrolyzed, due to the high pH value. The silanol groups precipitated onto the silica nanoparticles building a surface coating on top of the nanoparticles. In parallel, but much slower, the phosphoric acid ester got hydrolyzed to yield into the corresponding acid with ammonia as the counter ion. 50 μL of the reaction mixture were placed in a one-way PMMA cuvette (10 mm width) and diluted with 1.5 mL DI water (0.2 μm filtered). The cuvette was placed in a ZetaSizer Nano for particle size measurement.

Results:
Z-average (volume based): 21.3 nm
PDI: 0.144
Intensity Peak: 27.7 nm

The nanoparticle suspension was transferred into a 500 mL round bottom flask and the ethanol as well as the ammonia gas was removed by a rotary evaporator with heated water bath. 200 mL sterile DI water was added in portions. The volume was reduced down to 120 mL. This volume was placed into 6 falcon tubes with a 100 kDa membrane (Pall Corp. Macrosep® Advance, product code MAP100038). The nanoparticle suspension was centrifuged for 10 minutes at 4,000 rpm (═2,737 g at the used centrifuge) to a volume at least 80 less than the starting volume. After centrifugation the volume in the falcon tubes was restored with sterile DI water and the centrifugation was repeated 5 times. The centrifugation step is necessary to remove excess possible unbound reaction products. After centrifugation the solid content of the collected supernatants was determined in triplicate. The method was the same as in Example 1.

Result:
Yield: 83.3 g
Solid content: 104.0 mg/mL

The nanoparticle suspension was diluted with 86.6 mL sterile DI water to get a final concentration of 50.0 mg/mL.

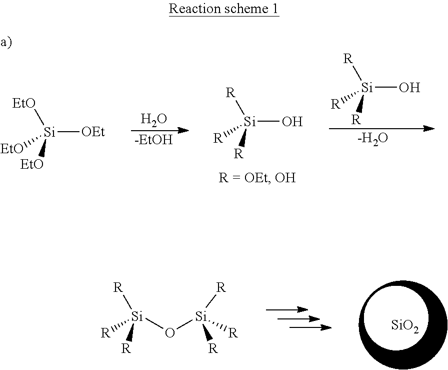

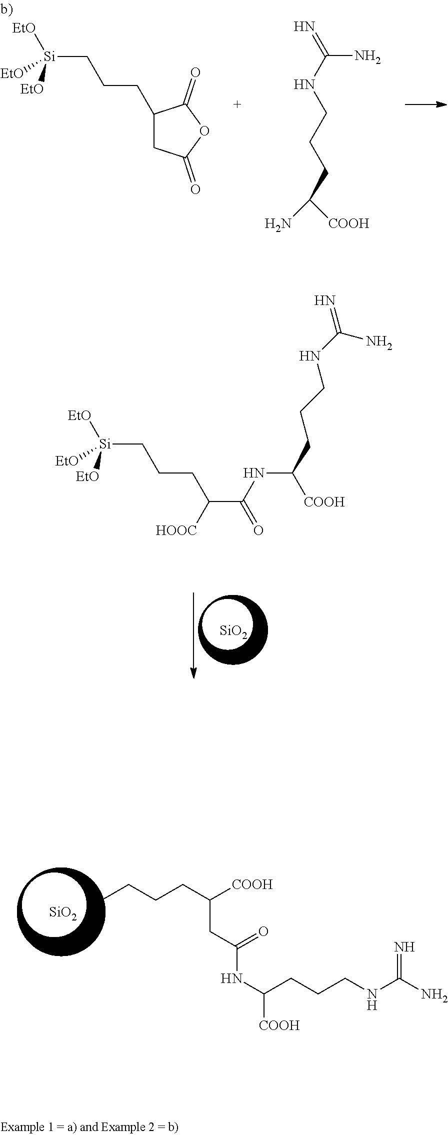

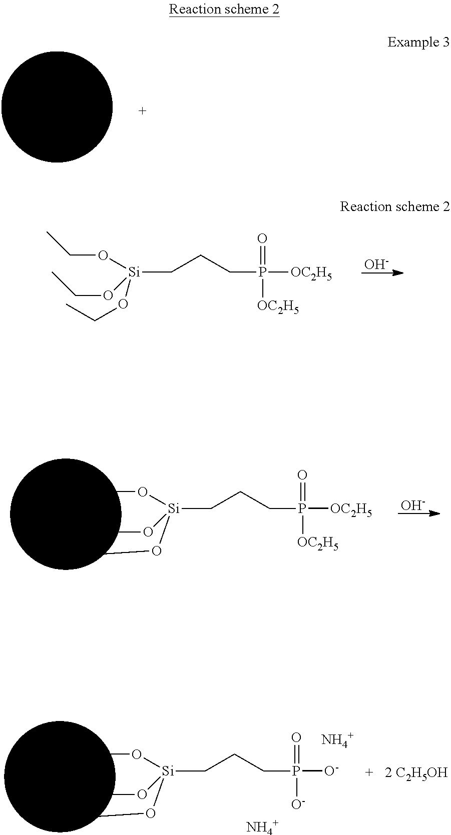

### Example 4

Preparation of Poly(I:C)@ SiO₂-Arg by Adsorptive Binding of Poly(I:C)

A mixture of 200 mL ethanol, 143.5 mL sterile de-ionized water and 27.85 mL tetraethyl orthosilicate (TEOS) was prepared. 3.70 mL ammonia, 25% (NH₃ in water) were added at room temperature. The reaction mixture was mixed vigorously by shaking for 10 seconds and left at room temperature for 24 hours without stirring. The next day another portion of 27.85 mL tetraethyl orthosilicate (TEOS) was added to the mixture. From the resulting mixture 50 μL were removed and measured by means of dynamic light scattering (DLS) on a ZetaSizer Nano ZS (Malvern). The following results were obtained:
Z-average: 25.53 nm
mean: 20.36 nm
polydispersity index (PDI): 0.136

A portion of 100 ml of the silicon dioxide particles produced before were subsequently concentrated to a volume of about 30 ml on a rotary evaporator and filled up again to 100 ml. This procedure was repeated three times to remove the ethanol and ammonia from the reaction solution. The resulting suspension was washed five times over a 100 kDa membrane with sterile deionized water. After the last washing step, the solids content of the suspension was determined gravimetrically with 7.9% SiO₂ and the suspension was adjusted to a solids content of 5.0% by adding the calculated amount of water.

500 mg L-arginine (CAS number 74-79-3; company abcr GmbH, Germany) (═2.87 mmol) was added to 15 ml of the silicon dioxide particles produced in Example 1 (750 mg SiO₂) and stirred while the arginine was completely dissolved. A clear particle suspension was obtained and to this suspension 127 μL (3-triethoxysilylpropyl) succinic anhydride (CAS number: 93642-68-3) (═0.45 mmol) were slowly added while stirring at room temperature.

2.00 ml of the “argininylated” silica nanoparticles obtained above were mixed with 3.00 ml of a low molecular weight solution of poly(I:C) (poly(I:C)-LMW from InvivoGen, Toulouse, France with a size of 0.2 to 1 kb, CAS number 31852-29-6) with a concentration of 0.833 mg poly(I:C)/mL. After thoroughly mixing with a vortex mixer for 15 seconds and after one hour, the clear suspension was mixed with 250 mg of glucose. The formulation comprises:

100 mg “argininylated” silica nanoparticles, SiO₂-Arg (c=20 mg/mL)

2.5 mg poly(I:C)-LMW (c=0.5 mg/mL)

250 mg glucose (c=50 mg/mL) (for isotonization of the formulation)

The mixture was then filled into three sterile 2.0 mL HDPE vials using a 0.2 μm sterile filter. The obtained suspension is clear and completely transparent.

The poly(I:C) loaded particles pass easily a sterile filter. Two types of filters were used: Pall Life Sciences, Acrodisc Supor® Membrane (low protein binding) 0.2 μm, cat. no. PN4602 and VWR 0.2 μm Cellulose Acetate Membrane 0.2 μm, cat. no. 514-0061.

Even after adding larger amounts of poly(I:C), the suspension remains clear and completely transparent.

### Example 5

Peptide Loading Capacity of SiO₂-Arg Nanoparticles

In a peptide loading experiment 100 μL silica nanoparticles with a diameter of 25 nm (Z-average), a solid content of 20 mg/mL and an arginylated surface were loaded with different amounts of the model peptide KKKW-Cit-SIINFEKL. To simulate physiological conditions sodium chloride (NaCl) was added to get an isotonic suspension (0.9% NaCl). Up to 10% of peptide was added to the nanoparticles. KKKW-Cit-SIINFEKL has an iso-electric point of pH 10.24, indicating cationic properties determined by 4 basic amino acids (Lysine) and 1 acidic amino acid (Glutamic acid).

50 μL of the corresponding particle-peptide-NaCl mixture was diluted with 1.5 mL sterile filtered de-ionized water and measured via dynamic light scattering (DLS) in a ZetaSizer Nano (Malvern Instruments, UK). For every sample #1 to 10 100 μL SiO₂-Arg stock solution was used. The obtained results are listed in Table 2.

TABLE 2
Results of the dynamic light scattering

| | [mg/mL] | [mg] | [%] | [μL] | [mg] | [μL] | z average | |
|---|---|---|---|---|---|---|---|---|
| # | SiO₂-Arg | peptide | peptide | Peptide stock² | NaCl | NaCl stock³ | [nm] | PDI |
| 0 | 20 | 0.00 | 0.00 | 0.0 | 0.90 | 9.00 | 23.62 | 0.07 |
| 1 | 20 | 0.02 | 1.00 | 5.1 | 0.95 | 9.45 | 24.00 | 0.10 |
| 2 | 20 | 0.04 | 2.00 | 10.2 | 0.99 | 9.92 | 24.48 | 0.10 |
| 3 | 20 | 0.06 | 3.00 | 15.5 | 1.04 | 10.39 | 26.03 | 0.14 |
| 4 | 20 | 0.08 | 4.00 | 20.8 | 1.09 | 10.87 | 26.91 | 0.14 |
| 5 | 20 | 0.11 | 5.00 | 26.3 | 1.14 | 11.37 | 28.,70 | 0.18 |
| 6 | 20 | 0.13 | 6.00 | 31.9 | 1.19 | 11.87 | 59.09 | 0.27 |
| 7 | 20 | 0.15 | 7.00 | 37.6 | 1.24 | 12.39 | 44.58 | 0.20 |
| 8 | 20 | 0.17 | 8.00 | 43.5 | 1.29 | 12.91 | 440.5 | 0.58 |
| 9 | 20 | 0.20 | 9.00 | 49.5 | 1.35 | 13.45 | 1074 | 0.31 |
| 10 | 20 | 0.22 | 10.00 | 55.6 | 1.40 | 14.00 | 2753 | 0.21 |

¹Stock solution SiO₂-Arg: 20 mg/mL SiO₂-Arg
²Stock solution Peptide: 4 mg/mL Peptide (KKKW-Cit-SIINFEKL)
³Stock solution NaCl: 100 mg/mL NaCl

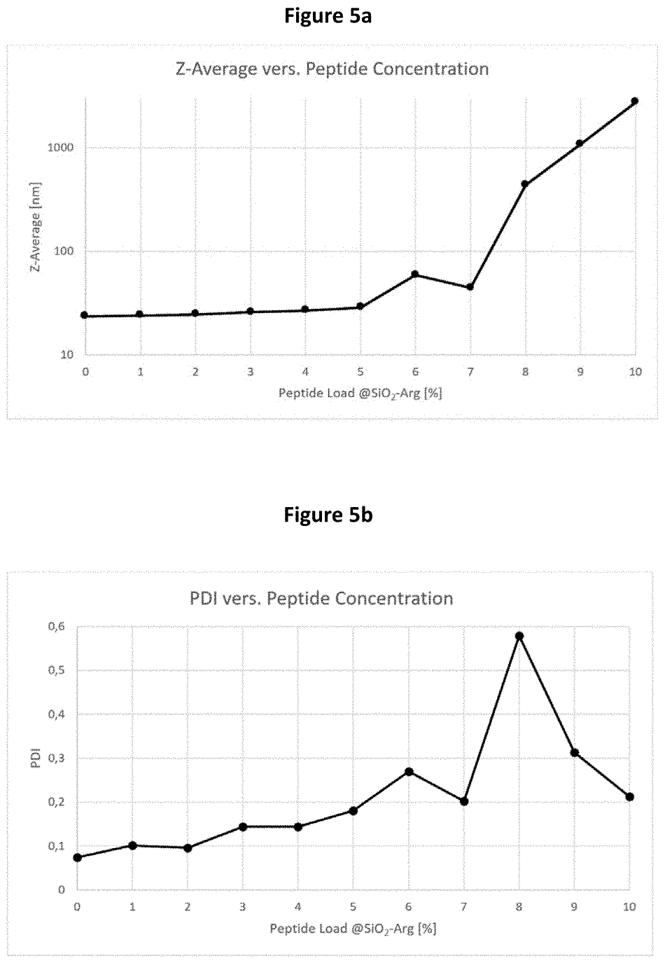

FIG. 5a illustrates the Z average versus peptide concentration and FIG. 5b the PDI versus peptide concentration.

As shown in FIGS. 5a and 5b, up to 5% by weight of this peptide can be added to the nanoparticles, to keep a stable suspension without agglomeration or precipitation. Compared to bare particles the diameter is increasing slightly (+5 nm) and the PDI indicates a still quite narrow particle size distribution. This optical clear suspension is still passing a sterile filter.

The peptide loaded particles pass easily a sterile filter. Two types of filters were used: Pall Life Sciences, Acrodisc Supor® Membrane (low protein binding) 0.2 μm, cat. no. PN4602 and VWR 0.2 μm Cellulose Acetate Membrane 0.2 μm, cat. no. 514-0061.

With increased peptide loading the particle size increases, indicating the adsorptive peptide binding to the silica nanoparticles surface. At higher peptide concentrations—in the above example 6%—the nano-suspension is collapsing, indicated by clouding and a measureable increased particle size due to agglomeration. These suspensions do not pass a sterile filter and are very unfavorable for parenteral administration.

### Example 6

Poly(I:C) Loading Capacity of SiO₂-Arg Nanoparticles

In a loading experiment 50 μL silica nanoparticles with a diameter of 25 nm (Z average), a solid content of 20 mg/mL and an arginylated surface were loaded with different amounts of poly(I:C) solution in RNase/DNase-free water by Gibco™. The poly(I:C) (LMW) was obtained from Invivogen Europe (cat. no. tlrl-picw). To simulate physiological conditions sodium chloride (NaCl) was added to get an isotonic suspension (0.9% NaCl). Up to 8% of poly(I:C) were added to the nanoparticles. The colloid remained stable: no precipitation or clouding was observable. The particle sizes and distributions, as well as the zeta potentials confirm the visual observations.

For the Zeta potential was measured according to the Smoluchowski calculation model under the following conditions:
Measurement Temp.: 25° C.
Equilibration Time: 30 s

10 to 100 runs per measurement, 5 measurements per sample (Zeta potential=mean of 5 measurements)

TABLE 3
Results of the dynamic light scattering

| [mg] | [μL] | Vol | [mg] | [μL] | Z ave | | Peak | int. Peak | Zeta |
|---|---|---|---|---|---|---|---|---|---|
| poly(I:C) | poly(I:C) stock | [μL] | NaCl | NaCl stock | [nm] | PDI | [nm] | [nm] | potential |
| — | 50 | — | 0.45 | 4.50 | 33.83 | 0.557 | 12.69 | *) | — |
| 0.00 (0%) | — | 50.0 | 0.45 | 4.50 | 22.79 | 0.077 | 20.23 | 24.47 | −27.4 |
| 0.01 (1%) | 4.0 | 54.0 | 0.49 | 4.86 | 22.92 | 0.067 | 21.42 | 24.25 | −28.1 |
| 0.02 (2%) | 8.2 | 58.2 | 0.52 | 5.23 | 23.12 | 0.075 | 19.96 | 23.97 | −28.7 |
| 0.04 (4%) | 16.7 | 66.7 | 0.60 | 6.00 | 23.35 | 0.075 | 20.04 | 25.23 | −34.2 |
| 0.06 (6%) | 25.5 | 75.5 | 0.68 | 6.80 | 23.45 | 0.08 | 19.96 | 34.93 | −36.2 |
| 0.09 (8%) | 34.8 | 84.8 | 0.76 | 7.63 | 23.97 | 0.134 | **) | 32.15 | −37.2 |

*) two peaks: 27.38 nm (67.8%) and 362.6 nm (32.2%)
**) two peaks: 7.691 nm (22.9%) and 18.4 nm (77.1%)

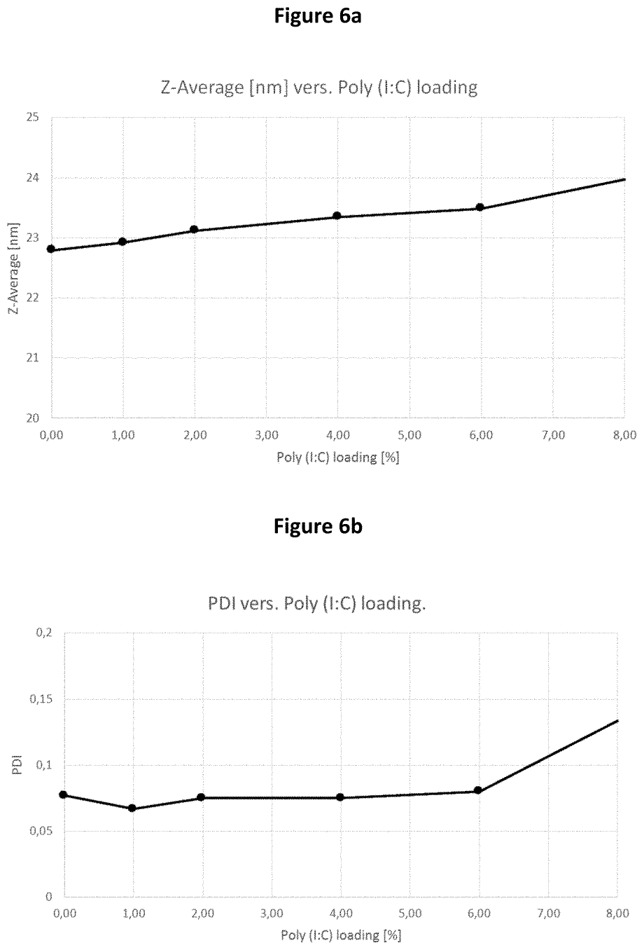

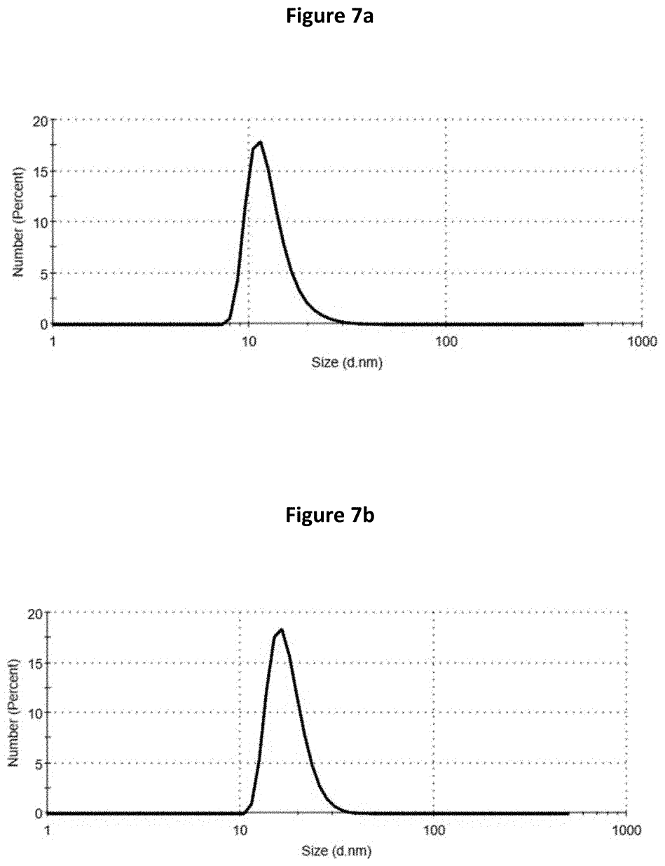

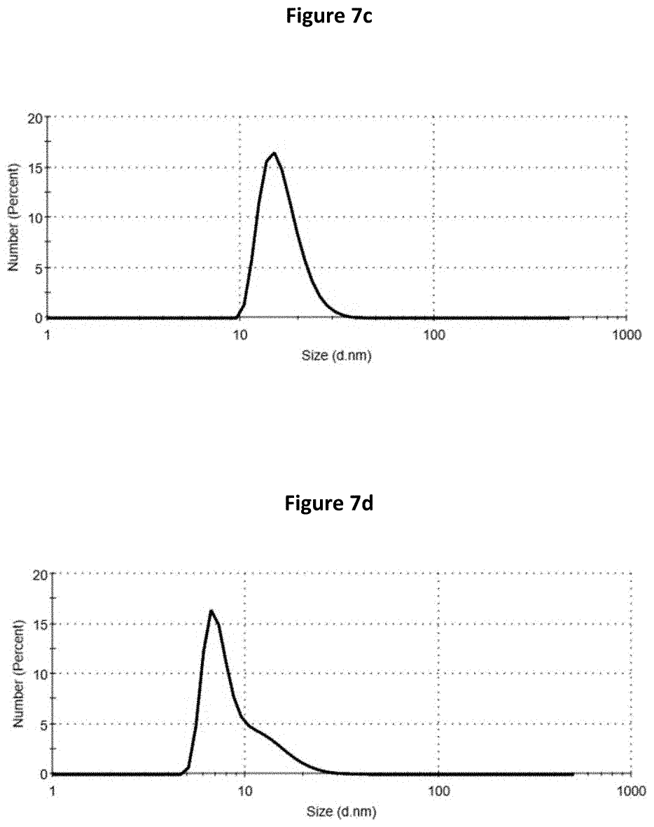

Up to a loading of 6% poly(I:C) LMW the measured Z average values increases due to increase of particle diameter by adsorbed poly(I:C) (FIG. 6b). Also the low PDI (<0.1) indicates a smooth particle loading (FIG. 6a). At 8% loading two peaks (at 7.691 and 18.4 nm) appear, also a strong peak broadening of the peak in direction to lower diameter values happens and the PDI value is getting worse (FIG. 7d). These data indicate particle “overloading”: unbound poly(I:C) generates an additional peak, resp. broadens the measurement peak.

FIGS. 7a to 7d show the Number % in a display of a ZetaSizer. The Number % is the representation of the particle distribution according to its percentage frequency.

FIG. 7a: Poly(I:C) LMW without nanoparticles

FIG. 7b: SiO₂-Arg nanoparticles without poly(I:C)

FIG. 7c: SiO₂-Arg nanoparticles loaded with 6% poly(I:C) by weight

FIG. 7d: SiO₂-Arg nanoparticles loaded with 8% poly(I:C) by weight

### Example 7

Loading of SiO₂-Arg Nanoparticles with Peptides and Poly(I:C)

In a peptide and poly(I:C) loading experiment 100 μL silica nanoparticles with a diameter of 25 nm (Z average), a solid content of 20 mg/mL and an arginylated surface were loaded with different amounts of the model peptide KKKW-Cit-SIINFEKL and with 50 μg poly IC (LMW) each (20 μL of a poly (I:C) stock solution, having a concentration of 2.5 mg/mL).

To simulate physiological conditions sodium chloride (NaCl) was added to get an isotonic suspension (0.9% NaCl). Up to 4% of peptide were added to the nanoparticles. The colloid remained stable: no precipitation or clouding was observable. The particle sizes and distributions, as well as the zeta potentials confirm the visual observations.

TABLE 5
Results of the dynamic light scattering

| [mg] | [μL] | Vol | [mg] | [μL] | [μg] | z ave | | int. Peak | Peak | Zeta |
|---|---|---|---|---|---|---|---|---|---|---|
| pept.* | pept.* stock | [μL] | NaCl | NaCl stock | Poly(I:C) | [nm] | PDI | [nm] | [nm] | potential |
| 0.04 (2%) | 10.2 | 110.2 | 0.99 | 9.92 | 50 | 24.09 | 0.172 | 24.57 | 19.48 | −37.1 |
| 0.06 (3%) | 15.5 | 115.5 | 1.04 | 10.39 | 50 | 27.87 | 0.279 | 25.7 | 21.31 | −27.6 |
| 0.08 (4%) | 20.8 | 120.8 | 1.09 | 10.87 | 50 | 28.47 | 0.305 | 25.41 | 20.76 | −25.9 |

*(pept. = peptide)

### Example 8

Loading of SiO₂-Arg Nanoparticles with ssRNA (Poly(U))

In an ssRNA loading experiment 400 μL silica nanoparticles with a diameter of 25 nm (Z average), a solid content of 50 mg/mL and an arginylated surface (in total 20 mg silicon dioxide) were loaded with 500 μL poly(U) solution with a concentration of 1.0 mg/mL (in total 0.5 mg poly(U) and 100 μL sodium chloride (NaCl) with a concentration of 90 mg/mL was added to get an isotonic suspension (0.9% NaCl). The loading of poly(U) on silica nanoparticles in this case is 2.44% by weight. 50 μL of this sample were measure by DLS. The poly(U) was obtained from InvivoGen Europe, Toulouse, France (Cat. Code: tlrl-sspu).

TABLE 5
Results of the dynamic light scattering

| [mg] | [mg] | [mg] | [μL] | Z ave | | Peak | int. Peak | Zeta |
|---|---|---|---|---|---|---|---|---|
| poly(U) | SiO2-Arg | NaCl | total volume | [nm] | PDI | [nm] | [nm] | potential |
| 0.5 (2%) | 20.0 | 90 | 1000 | 23.19 | 0.065 | 20.21 | 24.93 | −31.6 |

### Example 9

Loading of SiO₂-Arg Nanoparticles with Unmethylated DNA (CpG ODN)

In a DNA loading experiment 40 μL silica nanoparticles with a diameter of 25 nm (Z average), a solid content of 50 mg/mL and an arginylated surface (in total 2 mg silicon dioxide) were loaded with 50 μL ODN 2395 solution with a concentration of 1.0 mg/mL (in total 0.5 mg ODN 2395 and 10 μL sodium chloride (NaCl) with a concentration of 90 mg/mL was added to get an isotonic suspension (0.9% NaCl). The loading of on silica nanoparticles in this case is 2.44% by weight. 50 μL of this sample were measure by DLS.

ODN 2395 was obtained from InvivoGen, France (cat. code: tlrl-2395). It is a 22mer with the structure: 5′-tcgtcgttttcggccc:gcgcc-3′ (bases are phosphorothioate (nuclease resistant), palindrome is underlined)

TABLE 6
Results of the dynamic light scattering

| [mg] | [mg] | [mg] | [μL] | Z ave | | Peak | int. Peak | Zeta |
|---|---|---|---|---|---|---|---|---|
| CPG ODN | SiO2-Arg | NaCl | total volume | [nm] | PDI | [nm] | [nm] | potential |
| 0.05 (2.44%) | 2.0 | 9 | 100 | 23.88 | 0.083 | 20.76 | 25.19 | −28.5 |

### Example 10

Example for Solubility Enhancement of Epitopes

On the N-terminal site of the enzymatically cleaved linker the addition of polar amino acids can be used to enhance the solubility of the whole peptide. The HLA-A:02 immunogenic HPV 16 E782-90 epitope LLMGTLGIV (SEQ ID NO: 233), for example, is extremely non-polar and has a very bad solubility in water. The use in a human vaccine is difficult. In animal studies researchers dissolve it in DMSO. The solubility after e.g. subcutaneous injection is questionable: dilution might result in precipitation of large peptide particles, which is very unfavorable for transport to lymphnodes.

According to the invention the epitope LLMGTLGIV was enlarged on the N-terminal site with the enzymatic cleavable linker Val-Cit and the cationic solubilizing sequence Lys-Lys-Lys, leading to
KKKV-Cit-LLMGTLGIV.

The complete synthesis was done by automated microwave supported solid phase peptide synthesis (SPPS) in one run.

This peptide has an excellent solubility in water. After endosomal and/or cytosolic cleavage by cathepsin B the native HPV 16 E782-90 is released. Also the remaining KKKV-Cit is not immunogenic, due to the fact it's too short to be presented at any MHC class I or MHC class II.

### Example 11

Human TLR-induced Cytokines in the Primary Human Macrophage Model (THP-1)

Production of cytokines after stimulation of differentiated THP-1 cells with poly(I:C), SiO₂-Arg and poly(I:C)@ SiO₂-Arg were performed with Multi-Analyte ELISArray from Qiagen. Nanoparticle diameter is 22.1 nm (Z-average), PDI 0.08.

All experiments were performed with low molecular weight poly(I:C)-LMW from InvivoGen Europe, Toulouse, France.

The screening of cytokines showed significant production of cytokines:

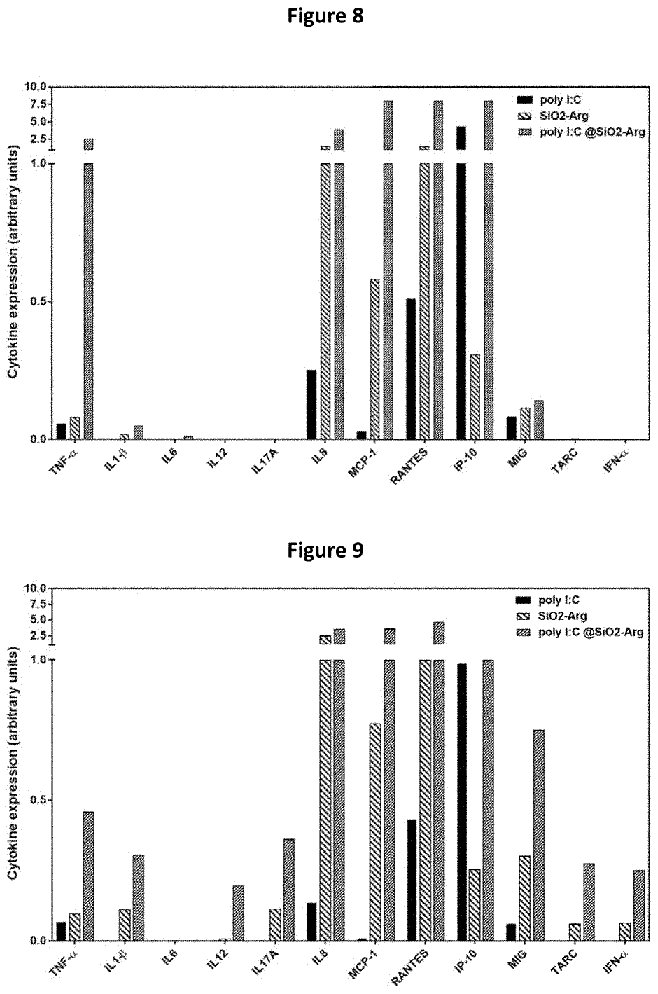

FIG. 8: Cytokine expression (arbitrary units) for different types of cytokines after 72 h stimulation

TNF-α, IL8 (CXCL8), MCP-1 (CCL2), RANTES (CCL5), IP-10 (CXCL10), MIG (CXCL9) were highly expressed after 72 h stimulation with poly(I:C) @SiO₂-Arg. High cytokine release of IL8 (CXCL8), MCP-1 (CCL2), RANTES (CCL5), IP-10 (CXCL10), MIG (CXCL9) was also noticed for SiO₂-Arg nanoparticles.

FIG. 9: Cytokine expression (arbitrary units) for different types of cytokines after 96 h stimulation

After 96 h stimulation with poly(I:C) @SiO₂-Arg, the expression rate of cytokines IL1-P, IL12, IL17A, TARC, IFN-α is increased. High cytokine release of IL8 (CXCL8), MCP-1 (CCL2), RANTES (CCL5), IP-10 (CXCL10), MIG (CXCL9) was also noticed for SiO₂-Arg nanoparticles.

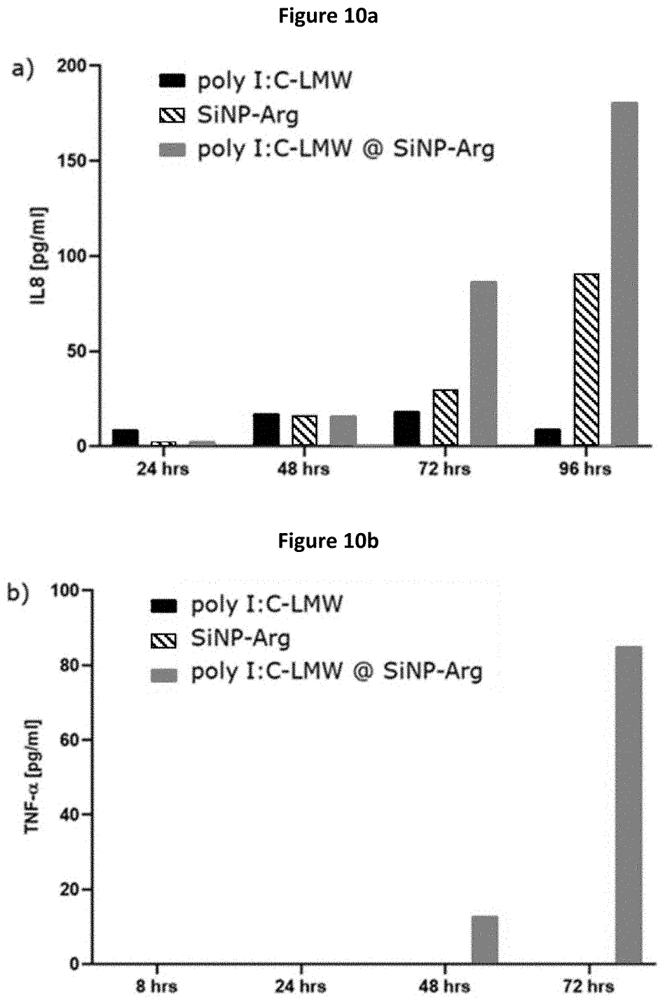

To investigate whether the combination of the TLR3 agonist poly(I:C)-LMW and SiO₂-Arg is able to stimulate cytokine release, the differentiated human macrophage-like THP-1 cells were incubated with poly(I:C)-LMW adsorptively bound to SiO₂-Arg (poly(I:C)@ SiO₂-Arg) or with the individual compounds and then subjected to cytokine-specific enzyme-linked immunosorbent assay (ELISA). The supernatant of the THP-1 cells was analyzed for interleukin 8 (IL-8) and tumor necrosis factor α (TNF-α) at several time points (FIG. 10). IL-8 and TNF-α are important mediators of the innate immune system response, regulating the activity of various immune cells. The release of these two cytokines is proof of a successful stimulation of the immune system.

FIGS. 10a and 10b: Cytokine release at different time points after stimulation of differentiated THP-1 cells with poly(I:C) [12.5 μg/ml], SiO₂-Arg [0.5 mg/ml] or the novel adjuvant (poly(I:C) [12.5 μg/ml] bounded on SiO₂-Arg [0.5 mg/ml]).

FIG. 10a: Quantification of IL-8 release was performed using ELISA MAXTMDeluxe Set Human IL-8 from BioLegend, USA.

FIG. 10b: Quantification of TNF-α release was performed using ELISA MAXTMDeluxe Set Human TNF-α from BioLegend, USA.

### Example 12

Experiments in the Influenza A Model of the Mouse

For the determination of the immunostimulatory potency of TLR3 agonists, a suspension is prepared according to Example 6 was tested in an in vivo study together with other active compounds as immunostimulators with regard to its prophylactic effect against a five-fold LD50 dose of the influenza A virus PR8/34.

The following active compounds were tested:
Placebo: glucose solution (5%)
free poly(I:C) LMW: Low Molecular Weight poly(I:C) comprises short strands of inosinic acid poly(I) homopolymer annealed to strands of cytidinic acid poly(C) homopolymer. The average size of poly(I:C) LMW is from 0.2 kb to 1 kb (InvivoGen, France) in glucose solution (5%)
ZelNate®: FDA approved immunostimulant that aids in the prophylaxis of Bovine Respiratory Disease (BRD) due to Mannheimia haemolytica (Bayer AG, Germany)
Poly(I:C)@SiO₂-arginylated prepared according to Example 6
Silicon nanoparticles unmodified with a diameter of 25 nm in 5% glucose solution

TABLE 7
Overview of prophylaxis trials in mice, every group consists of ten mice

| Group | | | |
|---|---|---|---|
| No. | active compound | composition | H1N1-dose; admin. |
| A | Placebo | — | 50 TCID₅₀ (5 × LD₅₀); i.n. |
| B | Zelnate ® | 100 μL | 50 TCID₅₀ (5 × LD₅₀); i.n. |
| C | poly(I:C) (LMW) | 50 μg in 100 μL | 50 TCID₅₀ (5 × LD₅₀); i.n. |
| D | SiO₂ | 2 mg SiO₂; | 50 TCID₅₀ (5 × LD₅₀); i.n. |
| | (25 nm) | (unmodified) | |
| E | poly(I:C)@SiO₂- | 2 mg SiO₂- | 50 TCID₅₀ (5x LD50); i.n. |
| | Arg (25 nm) | “argynilated” | |
| | | and | |
| | | 50 μg poly(I:C) | |
| | | (LMW) in | |
| | | 100 μL | |

In all cases the injection volume was 100 μL.

Each animal group (A to E), consisting of ten C57BL/6 mice, was treated 24 hours before administration of the influenza A virus subcutaneously with the respective active compound or placebo. The virus was administered intranasally.

In this study the body weight was used as a reliable and easy-to-measure marker for the animal health. Sick animals eat less and lose weight very quickly. For ethical reasons, the study defined a body weight loss of 25%, based on the body weight on the day of the virus administration, as the termination criterion. In contrast, in many publications from older studies, the “termination criterion” is the death of the animals due to the viral disease.

In this study, the formulation of poly(I:C) prepared according to Example 6, which is adsorptively bound to silica nanoparticles with an “argininylated” surface, showed a surprisingly strong immunostimulatory effect, which resulted in a statistically significantly longer survival rate of the animals of group E and to a significantly less weight loss.

The free poly(I:C), which is not bound to nanoparticles (group C), with the same concentration of active ingredient, does not show any statistically significant improvement compared to the placebo group (group A). The unmodified silica nanoparticles (group D) also showed no statistically significant improvement.

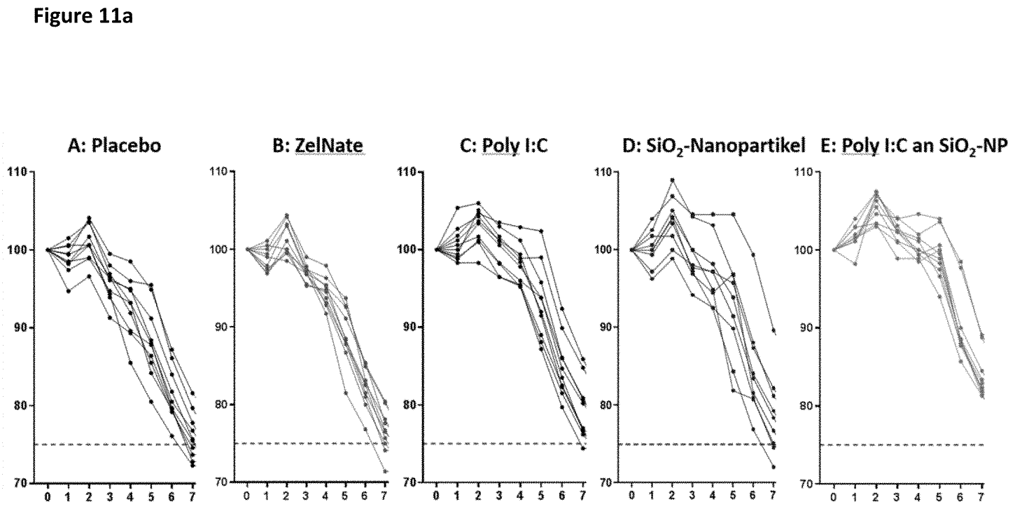

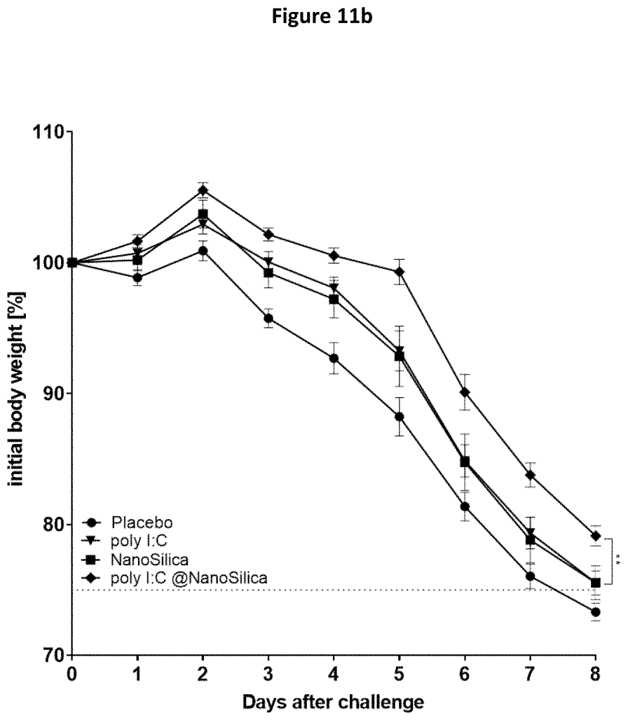

From the comparative experiments above, it could be shown that a TLR3 agonist which is adsorptively bound to silica nanoparticles (group E), is clearly superior over the free TLR3 agonist (group C) which was applied in the same concentration and under identical test conditions. The immune booster ZelNate® (group B) also showed no significant improvement in this study compared to the placebo group (group A). This also applies to the unmodified silica nanoparticles of group D, where no significant effect could be observed (FIGS. 11a and 11b).

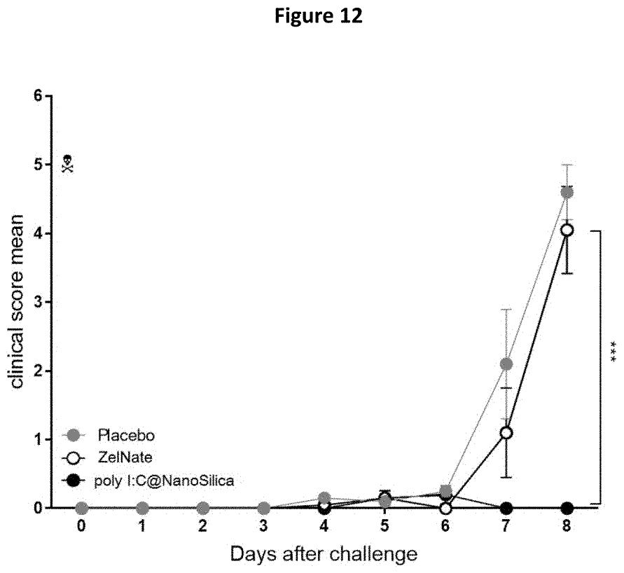

FIG. 12 shows the clinical scores. Data are presented as mean clinical score (maximum score=5; death of animals, abort of experiment)±SEM (n=10) in relation to days after challenge.

### Example 13

Generation of HPV16 E7₁₁₋₁₉ Specific CD8 T Cell Immune Responses in Tumor-Free A2.DR1 Mice

The immunogenicity of human HPV16 E6/E7-derived, HLA-A2-binding epitopes can only be studied in genetically modified mice. Therefore the in vivo studies were performed using the HLA-humanized A2.DR1 BL6 mice. A2.DR1 mice are a highly sophisticated mouse model since they underwent a multitude of genetic alterations to exhibit the HLA-A2+/HLA-DR1+, H-2-phenotype and shown to assemble functional CD4⁺ and CD8⁺ T cell responses against multiple epitopes restricted by HLA-A2 and HLA-DR1. (Pajot, A. et al., A mouse model of human adaptive immune functions: HLA-A2.1-/HLA-DR1-transgenic H-2 class I-/class II-knockout mice, European Journal of Immunology, 2004, Vol. 34, p. 3060-3069).

The efficacy of various formulations was examined for their ability to induce high frequencies of immunogenic fragment-specific CD8⁺ T cells after three weekly, subcutaneous immunizations (Day 0: Prime, Day 7: Boost, Day 14: Boost, Day 21: Spleen Sampling).

Used Substances:
poly(I:C)-HMW: The average size of poly(I:C)-HMW is 1.5 to 8 kb, InvivoGen, France
SiO₂—PO₃H₂ according to Example 3

Preparation of KKKW-Cit-E7₁₁₋₁₉+ poly(I:C)-HMW @ SiO₂-Arg:

2.72 mg (1.48 μmol) KKKW-Cit-E7₁₁₋₁₉ are dissolved in 592 μL DNase/RNase free distilled water under sterile conditions. The solution is added under vortexing to 2.37 ml of a 50 mg/mL stock solution of SiO₂-Arg. 296 mg solid glucose are added and the solution is mixed until the solid has completely dissolved. Finally, 2.96 mL poly(I:C)-HMW is added as a 1.0 mg/mL stock solution while the sample is vortexed. The vaccine is filtered through a 0.45 μm filter. 1.8 mL each are filled into 3 separate vials (1 vial per immunization) and stored at 4° C.

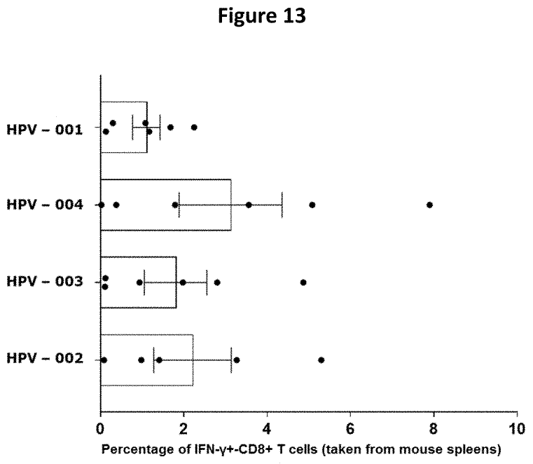

The influence of the N-terminal peptide elongation of the immunogenic fragment HPV16 E7₁₁₋₁₉ (SEQ ID NO: 174=YMLDLQPET) as well as the influence of the nanoparticles surface modification were investigated. To evaluate the vaccine-induced HPV16 E7₁₁₋₁₉-specific CD8⁺ T cells, the ex vivo restimulated splenocytes were assessed in an IFN-γ intracellular cytokine staining (ICS) followed by flow cytometry (FIG. 13).

FIG. 13:

Y-Axis:
HPV-001: RW-Cit-E7₁₁₋₁₉ [50 nmol]+poly(I:C)-HMW [50 μg]
HPV-002: RW-Cit-E7₁₁₋₁₉ [50 nmol]+poly(I:C)-HMW [50 μg] @ SiO₂-Arg [2.25 mg]
HPV-003: RW-Cit-E7₁₁₋₁₉ [50 nmol]+poly(I:C)-HMW [50 μg] @ SiO₂—PO₃H₂ [2.25 mg]
HPV-004: KKKW-Cit-E7₁₁₋₁₉ [50 nmol]+poly(I:C)-HMW [50 μg] @ SiO₂-Arg [2.25 mg]

X-Axis:

Percentage of IFN-γ+-CD8+ T Cells (Taken from Mouse Spleens)

Frequency of IFN-γ positive E7₁₁₋₁₉ specific CD8⁺ T cells after ex vivo stimulation of splenocytes with E7₁₁₋₁₉ in the presence of Golgi apparatus-transport-inhibitors. After subsequent IFN-γ ICS, IFN-γ positive E7₁₁₋₁₉ specific T cells were determined by flow cytometry. Data are represented as the mean+/−SEM. Each dot represents one mouse.

As shown in FIG. 13, three immunizations with nanoparticles conjugated to epitope were able to induce antigen-specific splenic IFN-γ⁺ CD8⁺ T cells at higher frequencies in comparison to free epitope injections. Furthermore, the direct comparison of two surface modifications of nanoparticles (HPV-002 vs. HPV-003) demonstrated a better performance when the epitope was conjugated to SiO₂-Arg (HPV-002).

Moreover, the influence of the N-terminal peptide elongation (HPV-002 vs. HPV-004) was analyzed, where the “KKKW-Cit-” modification (HPV-004) led to higher frequencies of epitope-specific CD8⁺ T cells.

This experiment successfully demonstrated the ability of the HPV16 vaccines according to the invention to induce a high frequency of epitope specific CD8⁺ T cells. In summary, SiO₂-Arg as well as the “KKKW-Cit-” peptide elongation have shown their beneficial properties over SiO₂—PO₃H₂ nanoparticles and the “RW-Cit-” peptide elongation. Therefore, the combination of both was used for assessing therapeutic efficacy in a tumor study.

### Example 14

Therapeutic Efficacy in the PAP-A2-HPV16 Tumor Model

The final study goal is the development of a therapeutic anti-HPV16 vaccine, which would be given to patients diagnosed with either a precursor lesion or an established cancer. Therefore, the ability of the novel vaccines was tested to induce control of tumor growth in a therapeutic vaccination experiment.

Immunization schedule for early therapeutic treatment study in the PAP-A2-HPV16 tumor model: (Kruse, S. Therapeutic vaccination against HPV-positive tumors in a MHC-humanized mouse model, Ruperto Carola University Heidelberg, 2019, Dissertation; Kruse et al., Therapeutic vaccination using minimal HPV16 epitopes in a novel MHC-humanized, Oncoimmunology, 2019, Vol. 8, 1, p. e1524694):

To evaluate the therapeutic efficacy of SiO₂-Arg conjugated with HPV16 E7-derived immunogenic fragment, the HPV16 E6⁺/E7⁺ PAP-A2 tumor model in HLA-humanized A2.DR1 BL6 mice was used. 1.5-10⁶ PAP-A2 cells were injected subcutaneously, which should result in large tumors within 2 to 3 weeks. Starting with day 4 after tumor inoculation, the tumor-bearing mice were treated weekly (3 immunizations total, Prime-Boost-Boost) with the complete vaccine or with the individual compounds as controls, until the ethical endpoint (tumor volume 1000 mm³) was reached.

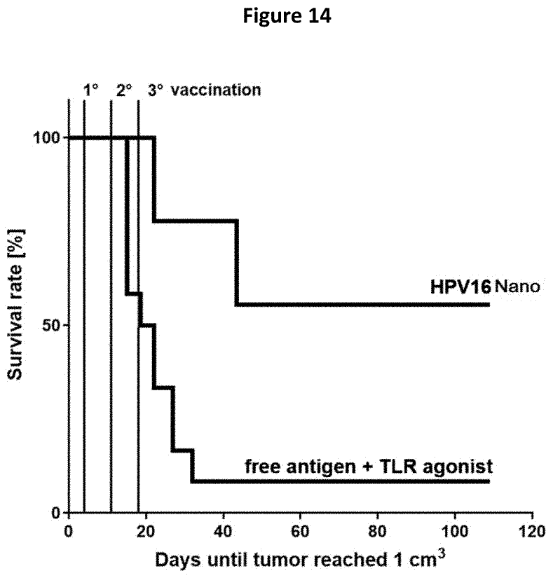

FIG. 14 shows the survival rate of mice, either receiving the free antigen HPV16 E7 YMLDLQPET (SEQ ID NO: 174)+poly(I:C) (HMW, high molecular weight), shown as “free antigen+TLR agonist” or KKKW-Cit-YMLDLQPET+poly(I:C) (HMW) both adsorptively bound to arginylated silica nanoparticles having a diameter of 23 nm, shown as “HPV16Nano”.

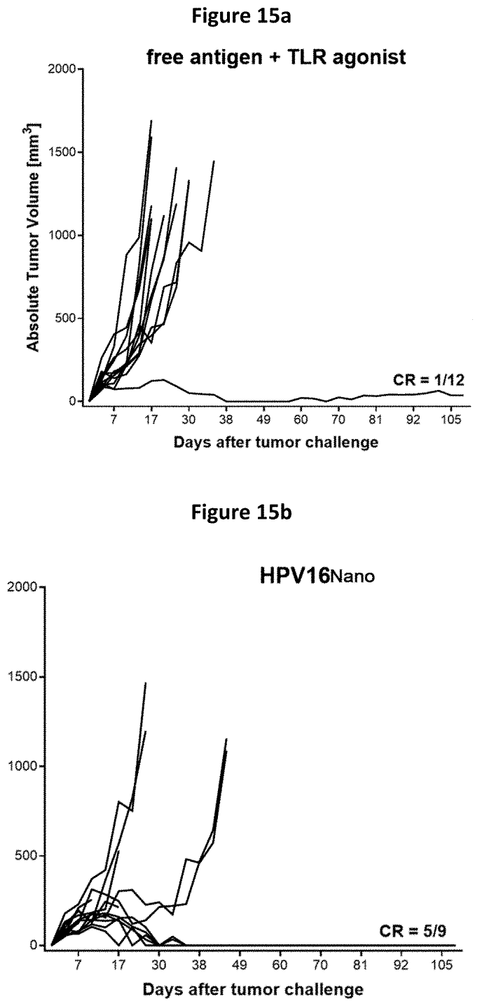

As shown in FIG. 14, the treatment of tumor-bearing mice with KKKW-Cit-YMLDLQPET+poly(I:C) (HMW) both adsorptively bound to arginylated silica nanoparticles (HPV16Nano) resulted in complete tumor regression (CR) in 5 out of 9 mice with overall survival rate of 55%. In contrast, 90% of carrier control (free antigen+TLR3 agonist) mice had to be eliminated due to excessive tumor growth. FIG. 15a and FIG. 15b show the individual tumor growth of both groups.

Taking together the results from therapeutic vaccinations with single compounds and with SiO₂-Arg conjugated with epitope, it can be concluded that the best therapeutic anti-tumor results were achieved with the triple surface-arginylated nanoparticles conjugated with KKKW-Cit-YMLDLQPET vaccination.

### Example 15

Cross-Presentation Experiments

MHC Class I Cross Presentation of OVA₂₅₇₋₂₆₄ after Intracellular Processing in DC2.4 Cells

To demonstrate the functionality of the enzymatic cleavage (W-Cit) site attached to an immunogenic epitope, experiments on cross-presentation of the model antigen OVA₂₅₇₋₂₆₄ (═SIINFEKL in one letter code for amino acids) in murine dendritic cells (DC2.4 cells, immortalized murine dendritic cells) were performed. Therefore, the expression of SIINFEKL on MHC class I after incubation with various OVA-derived constructs, with and without nanoparticles, was determined by antibody (25-D1.16, PE/Cy7 anti-mouse H-2Kb bound to SIINFEKL Antibody, Biolegend, Inc., USA) specific detection of the native epitope presented on MHC class I. Free, not MHC class I bound epitope or elongated/modified or other epitopes on MHC class I are not recognized by the antibody, which is highly selective to SIINFEKL presented on the MHC class I molecule H-2Kb.

In the experiment, the OVA₂₅₇₋₂₆₄ presentation efficacy after incubation of 5-104 DC2.4 cells with 5 μM solutions of full length protein (OVA=Ovalbumin), N- and C-terminal elongated epitope (OVA₂₄₇₋₂₆₄A₅K, a so called synthetic long peptide (SLP)), N-terminal elongated epitope with a Cathepsin B cleavable sequence (exemplary shown RW-Cit-OVA₂₅₇₋₂₆₄) or native epitope (OVA₂₅₇₋₂₆₄), each with and without nanoparticles, was compared. Surface phosphorylated silica nanoparticles (SiO₂—PO₃H₂) with an average diameter of 25 nm (comparable size to SiO₂-Arg used in other experiments) were used as carrier. After an incubation time of 6 h, the test substances were removed by washing with phosphate-buffered saline (PBS). In the next step, the cells were incubated with CD16/CD32 antibody in order to block the non-specific binding of the detection antibody to the Fc (Fragment, crystallizable) receptor of the cells. After incubation with 25-D1.16 antibody, the amount of cross-presented OVA₂₅₇₋₂₆₄ was quantified by flow cytometry.

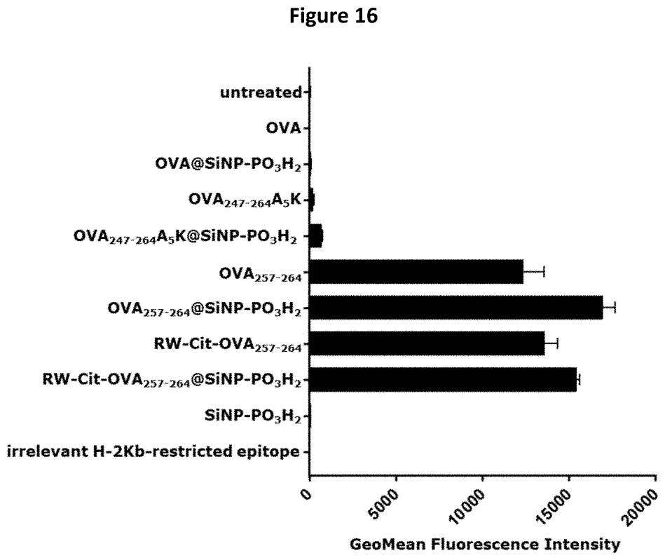

FIG. 16: OVA₂₅₇₋₂₆₄ MHC class I presentation after 6 h incubation of H₂-Kb positive cells with 5 μM solution of native or elongated OVA₂₅₇₋₂₆₄ epitope or full length OVA protein with or without SiO₂—PO₃H₂. Quantification was carried out by flow cytometry after labeling with 25-D1.16 detection antibody.

As shown in FIG. 16, only the native epitope and the elongated epitope with an enzymatic cleavage site are presented in significant amounts on the MHC. The uptake and thus the amount of presented epitope can be increased slightly by incubation with peptides adsorptively bound to nanoparticles. While the native epitope does not necessarily have to be internalized into the cell, since it can also be loaded exogenously onto the MHC molecule, the N-terminal elongated epitope must be internalized to release the native sequence. The detection of the native epitope on MHC class I after incubation of the cells with RW-Cit-OVA₂₅₇₋₂₆₄ confirms a proof of the functionality of the enzymatic cleavage site.

### Example 16

Stability Measurement in Human Serum

HEK-Blue™ hTLR3 Cells are designed to measure the stimulation of human TLR3 by monitoring the activation of NF-kB. HEK-Blue™ hTLR3Cells were obtained by co-transfection of the hTLR3 gene and an optimized secreted embryonic alkaline phosphatase (SEAP) reporter gene placed under the control of an NF-kB and AP-1-inducible promoter into HEK293 cells. Stimulation with a TLR3 ligand activates NF-kB and AP-1 which induce the production of SEAP. Levels of SEAP can be easily determined with HEK-Blue™ Detection, a cell culture medium that allows for real-time detection of SEAP. The hydrolysis of the substrate by SEAP produces a purple/blue color that can be easily detected with the naked eye or measured with a 96 well plate reader. (Invivogen, see https://www.invivogen.com/hek-blue-htlr3).

Poly(I:C) and poly(I:C)@SiO₂-Arg were exposed for 60 minutes at 37° C. to human serum (HS). The serum was used in concentrations of 5, 10 and 20%. A serum concentration of 20% corresponds quite well to the composition of peripheral lymph.

Poly(I:C) and poly(I:C)@SiO₂-Arg were added separately to human serum to get a concentration of 1 μg poly(I:C)/mL. In case of poly(I:C)@SiO₂-Arg the SiO₂ concentration was 40 μg/mL. The poly(I:C) payload at SiO₂-Arg in this case was about 2.5%. After exposure to HS 20 μL of the medium were taken and added to 180 μL HEK-Blue™hTLR3 cells in HEK-Blue™ Detection medium. This mixture was incubated for 13 hours at 37° C. and the plate was analyzed in a 96 well plate reader (Tecan Reader, Type: Infinite M200 Pro).

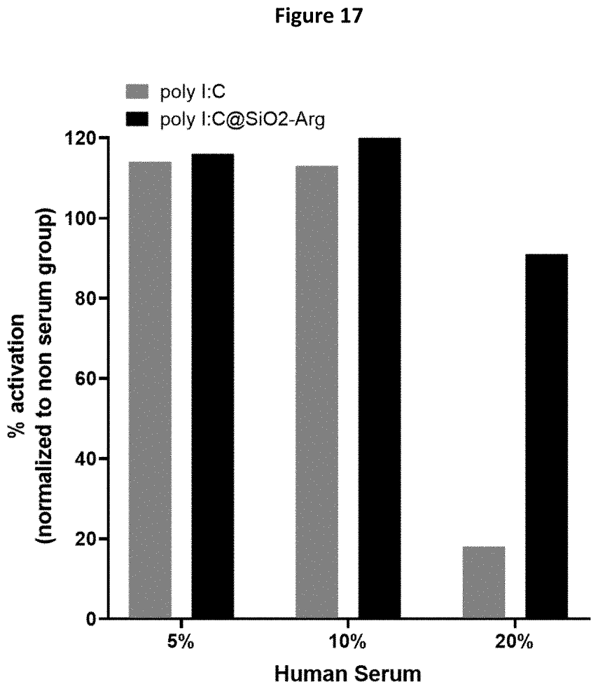

The results are shown in FIG. 17.

The calculated half-life of free poly(I:C) in 20% human serum under the chosen conditions is 24.2 minutes (18% of initial concentration after 60 minutes). Half-life calculation:

t_{1/2}=ln 2/(ln 100/ln 18)*60 [min]

The half-life for poly(I:C)@SiO₂-Arg under the same conditions is calculated to 395 minutes (90% of initial concentration after 60 minutes)

t_{1/2}=ln 2/(ln 100/ln 90)*60 [min]

So the attachment of poly(I:C) to arginylated silica nanoparticles increases the half-life by a factor of 16 (═395/24.2).

### Example 17

Stability Measurement in Bovine Serum

HEK-Blue™ hTLR3 Cells are designed to measure the stimulation of human TLR3 by human TLR3 by monitoring the activation of NF-kB. HEK-Blue™hTLR3Cells were obtained by co-transfection of the hTLR3 gene and an optimized secreted embryonic alkaline phosphatase (SEAP) reporter gene placed under the control of an NF-kB and AP-1-inducible promoter into HEK293 cells. Stimulation with a TLR3 ligand activates NF-kB and AP-1 which induce the production of SEAP. Levels of SEAP can be easily determined with HEK-Blue™ Detection, a cell culture medium that allows for real-time detection of SEAP. The hydrolysis of the substrate by SEAP produces a purple/blue color that can be easily detected with the naked eye or measured with a 96 well plate reader (Invivogen).

Poly(I:C) and poly(I:C)@SiO₂-Arg were exposed for 60 minutes at 37° C. to fetal bovine serum (FBS). The serum was used in concentrations of 5, 10 and 20%. A serum concentration of 20% corresponds quite well to the composition of peripheral lymph.

Poly(I:C) and poly(I:C)@SiO₂-Arg were added separately to bovine serum to get a concentration of 1 μg poly(I:C)/mL. In case of poly(I:C)@SiO₂-Arg the SiO₂ concentration was 40 μg/mL. The poly(I:C) payload at SiO₂-Arg in this case was about 2.5%. After exposure to FBS 20 μL of the medium were taken and added to 180 μL HEK-Blue™ hTLR3 cells in HEK-Blue™ Detection medium. This mixture was incubated for 13 hours at 37° C. and the plate was analyzed in a 96 well plate reader (Tecan Reader, Type: Infinite M200 Pro).

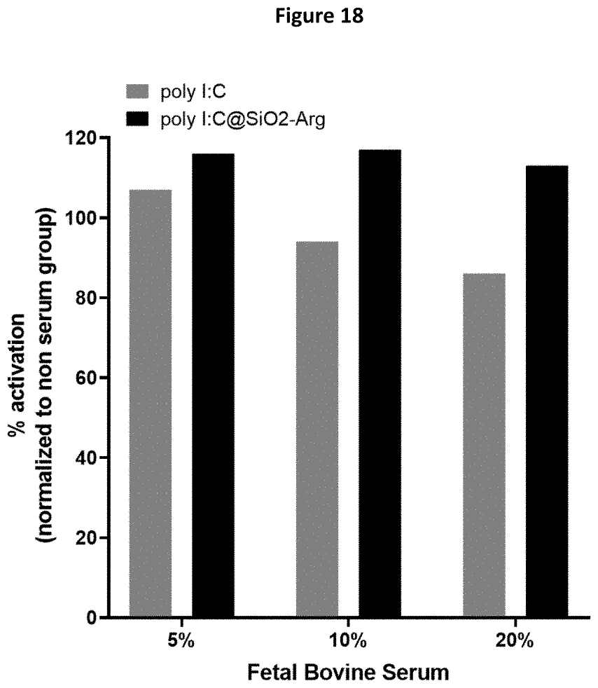

The results can be seen in FIG. 18:

While the free poly(I:C) shows clear signs of degradation at higher serum concentrations, poly(I:C)@SiO₂-Arg shows markedly higher stability. Half-life calculation for poly(I:C)@SiO₂-Arg is useless, due to a very low decay rate.

1. Composition comprising nanoparticles which are loaded with pharmaceutically acceptable compounds comprising one or more human papilloma virus (HPV)-derived immunogenic fragments or a variant thereof, having silicon dioxide and functional groups on the surface, wherein
the functional groups are capable to carry and/or stabilize both negative and positive charges of the pharmaceutically acceptable compounds,
the zeta potential of the composition has a value of at least ±15 mV,
the nanoparticles have a particle size below 150 nm.

2. Composition according to claim 1 characterized in that the immunogenic fragment is derived from the E6 and/or E7 proteins of HPV16 and/or HPV18, preferably selected from SEQ ID NOs: 5-257.

3. Composition according to claim 1 characterized in that the immunogenic fragment is at least 75% identical to an immunogenic fragment derived from the E6 and/or E7 proteins of HPV16 and/or HPV18, preferably selected from SEQ ID NOs: 258-295.

4. Composition according to claim 1 characterized in that the immunogenic fragment is able to bind to major histocompatibility complex (MHC) class I and/or MHC class II.

5. Composition according to claim 1 characterized in that the immunogenic fragment is able to induce a cytotoxic T cell response.

6. Composition according to claim 1, wherein the nanoparticles are loaded with pharmaceutically acceptable compounds comprising one or more human papilloma virus (HPV)-derived immunogenic fragments and polyinosinic:polycytidylic acid (poly(I:C)) or any derivatives thereof.

7. Composition according to claim 1, wherein the nanoparticles have a surface loading density up to 0.5, preferably between 0.01 and 0.5, more preferred between 0.03 and 0.4, most preferred between 0.05 to 0.3, in relation to the total number of the pharmaceutically acceptable compounds with regard to the surface of the nanoparticle in nm² [molecules/nm²].

8. Composition according to claim 1, wherein the net charge of the loaded nanoparticles in total is different to zero.

9. Composition according to claim 1, wherein the functional groups are connected to a linker L which is linked to the surface of the nanoparticles by way of a covalent or adsorptive bond.

10. Composition according to claim 9, wherein the linker compound L comprises at least one carboxyl (—COOH) or carboxylate (—COO—) group as functional group.

11. Composition according to claim 9, wherein the linker compound L comprises at least one guanidino-group (—NHC(═NH)NH₂ or —NHC(═NH₂+)NH₂) or amino-group (—NH₂ or —NH₃⁺) group as a functional group.

12. Composition according to claim 1, wherein the pharmaceutically acceptable compound is conjugated to the nanoparticle by adsorptive attachment.

13. Composition according to claim 1 for use as vaccines or as immunostimulant.

14. Composition according to claim 1 for use as vaccines for the prevention of HPV infection or the treatment of HPV positive humans or the treatment of HPV positive tumors.

15. Vaccine comprising compositions according to claim 1.
